(19) United States
(12) Patent Application Publication
(10) Pub. No.: US 2025/0136629 A1
(43) Pub. Date: May 1, 2025

(54) SELF-ASSEMBLED NANOPARTICLES FOR PHOTOTHERMAL THERAPY

(71) Applicants: The University of Hong Kong, Hong Kong (CN); Laboratory For Synthetic Chemistry and Chemical Biology Limited, Hong Kong (CN)

(72) Inventors: Chi-Ming Che, Hong Kong (CN); Penghe Zhao, Hong Kong (CN); Zhou Tang, Hong Kong (CN)

(21) Appl. No.: 18/927,499

(22) Filed: Oct. 25, 2024

Related U.S. Application Data
(60) Provisional application No. 63/593,831, filed on Oct. 27, 2023.

(51) Int. Cl. C07F 15/02; A61K 33/26; A61N 5/067

(52) U.S. Cl. CPC C07F 15/02; A61K 33/26; A61N 5/067

(57) ABSTRACT

Cyclometalated iron (II) complexes as photothermal transduction agents are described herein. The disclosed complexes show high structural robustness and significant absorption in the near-infrared (NIR) and visible regions. The described complexes can self-assemble to form metallosupramolecular particles, which have excellent photothermal performance and can target tumor by EPR effect. For example, the Fe NPs disclosed herein have strong near-infrared (NIR) absorbance with high photo-heat conversion efficiency of at least 30% (such as about 60%) and/or superior photothermal stability under near-infrared (e.g., 808 nm) laser irradiation. The Fe NPs may be coated with a coating agent, such as bovine serum albumin, to form a coated Fe NPs, which can further enhance the tumor accumulations and biocompatibility of the metallosupramolecular particles in vivo. The excellent photothermal performance of the Fe NPs allow them to solve the problem of low photothermal conversion efficiency associated with most existing photothermal materials.

### CROSS REFERENCE TO RELATED APPLICATION

This application claims priority to U.S. Provisional Patent Application No. 63/593,831 filed Oct. 27, 2023, the disclosure of which is incorporated herein by reference.

### FIELD OF THE INVENTION

This invention is generally in the field of iron (II) complexes, particles, such as metallosupramolecular particles, formed therefrom, and methods of use thereof.

### BACKGROUND OF THE INVENTION

Traditional treatments including chemotherapy, radiotherapy or surgery are always accompanied with sever side effect. Unlike traditional therapy, photothermal therapy (PTT) causes less damage to normal tissues surrounding the tumors due to the poor absorption of skin, tissues, and hemoglobin in the near infrared (NIR) region. Basically, a PTT agent can convert absorbed light energy into heat energy to induce cellular thermal effects, such as protein denaturation cell, mitochondria damage or membrane destruction, thereby ablation of cancer cells. It is also important for PTT reagents to have good photothermal properties, photothermal stability, and biocompatibility and biosafety. Organic dyes, polymeric materials, and inorganic nanoparticles have been extensively explored as potential photosensitizers (PSs) for this purpose. However, despite their photothermal performance, their applications are limited due to the poor photothermal stability, unsatisfactory biosafety and potential long-term damage. Further, accurate imaging is typically required to provide tumor location and monitor nanoparticles accumulation in tumor tissue. Such information is helpful in identifying the best time for treatment. Therefore, developing a new PTT agent is urgent for anticancer research and application.

Recent years, transition metal complexes have made remarkable achievements in the clinical treatment of neuropathy, tumor therapy and diabetes. These putative systems should display good biosafety profiles, benefit from their high stability, light resistant and heat tolerant. They should also permit concurrent imaging of targeted disease states. Fe polypyridine complexes are generally non-luminescent or show extremely short-lived charge-transfer (CT) excited states, which limits their use as alternatives or supplements to heavy metal (e.g., Ir, Ru, Pt ct.) complexes in photo-physical/chemical applications. No molecular Fe(II)-BQ-Bphen₂ complexes have been reported for photothermal therapy studies, even though Fe is an ideal element due to its high abundance and biocompatibility. The scarcity of such reports with Fe(II)-BQ-Bphen₂ complexes is thought to be associated with their general structural instability, especially in bio-environment, and low absorption profile in the low-energy-visible or NIR region.

There remains a need to develop iron (II) complexes as photosensitizers for photothermal therapy.

Therefore, it is the object of the present invention to provide iron (II) complexes.

It is a further object of the present invention to provide particles (e.g., metallosupramolecular particles) formed by the iron (II) complexes.

It is a further object of the present invention to provide methods of using the iron (II) complexes and particles (e.g., metallosupramolecular particles) formed therefrom in photothermal therapy, such as for treatment of cancer.

### SUMMARY OF THE INVENTION

Cyclometalated iron (II) complexes (also referred to herein as “complexes” or “Fe(II) complexes”) as photothermal transduction agents are described herein. The disclosed complexes show high structural robustness owing to the strengthened Fe-L bonds. Further, the disclosed complexes have a narrowed energy gap arising from higher metal-based HOMOs, which allow them to have significant absorption in the near-infrared (NIR) and visible regions (such as confirmed by using UV-vis spectrum).

The described complexes can self-assemble to form metallosupramolecular particles (also referred to herein as “Fe nanoparticles” or “Fe NPs”), such as via non-covalent interactions. The Fe NPs formed from the complexes have excellent photothermal performance and can target tumor by EPR effect. For example, the Fe NPs disclosed herein have strong near-infrared (NIR) absorbance with high photo-heat conversion efficiency of at least 30% (such as about 60%), which is significantly higher than commercial gold nanorods (21.0%), Cu2-xSe (22%), and Cu₉S₅ (25.7%); and/or superior photothermal stability under near-infrared (e.g., 808 nm) laser irradiation (e.g., decrease of photo-heat conversion efficiency is less than 10% for at least 4 cycles of NIR irradiation).

In some forms, the Fe NPs are coated with a coating agent, such as bovine serum albumin (BSA), to form a coated Fe NPs. The coating agent may be associated with the Fe NPs via any suitable interactions, such as electrostatic interactions. Coating with the coating agent can further enhance the tumor accumulations and biocompatibility of the metallosupramolecular particles in vivo, such as shown by the photothermal effect in mice treated with coated Fe NPs, compared to mice treated with Fe NPs without coating, under an 808 nm laser irradiation for a suitable period of time (e.g., about 5 mins, about 6 mins, about 10 mins, about 20 mins, etc.). For example, the coated Fe NPs shows an in vivo photothermal effect (e.g., mice treated with the coated Fe NPs, under an 808 nm laser irradiation for about 5 to about 20 mins) that is at least 1.5-time, at least 2-time, from 1.5-time to 10-time, from 1.5-time to 5-time, or from 1.5-time to 3-time stronger than the in vivo photothermal effect of Fe NPs without coating (e.g., mice treated with the Fe NPs without coating, at the same dose and under the same laser irradiation for the same time period as the mice treated with the coated Fe NPs). See, e.g., FIGS. 13F and 13G described in the Examples below. The excellent photothermal performance of the Fe NPs allow them to solve the problem of low photothermal conversion efficiency associated with most existing photothermal materials (such as those described above).

Methods of using the Fc NPs and coated Fe NPs in photothermal therapy for treating cancer are also disclosed. Generally, the method includes a step of administering a composition comprising the Fe NPs and/or coated Fe NPs to a subject in need thereof. Following the administration step, a laser irradiation is applied to the subject or a target region of the subject that is in need of treatment. Typically, following the laser irradiation step, a tumor in the subject is reduced, in volume and/or weight, by at least 30% compared to the tumor before treatment.

### BRIEF DESCRIPTION OF THE DRAWINGS

FIGS. 1A-1E show the X-ray structures of complex C (FIG. 1A), complex J (FIG. 1B), complex P (FIG. 1C), complex Q (FIG. 1D), and complex S (FIG. 1E). The counter ion and hydrogen atoms were removed for clarity.

FIG. 2 shows the UV-vis absorption spectra of complexes C-G in CH₃CN.

FIG. 3 shows the UV-vis absorption spectra of complexes H-K in CH₃CN.

FIGS. 4A-4D show the UV-vis absorption spectra of complex O in CH₃CN (FIG. 4A) and complex Q in CH₃CN (FIG. 4B), complex R in DCM (FIG. 4C), and complex S in CH₃CN (FIG. 4D).

FIG. 5 shows the cyclic voltammograms of complexes C—O in CH₃CN.

FIG. 6 shows the cyclic voltammogram of complex Q in DCM.

FIG. 7A shows the fs-TA spectra of complex F recorded with 455 nm excitation in MeCN. FIG. 7B shows the kinetic profiles of the TA intensity at 470 nm wavelength.

FIG. 8A shows the fs-TA spectra of complex C recorded with 266 nm excitation in MeCN. FIG. 8B shows the kinetic profiles of the TA intensity at 420 nm wavelength. FIG. 8C shows the fs-TA spectra of complex K in MeCN. FIG. 8D shows the kinetic fitting at 595 nm for complex K.

FIGS. 9A and 9B show the fs-TA spectra of complex G recorded with 455 nm excitation in MeCN. FIG. 9C shows the kinetic profiles of the TA intensity at 500 nm wavelength.

FIG. 10 shows the differential absorption spectrum of complex C upon one-electron reduction in with 0.1 M MeCN solution of (TBA)PF₆.

FIG. 11 shows the differential absorption spectrum of complex G upon one-electron reduction in with 0.1 M MeCN solution of (TBA)PF₆.

FIGS. 12A-12E show the characterization of Fe(II)-BQ-Bphen2 complex nanoparticles. FIG. 12A is a schematic showing the nanoparticles preparation process. FIGS. 12B-12E show the UV-vis absorption spectrum of the Fe(II)-BQ-Bphen2 complex nanoparticles (FIG. 12B), dynamic light scattering (DLS) of the Fe(II)-BQ-Bphen2 complex nanoparticles in H₂O (FIG. 12C), zeta potential of Fe(II)-BQ-Bphen2 complex nanoparticles (FIG. 12D), and TEM image of Fe(II)-BQ-Bphen2 complex nanoparticles (FIG. 12E, top two images) and Fe(II)-BQ-Bphen2 complex nanoparticles coated with BSA (FIG. 12E, bottom two images). FIG. 12F shows the UV-vis absorption change of Fe NPs (without coating) with time in pH 7.4 PBS. FIG. 12G is a graph showing the stability of the Fe NPs with time based on the UV-vis data shown in FIG. 12F. FIG. 12H shows the UV-vis absorption change of BSA coated Fe NPs with time in pH 7.4 PBS. FIG. 12I is a graph showing the stability of the BSA coated Fe NPs with time based on the UV-vis data shown in FIG. 12H.

FIGS. 13A-13E show the photothermal effect of the Fe(II)-BQ-Bphen2 complex nanoparticles. FIG. 13A is a graph showing the temperature elevation under three power density of near infrared laser irradiation (808 nm, 0.2 0.5 and 1 W/cm²) in the concentration of 100 μg/mL. FIG. 13B is a graph showing the temperature elevation with different concentrations of the Fe(II)-BQ-Bphen2 complex nanoparticles after 808 nm laser (1.0 W/cm) irradiation for 12 min. FIG. 13C is a graph showing the steady-state heating curves of the Fe(II)-BQ-Bphen2 complex nanoparticles in aqueous solution, irradiated with laser (100 μg/mL, 1.0 W/cm). The photothermal effect of Fe NPs aqueous dispersion (100 ppm) under irradiation of an 808 laser with the power density of 1 W cm 2 and the laser was turned off after irradiation for 22 min. The time constant for heat transfer from the system was determined to be τs=473.13 s by applying the linear time data from the cooling period (after 22 min) versus negative natural logarithm of the driving force temperature obtained from the cooling stage of the curve. FIG. 13D is a graph showing temperature change of nanoparticles solution during 4 irradiation and cooling cycles (100 μg/mL, 1 W/cm²). FIG. 13E is a graph showing the photothermal curve of tumors on mice during 808 nm laser irradiation 6 min after 1 h via intravenous tail vein injection of PBS, Fc(II)-BQ-Bphen2 complex nanoparticles (10 mg/kg), or BSA coated Fe(II)-BQ-Bphen2 complex nanoparticles (10 mg/kg). FIG. 13F shows the photothermal images of tumors on the mice treated with PBS, Fc(II)-BQ-Bphen2 complex nanoparticles (10 mg/kg), or BSA coated Fc(II)-BQ-Bphen2 complex nanoparticles (10 mg/kg). These images, from top to bottom, correspond with the temperature data of the photothermal curves from top to bottom shown in FIG. 13E.

FIGS. 14A-14F show the in vitro phototherapy efficiencies and cellular uptake of Fc-NPs. FIGS. 14A-14B show the Fe-NPs-dose dependent cell viabilities in 4T1 cells (FIG. 14A) and NCI H460 cells (FIG. 14B) in the presence or absence of 808 nm laser irradiation, mean±SD (n=4). Unpaired, two-sided t tests *p<0.05, **p<0.01, and ***p<0.001. FIGS. 14C-14D show the high-content microscopy counts of dead cells and whole cells using propidium iodide and Hoechst stain assay in 4T1 cells (FIG. 14C) and NCI H460 cells (FIG. 14D). FIG. 14E is a graph showing the fluorescence intensity of ROS generation in 4T1 cells under different treatment conditions by using a microplate reader (Ex 490 nm, Em 525 nm). Irradiation was performed using a 808 nm laser at 1 W/cm² for 10 min. FIG. 14F is a graph showing the flow cytometry analysis for apoptosis of 4T1 cells treated with vehicle, Fe-NPs (25 μM, 50 μM and 100 μM) with or without 808 nm laser irradiation (1 W/cm², 10 min).

FIGS. 15A-15C are graphs showing the anti-tumor effect of Fe NPs. FIG. 15A is a schematic showing the approach of 4T1 tumor-bearing mice treatment. FIG. 15B is a graph showing the tumor volume of nude mice bearing 4T1 xenograft change during treatment with PBS, Fe-NPs (10 mg/kg) via intravenous tail vain injection of Fe NPs, followed by tumor irradiation at 808 nm (0.5 W/cm²) for 6 min. FIG. 15C is a graph showing the 4T1 tumor weight comparison after 16-day treatment and the digital images of corresponding 4T1 tumors (n=5). Mean value and error bar are defined as mean and s.d., respectively. P values were calculated by two-tailed Student's t-test (***P<0.005, *P<0.05) by comparing other groups with the Fe-NPs with laser irradiation.

FIGS. 16A-16O are bar graphs showing the evaluation of standard haematology markers and blood biochemical analyses: white blood cells (WBC) (FIG. 16A), red blood cells (RBC) (FIG. 16B), hemoglobin (HGB) (FIG. 16C), cran (FIG. 16D), mcv (FIG. 16E), rdw-cv (FIG. 16F), HCT (FIG. 16G), Lymph (FIG. 16H), PLT (FIG. 16I), PCT (FIG. 16J), ALT (FIG. 16K), AST (FIG. 16L), ALP (FIG. 16M), Bun (FIG. 16N), Crea (FIG. 16O).

FIGS. 17A-17F are graphs showing the mild PTT-induced immune responses. FIG. 17A shows the approach of 4T1 tumor-bearing mice treatment with mild power 808 nm laser treatment (0.3 W/cm²). FIG. 17B shows the weight changes of mice under different treatment. FIGS. 17C and 17D are bar graphs showing DC maturation (CD80 expression: FIG. 17C; CD86 expression: FIG. 17D) induced by mild PTT on 4T1 tumor-bearing mice gated on CD11+DC cells. Cells in the tumors were collected on day 15 after various treatments for assessment by flow cytometry after staining with CD11c+, CD80, and CD86. Data represents mean±SEM (n=6 biologically independent samples). FIGS. 17E and 17F are bar graphs showing the quantification of CD4 (FIG. 17E) and CD8 (FIG. 17F) based on flow cytometry intensity. The comparison of two groups was followed by unpaired student's t-test (two-tailed). *P<0.05, **P<0.01, and ***P<0.001.

### DETAILED DESCRIPTION OF THE INVENTION

### I. Definitions

It is to be understood that the disclosed compounds, compositions, and methods are not limited to specific synthetic methods, specific analytical techniques, or to particular reagents unless otherwise specified, and, as such, may vary. It is also to be understood that the terminology used herein is for the purpose of describing particular forms and embodiments only and is not intended to be limiting.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid. Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, and an amino acid can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

“Alkyl,” as used herein, refers to the radical of saturated aliphatic groups, including straight-chain alkyl groups, branched-chain alkyl, and cycloalkyl (alicyclic). In some forms, a straight chain or branched chain alkyl has 30 or fewer carbon atoms in its backbone (e.g., C₁-C₃₀ for straight chains, C₃-C₃₀ for branched chains), 20 or fewer, 15 or fewer, or 10 or fewer. Alkyl includes methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Likewise, a cycloalkyl is a non-aromatic carbon-based ring composed of at least three carbon atoms, such as a nonaromatic monocyclic or nonaromatic polycyclic ring containing 3-30 carbon atoms, 3-20 carbon atoms, or 3-10 carbon atoms in their ring structure, and have 5, 6 or 7 carbons in the ring structure. Cycloalkyls containing a polycyclic ring system can have two or more non-aromatic rings in which two or more carbons are common to two adjoining rings (i.e., “fused cycloalkyl rings”). Examples of cycloalkyl groups include, but are not limited to, cyclopropyl, cyclobutyl, cyclopentyl, cyclohexyl, cycloheptyl, cyclooctanyl, etc.

“Substituted alkyl” refers to alkyl moieties having one or more substituents replacing a hydrogen on one or more carbons of the hydrocarbon backbone. Such substituents can be any substituents described above, e.g., halogen (such as fluorine, chlorine, bromine, or iodine), hydroxyl, carbonyl (such as a carboxyl, alkoxycarbonyl, formyl, or an acyl), thiocarbonyl (such as a thioester, a thioacetate, or a thioformate), aryl, alkoxyl, aralkyl, phosphonium, phosphanyl, phosphonyl, phosphoryl, phosphate, phosphonate, a phosphinate, amino, amido, amidine, imine, cyano, nitro, azido, oxo, sulfhydryl, thiol, alkylthio, silyl, sulfinyl, sulfate, sulfonate, sulfamoyl, sulfonamido, sulfonyl, heterocyclyl, an aromatic or heteroaromatic moiety. —NRR′, wherein R and R′ are independently hydrogen, alkyl, or aryl, and wherein the nitrogen atom is optionally quaternized; —SR, wherein R is a phosphonyl, a sulfinyl, a silyl a hydrogen, an alkyl, or an aryl; —CN; —NO₂; —COOH; carboxylate; —COR, —COOR, or —CON(R)₂, wherein R is hydrogen, alkyl, or aryl; imino, silyl, ether, haloalkyl (such as —CF₃, —CH₂—CF₃, —CCl₃); —CN; —NCOCOCH₂CH₂; —NCOCOCHCH; and —NCS; and combinations thereof.

It will be understood by those skilled in the art that the moieties substituted on the hydrocarbon chain can themselves be substituted, if appropriate. For instance, the substituents of a substituted alkyl may include halogen, hydroxy, nitro, thiols, amino, aralkyl, azido, imino, amido, phosphonium, phosphanyl, phosphoryl (including phosphonate and phosphinate), oxo, sulfonyl (including sulfate, sulfonamido, sulfamoyl and sulfonate), and silyl groups, as well as ethers, alkylthios, carbonyls (including ketones, aldehydes, carboxylates, and esters), haloalkyls, —CN and the like. Cycloalkyls can be substituted in the same manner.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths.

“Heteroalkyl,” as used herein, refers to straight or branched chain, or cyclic carbon-containing alkyl radicals, or combinations thereof, containing at least one heteroatom on the carbon backbone. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “alkenyl” as used herein is a hydrocarbon group of from 2 to 24 carbon atoms and structural formula containing at least one carbon-carbon double bond. Alkenyl groups include straight-chain alkenyl groups, branched-chain alkenyl, and cycloalkenyl. A cycloalkenyl is a non-aromatic carbon-based ring composed of at least three carbon atoms and at least one carbon-carbon double bond, such as a nonaromatic monocyclic or nonaromatic polycyclic ring containing 3-30 carbon atoms and at least one carbon-carbon double bond, 3-20 carbon atoms and at least one carbon-carbon double bond, or 3-10 carbon atoms and at least one carbon-carbon double bond in their ring structure, and have 5, 6 or 7 carbons and at least one carbon-carbon double bond in the ring structure. Cycloalkenyls containing a polycyclic ring system can have two or more non-aromatic rings in which two or more carbons are common to two adjoining rings (i.e., “fused cycloalkenyl rings”) and contain at least one carbon-carbon double bond. Asymmetric structures such as (AB)C═C(C′D) are intended to include both the/and Z isomers. This may be presumed in structural formulae herein wherein an asymmetric alkene is present, or it may be explicitly indicated by the bond symbol C. The term “alkenyl” as used throughout the specification, examples, and claims is intended to include both “unsubstituted alkenyls” and “substituted alkenyls,” the latter of which refers to alkenyl moieties having one or more substituents replacing a hydrogen on one or more carbons of the hydrocarbon backbone. The term “alkenyl” also includes “heteroalkenyl.”

The term “substituted alkenyl” refers to alkenyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, oxo, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkenyl,” as used herein, refers to straight or branched chain, or cyclic carbon-containing alkenyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkenyl group” is a cycloalkenyl group where at least one of the carbon atoms of the ring is substituted with a heteroatom such as, but not limited to, nitrogen, oxygen, sulphur, or phosphorus.

The term “alkynyl group” as used herein is a hydrocarbon group of 2 to 24 carbon atoms and a structural formula containing at least one carbon-carbon triple bond. Alkynyl groups include straight-chain alkynyl groups, branched-chain alkynyl, and cycloalkynyl. A cycloalkynyl is a non-aromatic carbon-based ring composed of at least three carbon atoms and at least one carbon-carbon triple bond, such as a nonaromatic monocyclic or nonaromatic polycyclic ring containing 3-30 carbon atoms and at least one carbon-carbon triple bond, 3-20 carbon atoms and at least one carbon-carbon triple bond, or 3-10 carbon atoms and at least one carbon-carbon triple bond in their ring structure, and have 5, 6 or 7 carbons and at least one carbon-carbon triple bond in the ring structure. Cycloalkynyls containing a polycyclic ring system can have two or more non-aromatic rings in which two or more carbons are common to two adjoining rings (i.e., “fused cycloalkynyl rings”) and contain at least one carbon-carbon triple bond. Asymmetric structures such as (AB)C≡C(C″D) are intended to include both the E and Z isomers. This may be presumed in structural formulae herein wherein an asymmetric alkyne is present, or it may be explicitly indicated by the bond symbol C. The term “alkynyl” as used throughout the specification, examples, and claims is intended to include both “unsubstituted alkynyls” and “substituted alkynyls,” the latter of which refers to alkynyl moieties having one or more substituents replacing a hydrogen on one or more carbons of the hydrocarbon backbone. The term “alkynyl” also includes “heteroalkynyl.”

The term “substituted alkynyl” refers to alkynyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkynyl,” as used herein, refers to straight or branched chain, or cyclic carbon-containing alkynyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkynyl group” is a cycloalkynyl group where at least one of the carbon atoms of the ring is substituted with a heteroatom such as, but not limited to, nitrogen, oxygen, sulphur, or phosphorus.

“Aryl,” as used herein, refers to C₄-C₂₆-membered aromatic rings or fused ring systems containing one aromatic ring and optionally one or more non-aromatic rings. Examples of aryl groups are benzene, tetralin, indane, etc.

The term “substituted aryl” refers to an aryl group, wherein one or more hydrogen atoms on one or more aromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

“Heterocyclo” and “heterocyclyl” are used interchangeably, and refer to a cyclic radical attached via a ring carbon or nitrogen atom of a monocyclic ring or polycyclic ring system containing 3-30 ring atoms, 3-20 ring atoms, 3-10 ring atoms, or 5-6 ring atoms, where the polycyclic ring system contains one or more non-aromatic rings and optionally one or more aromatic rings, where at least one non-aromatic ring contains carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Heterocycles can be a heterocycloalkyl, a heterocycloalkenyl, a heterocycloalkynyl, etc, such as piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₃-C₂₆-membered aromatic rings or fused ring systems containing one aromatic ring and optionally one or more non-aromatic rings, in which one or more carbon atoms on the aromatic ring structure have been substituted with a heteroatom. Suitable heteroatoms include, but are not limited to, oxygen, sulfur, and nitrogen. Examples of heteroaryl groups pyrrole, furan, thiophene, imidazole, oxazole, thiazole, triazole, tetrazole, pyrazole, pyridine, pyrazine, pyridazine and pyrimidine, and the like. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl.”

The term “substituted heteroaryl” refers to a heteroaryl group in which one or more hydrogen atoms on one or more heteroaromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

The term “polyaryl” refers to a fused ring system that includes two or more aromatic rings and optionally one or more non-aromatic rings. Examples of polyaryl groups are naphthalene, anthracene, phenanthrene, chrysene, pyrene, corannulene, coronene, etc. When a fused ring system containing two or more aromatic rings and optionally one or more non-aromatic rings, in which one or more carbon atoms on two or more aromatic ring structures have been substituted with a heteroatom, the fused ring system can be referred to as a “polyheteroaryl”. When a fused ring system containing two or more aromatic rings and optionally one or more non-aromatic rings, in which one or more carbon atoms in the fused ring system is substituted with a heteroatom it can be referred to as a “heteropolyaryl.”

The term “substituted polyaryl” refers to a polyaryl in which one or more of the aryls are substituted, with one or more substituents including, but not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof. When a polyheteroaryl is involved, the chemical moiety can be referred to as a “substituted polyheteroaryl.”

The term “cyclic,” “cyclic ring” or “cyclic group” refers to a substituted or unsubstituted monocyclic ring or a substituted or unsubstituted polycyclic ring (such as those formed from single or fused ring systems), such as a substituted or unsubstituted cycloalkyl, a substituted or unsubstituted cycloalkenyl, a substituted or unsubstituted cycloalkynyl, or a substituted or unsubstituted heterocyclyl, that have from three to 30 carbon atoms, as geometric constraints permit. The substituted cycloalkyls, cycloalkenyls, cycloalkynyls, and heterocyclyls are substituted as defined above for the alkyls, alkenyls, alkynyls, and heterocyclyls, respectively.

The term “aralkyl” as used herein is an aryl group or a heteroaryl group having an alkyl, alkynyl, or alkenyl group as defined above attached to the aromatic group, such as an aryl, a heteroaryl, a polyaryl, or a polyheteroaryl. An example of an aralkyl group is a benzyl group.

The terms “alkoxyl” or “alkoxy,” “aroxy” or “aryloxy,” generally describe compounds represented by the formula —OR^{v}, wherein R^{v} includes, but is not limited to, a substituted or unsubstituted alkyl, a substituted or unsubstituted alkenyl, a substituted or unsubstituted alkynyl, a substituted or unsubstituted cycloalkyl, a substituted or unsubstituted heterocyclyl, a substituted or unsubstituted cycloalkenyl, a substituted or unsubstituted heterocycloalkenyl, a substituted or unsubstituted aryl, a substituted or unsubstituted heteroaryl, a substituted or unsubstituted polyaryl, a substituted or unsubstituted polyheteroaryl, a substituted or unsubstituted arylalkyl, a substituted or unsubstituted heteroalkyl, a substituted or unsubstituted alkylaryl, a substituted or unsubstituted alkylheteroaryl, a substituted or unsubstituted aralkyl, a substituted or unsubstituted carbonyl, a phosphonium, a phosphanyl, a phosphonyl, a sulfinyl, a silyl, a thiol, an amido, and an amino. Exemplary alkoxyl groups include methoxy, ethoxy, propyloxy, tert-butoxy and the like. A “lower alkoxy” group is an alkoxy group containing from one to six carbon atoms. An “ether” is two functional groups covalently linked by an oxygen as defined below. Accordingly, the substituent of an alkyl that renders that alkyl an ether is or resembles an alkoxyl, such as can be represented by one of —O-alkyl, —O-alkenyl, —O-alkynyl, —O-arakyl, —O-aryl, —O-heteroaryl, —O-polyaryl, —O-polyheteroaryl, —O-heterocyclyl, etc.

The term “substituted alkoxy” refers to an alkoxy group having one or more substituents replacing one or more hydrogen atoms on one or more carbons of the alkoxy backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, oxo, sulfate, sulfonate, sulfamoyl, sulfonamido, sulfonyl, heterocyclyl, alkylaryl, haloalkyl, —CN, aryl, heteroaryl, and combinations thereof.

The term “ether” as used herein is represented by the formula A²OA¹, where A² and A¹ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a phosphonyl, a sulfinyl, a silyl, a thiol, a substituted or unsubstituted carbonyl, an alkoxy, an amido, or an amino, described above.

The term “polyether” as used herein is represented by the formula:

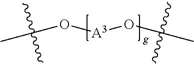

where A³ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a substituted or unsubstituted carbonyl, an alkoxy, an amido, or an amino, described above; g can be a positive integer from 1 to 30.

The term “phenoxy” is art recognized and refers to a compound of the formula —OR^{v} wherein R^{v} is C₆H₅ (i.e., —O—C₆H₅). One of skill in the art recognizes that a phenoxy is a species of the aroxy genus.

The term “substituted phenoxy” refers to a phenoxy group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The terms “aroxy” and “aryloxy,” as used interchangeably herein, are represented by —O-aryl or —O-heteroaryl, wherein aryl and heteroaryl are as defined herein.

The terms “substituted aroxy” and “substituted aryloxy,” as used interchangeably herein, represent —O-aryl or —O-heteroaryl, having one or more substituents replacing one or more hydrogen atoms on one or more ring atoms of the aryl and heteroaryl, as defined herein. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “amino” as used herein includes the group

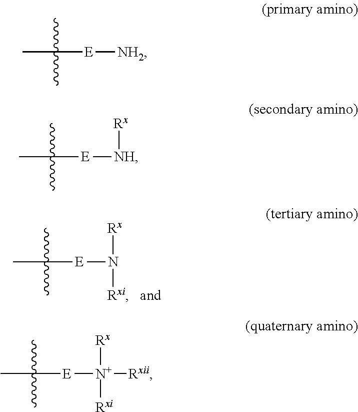

wherein, E is absent, or E is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl, wherein independently of E, R^{x}, R^{xi}, and R^{xii} each independently represent a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. The term “quaternary amino” also includes the groups where the nitrogen, R^{x}, R^{xi}, and R^{xii} with the N⁺ to which they are attached complete a heterocyclyl or heteroaryl having from 3 to 14 atoms in the ring structure. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The terms “amide” or “amido” are used interchangeably, refer to both “unsubstituted amido” and “substituted amido” and are represented by the general formula:

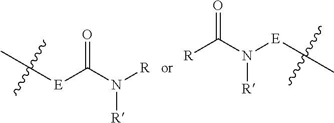

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted aryl, substituted or unsubstituted heteroaryl, a substituted or unsubstituted polyaryl, a substituted or unsubstituted polyheteroaryl, or a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. In some forms, when E is oxygen, a carbamate is formed. It is understood by those of ordinary skill in the art, that the E groups listed above are divalent (e.g., methylene, ethane-1,2-diyl, ethene-1,2-diyl, 1,4-phenylene, cyclohexane-1,2-diyl).

“Carbonyl,” as used herein, is art-recognized and includes such moieties as can be represented by the general formula:

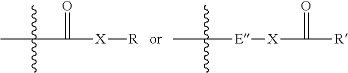

wherein X is a bond, or represents an oxygen or a sulfur, and R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″, or a pharmaceutical acceptable salt; E″ is absent, or E″ is a substituted or unsubstituted alkyl, a substituted or unsubstituted alkenyl, a substituted or unsubstituted alkynyl, a substituted or unsubstituted aralkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl; R′ represents a hydrogen, a substituted or unsubstituted alkyl, a substituted or unsubstituted alkenyl, a substituted or unsubstituted alkynyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″; R″ represents a hydroxyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof. It is understood by those of ordinary skill in the art, that the E″ groups listed above are divalent (e.g., methylene, ethane-1,2-diyl, ethene-1,2-diyl, 1,4-phenylene, cyclohexane-1,2-diyl). Where X is oxygen and R is defined as above, the moiety is also referred to as a carboxyl group. When X is oxygen and R is hydrogen, the formula represents a “carboxylic acid.” Where X is oxygen and R′ is hydrogen, the formula represents a “formate.” Where X is oxygen and R or R′ is not hydrogen, the formula represents an “ester.” In general, where the oxygen atom of the above formula is replaced by a sulfur atom, the formula represents a “thiocarbonyl” group. Where X is sulfur and R or R′ is not hydrogen, the formula represents a “thioester.” Where X is sulfur and R is hydrogen, the formula represents a “thiocarboxylic acid.” Where X is sulfur and R′ is hydrogen, the formula represents a “thioformate.” Where X is a bond and R is not hydrogen, the above formula represents a “ketone.” Where X is a bond and R is hydrogen, the above formula represents an “aldehyde.”

The term “phosphanyl” is represented by the formula

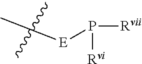

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi} and R^{vii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphonium” is represented by the formula

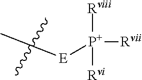

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi}, R^{vii}, and R^{viii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi}, R^{vii}, and R^{viii} taken together with the P⁺ atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphonyl” is represented by the formula

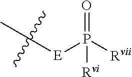

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, oxygen, alkoxy, aroxy, or substituted alkoxy or substituted aroxy, wherein, independently of E, R^{vi} and R^{vii} are independently a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphoryl” defines a phosphonyl in which E is absent, oxygen, alkoxy, aroxy, substituted alkoxy or substituted aroxy, as defined above, and independently of E, R^{vi} and R^{vii} are independently hydroxyl, alkoxy, aroxy, substituted alkoxy or substituted aroxy, as defined above. When E is oxygen, the phosphoryl cannot be attached to another chemical species, such as to form an oxygen-oxygen bond, or other unstable bonds, as understood by one of ordinary skill in the art. When E, R^{vi} and R^{vii} are substituted, the substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfinyl” is represented by the formula

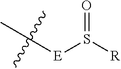

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a silyl, a thiol, an amido, an amino, or —(CH₂) m-R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonyl” is represented by the formula

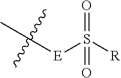

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, a substituted or unsubstituted polyaryl, a substituted or unsubstituted polyheteroaryl, or substituted or unsubstituted heteroaryl. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amino, substituted or unsubstituted cycloalkyl, substituted or unsubstituted heterocyclyl, substituted or unsubstituted aralkyl, substituted or unsubstituted alkylaryl, substituted or unsubstituted arylalkyl, substituted or unsubstituted aryl, substituted or unsubstituted heteroaryl, a substituted or unsubstituted polyaryl, a substituted or unsubstituted polyheteroaryl, —(CH₂)ₘ—R′″, R′″ represents a hydroxy group, substituted or unsubstituted carbonyl group, an aryl, a cycloalkyl ring, a cycloalkenyl ring, a heterocycle, an amido, an amino, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfamoyl” refers to a sulfonamide or sulfonamide represented by the formula

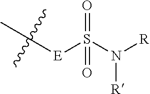

wherein E is absent, or E is substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted cycloalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “silyl group” as used herein is represented by the formula —SiRR′R,” where R, R′, and R″ can be, independently, a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, a phosphonyl, a sulfinyl, a thiol, an amido, an amino, an alkoxy, or an oxo, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The terms “thiol” are used interchangeably and are represented by —SR, where R can be a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, an amido, an amino, an alkoxy, an oxo, a phosphonyl, a sulfinyl, or a silyl, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted, independently, with the substituents described above in the definition of “substituted.”

The numerical ranges disclose individually each possible number that such a range could reasonably encompass, as well as any sub-ranges and combinations of sub-ranges encompassed therein. For example, in a given range carbon range of C₃-C₉, the range also discloses C₃, C₄, C₅, C₆, C₇, C₈, and C₉, as well as any subrange between these numbers (for example, C₄-C₆), and any possible combination of ranges possible between these values. In yet another example, a given temperature range may be from about 25° C. to 30° C., where the range also discloses temperatures that can be selected independently from about 25, 26, 27, 28, 29, and 30° C., as well as any range between these numbers (for example, 26 to 28° C.), and any possible combination of ranges between these values.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted with, independently, with the substituents described above in the definition of “substituted.”

### II. Compositions

Cyclometalated iron (II) complexes (also referred to herein as “complexes,” “Fe(II) complexes,” or “compounds”) and particles formed from these complexes have been developed. The complexes and particles formed therefrom are suitable for use as photothermal transduction agents. The disclosed complexes show high structural robustness and significant absorption in the near-infrared (NIR) and/or visible regions (e.g., absorption at wavelength of at least 670 nm), such as confirmed by using UV-vis spectrum. Without being bound to any theories, it is believed that the high structural robustness of the disclosed complexes is owing to the strengthened Fe-L bonds, and the absorption in the NIR and/or visible regions is owing to a narrowed energy gap arising from higher metal-based HOMOs. Further, the disclosed complexes have a ³MLCT excited state lifetime (also referred to herein as “excited state lifetime” or “lifetime”) of at least 1 ps, measured using fs-TA spectroscopy in combination with UV-vis spectroelectrochemistry. In some forms, the lifetime of the disclosed complexes is at least 2 orders higher than those of traditional polypyridine Fe(II) complexes, such as about 2 orders higher than those of traditional polypyridine Fe(II) complexes. The long ³MLCT excited-state lifetime (e.g., approaching ns regime) of the disclosed Fe(II) complexes is desirable for photocatalytic applications involving excited-state electron- or energy-transfer processes. Further, such a lifetime does not exhibit appreciable photoluminescence quantum yields, which is desirable as luminescence does not act as a dominant excited-state decaying pathway that limits the photothermal quantum efficiency. Without being bound to any theories, it is believed that the cyclometallation structure of the disclosed complexes can lead to a strong-field effect. Such a strong ligand field can directly correlate with longer ³MLCT lifetimes relative to those of traditional polypyridine Fe(II) complexes.

Metallosupramolecular particles (also referred to herein as “Fe nanoparticles” or “Fe NPs”) can be formed through self-assembly of the disclosed complexes, such as via non-covalent interactions. The Fe NPs formed from the complexes have excellent photothermal performance and can target tumor by enhanced permeability retention (EPR) effect. For example, the Fe NPs disclosed herein have strong near-infrared (NIR) absorbance with high photo-heat conversion efficiency of at least 30% (such as about 60%, measured using a 808 nm laser irradiation at 1.0 W/cm for about 20 min, such as 22 mins), which is significantly higher than commercial gold nanorods (21.0%), Cu₂-xSe (22%), and Cu₉S₅ (25.7%); and/or superior photothermal stability under near-infrared (e.g., 808 nm) laser irradiation (e.g., decrease of photo-heat conversion efficiency is less than 10% for at least 4 cycles of laser irradiation-cooling, where each laser irradiation lasts at least 5 mins).

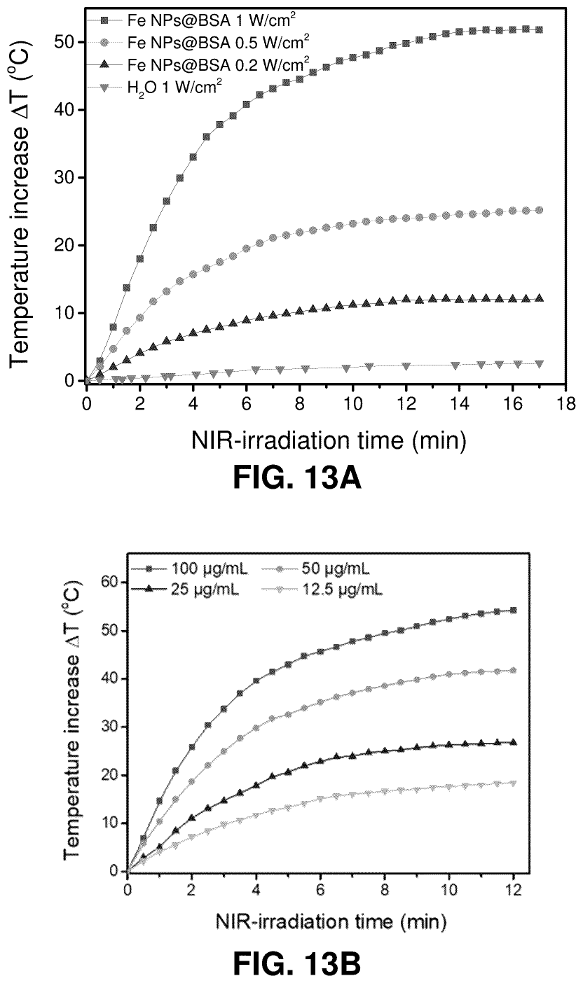

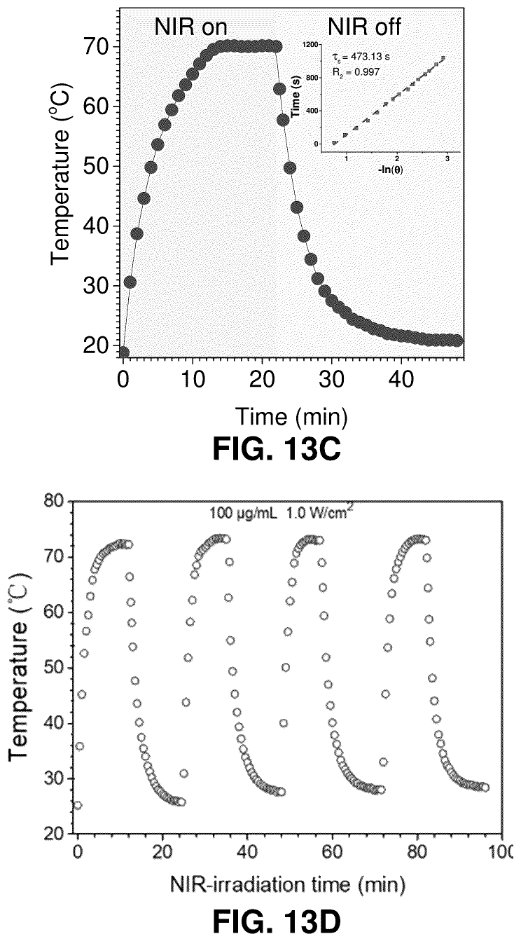

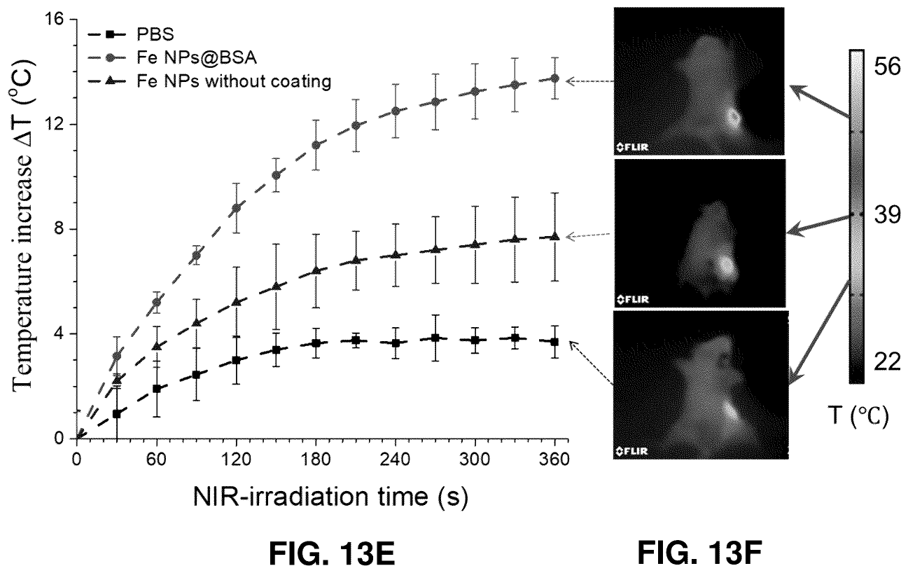

The Fe NPs may be coated with a coating agent, such as bovine serum albumin (BSA), to form a coated Fe NPs. The coating agent may be associated with the Fe NPs via any suitable interactions, covalent or non-covalent, such as electrostatic interactions. Coating with the coating agent can further enhance the tumor accumulations and biocompatibility of the metallosupramolecular particles in vivo, such as shown by the photothermal effect in mice treated with coated Fe NPs, compared to mice treated with Fe NPs without coating, under an 808 nm laser irradiation for a suitable period of time (e.g., about 5 mins, about 6 mins, about 10 mins, about 20 mins, etc.). For example, the coated Fe NPs shows an in vivo photothermal effect (e.g., mice treated with the coated Fe NPs, under an 808 nm laser irradiation for about 5 to about 20 mins) that is at least 1.5-time, at least 2-time, from 1.5-time to 10-time, from 1.5-time to 5-time, or from 1.5-time to 3-time stronger than the in vivo photothermal effect of Fe NPs without coating (e.g., mice treated with the Fe NPs without coating, at the same dose and under the same laser irradiation for the same time period as the mice treated with the coated Fe NPs). See, e.g., FIGS. 13G and 13H described in the Examples below. The excellent photothermal performance of the Fe NPs allow them to solve the problem of low photothermal conversion efficiency associated with most existing photothermal materials (such as those described above).

### A. Iron (II) Complexes

The cyclometalated iron (II) complexes disclosed herein are photothermal agent with high quantum efficiency. The photothermal quantum efficiency of the Fe nanoparticles disclosed herein is high among the reported nanoparticles formed by molecular metal complexes, such as those described in Xu, Y., et al., Nat Commun. 2022 13 (1): 2009; Yao, Y., et al., Journal of the American Chemical Society, 2022 144 (16): 7346-7356. These are suitable for use as photosensitizer in cancer photothermal therapy. The disclosed complexes, when compared with the benchmark [Fe^{II}(bpy)₃]²⁺ complex, show a profound σ-donating effect of the cyclometalating donor, and are stable in air. Further, the disclosed complexes show absorption in the near-infrared and visible region (e.g., from about 200 nm to about 1100 nm, such as from about 600 nm to about 850 nm, from about 600 nm to about 1100 nm, from about 700 nm to about 850 nm, or from about 700 nm to about 1100 nm), and an extended ³MLCT lifetime (e.g., at least 1 ps, suh as about 2.59 ps), which is at least one order longer than the benchmark [Fe(bpy)₃]²⁺ complexes. In some forms, the iron complexes can be monocyclometalated tris(bidentate) [Fe^{II}(ppy)(N\hat{ }N)₂]⁺, [Fe^{I}(BQ)(N\hat{ }N)₂]⁺ (ppy=2-(pyridin-2-yl)benzen-1-ide, BQ=benzo[h]quinolin-10-ide), and [Fe^{II}(ppy)(tpy)Cl](tpy=2,2′:6′,2″-terpyridine) complexes.

In some forms, the disclosed iron (II) complex can have the structure of Formula I or Formula I′:

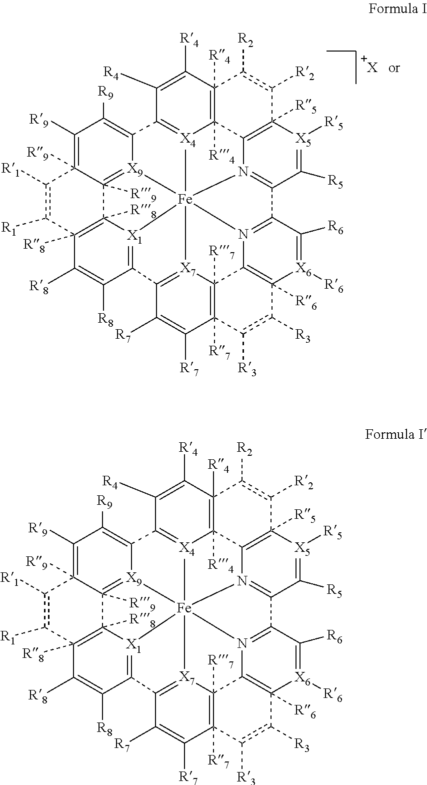

wherein: (i) X₁, X₄, X₅, X₆, X₇, and X₉ can be independently carbon or nitrogen, X can be an anion (e.g., PF₆⁻, Cl⁻, OTf⁻, etc.); (ii) each [symbol] can be absent or a single bond, each [symbol] can be absent or a double bond; (iii) R₁, R′₁, R₂, R′₂, R₃, and R′₃, when present, can be independently hydrogen, substituted or unsubstituted alkyl, substituted or unsubstituted aryl, or substituted or unsubstituted alkylaryl; (iv) R₄, R′₄, R₅, R′₅, R₆, R′₆, R₇, R′₇, R₈, R′₈, R₉, and R′₉ can be independently hydrogen, substituted or unsubstituted alkyl, substituted or unsubstituted aryl, substituted or unsubstituted heteroaryl, substituted or unsubstituted polyaryl, substituted or unsubstituted heteropolyaryl, substituted or unsubstituted alkylaryl, substituted or unsubstituted cyclic, substituted or unsubstituted heterocyclic, halide, amino, amido, thiol, hydroxyl, cyano, nitro, carbonyl, or alkoxyl; (v) R″₄, R′″₄, R″₅, R″₆, R″₇, R′″₇, R″₈, R′″₈, R″₉, and R′″₉, when present, can be independently hydrogen, halide, substituted or unsubstituted alkyl, substituted or unsubstituted aryl, substituted or unsubstituted heteroaryl, substituted or unsubstituted cyclic (e.g., cycloalkyl, cycloalkenyl, cycloalkynyl), or substituted or unsubstituted heterocyclic; and (vi) the substituents, when present, can be independently unsubstituted alkyl, unsubstituted aryl, unsubstituted heteroaryl, unsubstituted polyaryl, unsubstituted heteropolyaryl, unsubstituted alkylaryl, unsubstituted cyclic, unsubstituted heterocyclic, halide, amino, amido, thiol, hydroxyl, cyano, nitro, carbonyl, or alkoxyl.

In some forms, the iron (II) complex can have the structure of Formula II or Formula II′:

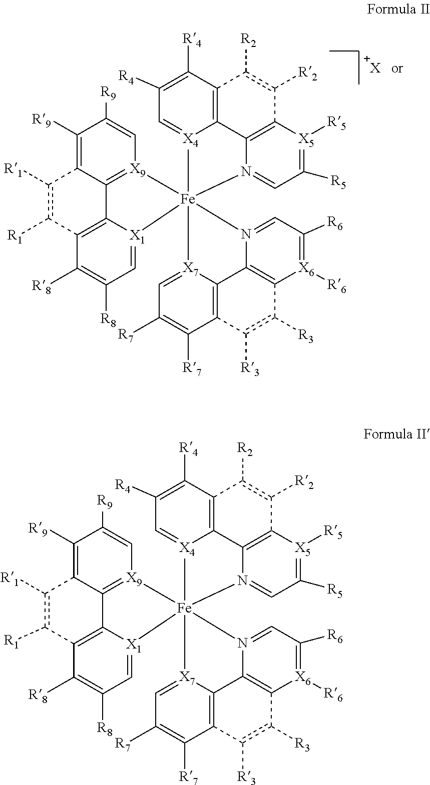

wherein: (i) X₁, X₄, X₅, X₆, X₇, and X₉ can be independently carbon or nitrogen, X can be an anion (e.g., PF₆⁻, Cl⁻, OTf⁻, etc.); (ii) each [symbol] can be absent or a single bond, each [symbol] can be absent or a double bond; (iii) R₁, R′₁, R₂, R′₂, R₃, and R′₃, when present, can be independently hydrogen, substituted or unsubstituted alkyl, or substituted or unsubstituted aryl; (iv) R₄, R′₄, R₅, R′₅, R₆, R′₆, R₇, R′₇, R₈, R′₈, R₉, and R′₉ can be independently hydrogen, substituted or unsubstituted alkyl, substituted or unsubstituted aryl, substituted or unsubstituted heteroaryl, substituted or unsubstituted heterocyclic, or halide; and (v) the substituents, when present, are independently unsubstituted alkyl, unsubstituted aryl, unsubstituted heteroaryl, unsubstituted polyaryl, unsubstituted heteropolyaryl, unsubstituted alkylaryl, unsubstituted cyclic (e.g., cycloalkyl, cycloalkenyl, cycloalkynyl), unsubstituted heterocyclic, halide, amino, amido, thiol, hydroxyl, cyano, nitro, carbonyl, or alkoxyl.

In some forms, the iron (II) complex can have the structure of Formula III or Formula III′:

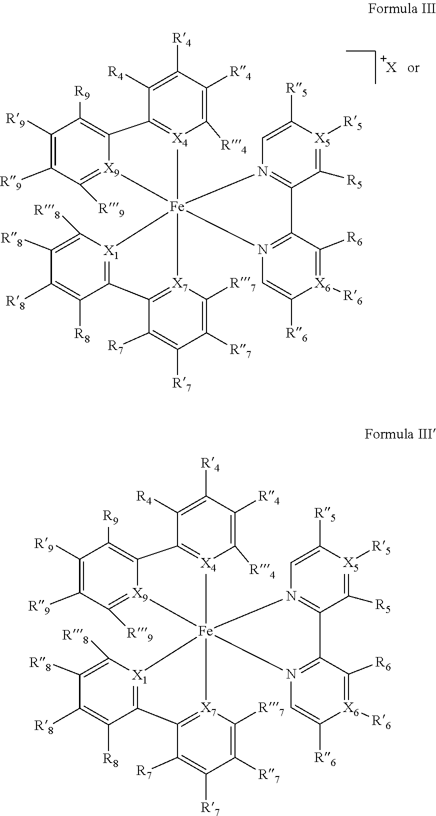

wherein: (i) X₁, X₄, X₅, X₆, X₇, and X₉ can be independently carbon or nitrogen, X can be an anion (e.g., PF₆⁻, Cl⁻, OTf⁻, etc.); (ii) R₄, R′₄, R″₄, R′″₄, R₅, R′₅, R″₅, R₆, R′₆, R″₆, R₇, R′₇, R″₇, R′″₇, R₈, R′₈, R″₈, R′″₈, R₉, R′₉, R″₉, and R′″₉ can be independently hydrogen, substituted or unsubstituted alkyl, substituted or unsubstituted aryl, substituted or unsubstituted heteroaryl, substituted or unsubstituted heterocyclic, or halide; and (iii) the substituents, when present, are independently unsubstituted alkyl, unsubstituted aryl, unsubstituted heteroaryl, unsubstituted polyaryl, unsubstituted heteropolyaryl, unsubstituted alkylaryl, unsubstituted cyclic (e.g., cycloalkyl, cycloalkenyl, cycloalkynyl), unsubstituted heterocyclic, halide, amino, amido, thiol, hydroxyl, cyano, nitro, carbonyl, or alkoxyl.

In some forms, for any of the formulae described herein, X₁, X₅, and X₆ can be carbon, and X₄, X₇, and X₉ can be nitrogen. In some forms, for any of the formulae described herein, X₁, X₉, X₅, and X₆ can be carbon, and X₄ and X₇ can be nitrogen. In some forms, for any of the formulae described herein, X₄, X₇, and X₅ can be carbon, and X₁, X₉, and X₆ can be nitrogen.

In some forms, for any of the formulae described herein, R₁, R′₁, R₂, R′₂, R₃, and R′₃, when present, can be independently hydrogen, unsubstituted alkyl, or unsubstituted aryl. In some forms, for any of the formulae described herein, R₄, R′₄, R₅, R′₅, R₆, R′₆, R₇, R′₇, R₈, R′₈, R₉, and R′₉ can be independently hydrogen, unsubstituted alkyl, unsubstituted aryl, unsubstituted heteroaryl, or halide. In some forms, for any of the formulae described herein, R″₄, R″″₄, R″₅, R″₆, R″₇, R′″₇, R″₈, R′″₈, R′″₉, and R′″₉, when present, can be independently hydrogen, unsubstituted heteroaryl, or halide.

In some forms, the iron (II) complex can have the structure of Formula IV:

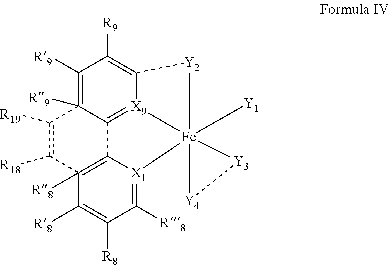

wherein (i) X₁ and X₉ can be independently carbon or nitrogen; (ii) Y₁, Y₂, Y₃, and Y₄ can be independently phosphorus, halogen, substituted or unsubstituted aryl, or substituted or unsubstituted heteroaryl; (iii) R₈, R′₈, R″₈, R′″₈, R₉, R′₉, R″₉, R₁₈ (when present), and R₁₉ (when present) can be independently hydrogen, substituted or unsubstituted alkyl, substituted or unsubstituted aryl, substituted or unsubstituted heteroaryl, substituted or unsubstituted heterocyclic, or halide; and (iv) the substituents, when present, can be independently unsubstituted alkyl, unsubstituted aryl, unsubstituted heteroaryl, unsubstituted polyaryl, unsubstituted heteropolyaryl, unsubstituted alkylaryl, unsubstituted cyclic (e.g., cycloalkyl, cycloalkenyl, cycloalkynyl), unsubstituted heterocyclic, halide, amino, amido, thiol, hydroxyl, cyano, nitro, carbonyl, or alkoxyl.

In some forms, the iron (II) complex can have the structure of Formula V:

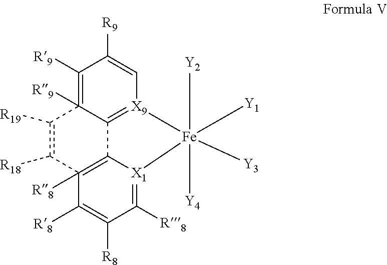

wherein (i) X₁ and X₉ can be independently carbon or nitrogen; (ii) Y₁, Y₂, Y₃, and Y₄ can be independently phosphorus or halogen; (iii) R₈, R′₈, R″₈, R′″₈, R₉, R′₉, R′″₉, R₁₈ (when present), and R₁₉ (when present) can be independently hydrogen, substituted or unsubstituted alkyl, or halide; and (iv) the substituents, when present, can be independently unsubstituted alkyl, unsubstituted aryl, unsubstituted heteroaryl, unsubstituted polyaryl, unsubstituted heteropolyaryl, unsubstituted alkylaryl, unsubstituted cyclic (e.g., cycloalkyl, cycloalkenyl, cycloalkynyl), unsubstituted heterocyclic, halide, amino, amido, thiol, hydroxyl, cyano, nitro, carbonyl, or alkoxyl.

In some forms, X₁ can be nitrogen and X₉ can be carbon. In some forms, Y₁, Y₂, Y₃, and Y₄ can be independently trialkylphosphine (e.g., trimethylphosphine) or halogen. In some forms, R₈, R′₈, R″₈, R′″₈, R₉, R′₉, and R′″₉ can be independently hydrogen or unsubstituted alkyl. R″₈ and R″₉ can be independently hydrogen. In some forms, R₁₈ and R₁₉, when present, can be independently hydrogen or unsubstituted alkyl.

In some forms, the iron (II) complex can have the structure of Formula VI:

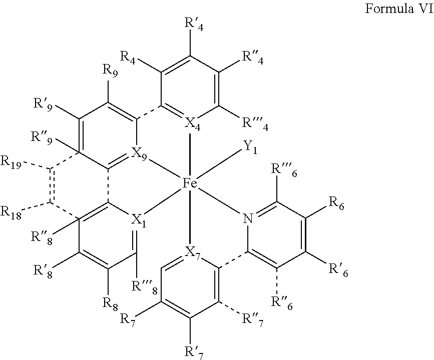

wherein (i) X₁, X₄, X₇, and X₉ can be independently carbon or nitrogen; (ii) Y₁ can be phosphorus, halogen, substituted or unsubstituted aryl, or substituted or unsubstituted heteroaryl; (iii) R₄, R′₄, R″₄, R′″₄, R₆, R′₆, R″₆, R′″₆, R₇, R′₇, R″₇, R₈, R′₈, R″₈, R′″₈, R₉, R′₉, R″₉, R₁₈ (when present), and R₁₉ (when present) can be independently hydrogen, substituted or unsubstituted alkyl, or halide; and (iv) the substituents, when present, can be independently unsubstituted alkyl, unsubstituted aryl, unsubstituted heteroaryl, unsubstituted polyaryl, unsubstituted heteropolyaryl, unsubstituted alkylaryl, unsubstituted cyclic (e.g., cycloalkyl, cycloalkenyl, cycloalkynyl), unsubstituted heterocyclic, halide, amino, amido, thiol, hydroxyl, cyano, nitro, carbonyl, or alkoxyl.

In some forms, the iron (II) complex can have the structure of Formula VII:

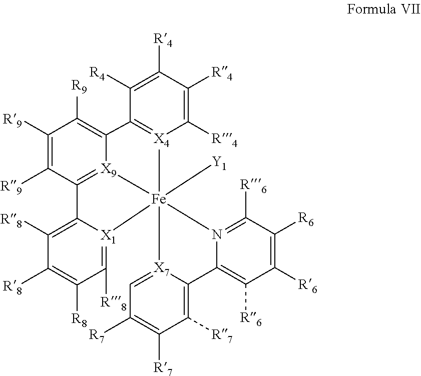

wherein (i) X₁, X₄, X₇, and X₉ can be independently carbon or nitrogen; (ii) Y₁ can be phosphorus or halogen; (iii) R₄, R′₄, R″₄, R′″₄, R₆, R′₆, R″₆, R′″₆, R₇, R′₇, R″₇, R₈, R′₈, R″₈, R′″₈, R₉, R′₉, and R″₉ can be independently hydrogen, substituted or unsubstituted alkyl, or halide; and (iv) the substituents, when present, can be independently unsubstituted alkyl, unsubstituted aryl, unsubstituted heteroaryl, unsubstituted polyaryl, unsubstituted heteropolyaryl, unsubstituted alkylaryl, unsubstituted cyclic (e.g., cycloalkyl, cycloalkenyl, cycloalkynyl), unsubstituted heterocyclic, halide, amino, amido, thiol, hydroxyl, cyano, nitro, carbonyl, or alkoxyl.

In some forms, X₁, X₄, and X₉ can be nitrogen and X₇ can be carbon. In some forms, Y₁ can be a halogen (e.g., fluorine, chlorine, or bromine). In some forms, R₄, R′₄, R″₄, R″₄, R₆, R′₆, R″₆, R′″₆, R₇, R′₇, R″₇, R₈, R′₈, R″₈, R′″₈, R₉, R′₉, and R″₉ can be independently hydrogen or unsubstituted alkyl. In some forms, R″₈ and R″₉ can be independently hydrogen. In some forms, R₁₈ and R₁₉, when present, can be independently hydrogen or unsubstituted alkyl.

For any of the formulae described herein, the alkyl (when present) can be a linear alkyl, a branched alkyl, or a cyclic alkyl (either monocyclic or polycyclic). The terms “cyclic alkyl” and “cycloalkyl” are used interchangeably herein. Exemplary alkyl include a linear C₁-C₃₀ alkyl, a branched C₄-C₃₀ alkyl, a cyclic C₃-C₃₀ alkyl, a linear C₁-C₂₀ alkyl, a branched C₄-C₂₀ alkyl, a cyclic C₃-C₂₀ alkyl, a linear C₁-C₁₀ alkyl, a branched C₄-C₁₀ alkyl, a cyclic C₃-C₁₀ alkyl, a linear C₁-C₆ alkyl, a branched C₄-C₆ alkyl, a cyclic C₃-C₆ alkyl, a linear C₁-C₄ alkyl, cyclic C₃-C₄ alkyl, such as a linear C₁-C₁₀, C₁-C₉, C₁-C₈, C₁-C₇, C₁-C₆, C₁-C₅, C₁-C₄, C₁-C₃, or C₁-C₂ alkyl group, a branched C₃-C₉, C₃-C₉, C₃-C₈, C₃-C₇, C₃-C₆, C₃-C₅, or C₃-C₄ alkyl group, or a cyclic C₃-C₉, C₃-C₉, C₃-C₈, C₃-C₇, C₃-C₆, C₃-C₅, or C₃-C₄ alkyl group. The cyclic alkyl can be a monocyclic or polycyclic alkyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄-C₇, C₄-C₆, or C₄-C₅ monocyclic or polycyclic alkyl group.

For any of the formulae described herein, the alkenyl (when present) can be a linear alkenyl, a branched alkenyl, or a cyclic alkenyl (either monocyclic or polycyclic). The terms “cyclic alkenyl” and “cycloalkenyl” are used interchangeably herein. Exemplary alkenyl include a linear C₂-C₃₀ alkenyl, a branched C₄-C₃₀ alkenyl, a cyclic C₃-C₃₀ alkenyl, a linear C₂-C₂₀ alkenyl, a branched C₄-C₂₀ alkenyl, a cyclic C₃-C₂₀ alkenyl, a linear C₂-C₁₀ alkenyl, a branched C₄-C₁₀ alkenyl, a cyclic C₃-C₁₀ alkenyl, a linear C₂-C₆ alkenyl, a branched C₄-C₆ alkenyl, a cyclic C₃-C₆ alkenyl, a linear C₂-C₄ alkenyl, cyclic C₃-C₄ alkenyl, such as a linear C₂-C₁₀, C₂-C₉, C₂-C₈, C₂-C₇, C₂-C₆, C₂-C₅, C₂-C₄, C₂-C₃ alkenyl group, a branched C₃-C₉, C₃-C₉, C₃-C₈, C₃-C₇, C₃-C₆, C₃-C₅, C₃-C₄ alkenyl group, or a cyclic C₃-C₉, C₃-C₉, C₃-C₈, C₃-C₇, C₃-C₆, C₃-C₅, C₃-C₄ alkenyl group. The cyclic alkenyl can be a monocyclic or polycyclic alkenyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄-C₇, C₄- C₆, or C₄-C₅ monocyclic or polycyclic alkenyl group.

For any of the formulae described herein, the alkynyl (when present) can be a linear alkynyl, a branched alkynyl, or a cyclic alkynyl (either monocyclic or polycyclic). The terms “cyclic alkynyl” and “cycloalkynyl” are used interchangeably herein. Exemplary alkynyl include a linear C₂-C₃₀ alkynyl, a branched C₄-C₃₀ alkynyl, a cyclic C₃-C₃₀ alkynyl, a linear C₂-C₂₀ alkynyl, a branched C₄-C₂₀ alkynyl, a cyclic C₃-C₂₀ alkynyl, a linear C₂-C₁₀ alkynyl, a branched C₄-C₁₀ alkynyl, a cyclic C₃-C₁₀ alkynyl, a linear C₂-C₆ alkynyl, a branched C₄-C₆ alkynyl, a cyclic C₃-C₆ alkynyl, a linear C₁-C₄ alkynyl, cyclic C₃-C₄ alkynyl, such as a linear C₂-C₁₀, C₂-C₉, C₂-C₈, C₂-C₇, C₂-C₆, C₂-C₅, C₂-C₄, C₂-C₃ alkynyl group, a branched C₃-C₉, C₃-C₉, C₃-C₈, C₃-C₇, C₃-C₆, C₃-C₅, C₃-C₄ alkynyl group, or a cyclic C₃-C₉, C₃-C₉, C₃-C₈, C₃-C₇, C₃-C₆, C₃-C₅, C₃-C₄ alkynyl group. The cyclic alkynyl can be a monocyclic or polycyclic alkynyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄- C₇, C₄-C₆, or C₄-C₅ monocyclic or polycyclic alkynyl group.

For any of the formulae described herein, the aryl (when present) can be a C₄-C₃₀ aryl, a C₄-C₂₀ aryl, a C₄-C₁₂ aryl, a C₄-C₁ aryl, a C₄-C₉ aryl, a C₅-C₃₀ aryl, a C₅-C₂₀ aryl, a C₅-C₁₂ aryl, a C₅-C₁₁ aryl, a C₅-C₉ aryl, a C₆-C₂₀ aryl, a C₆-C₁₂ aryl, a C₆-C₁ aryl, or a C₆-C₉ aryl. It is understood that the aryl can be a heteroaryl, such as a C₄-C₃₀ heteroaryl, a C₄-C₂₀ heteroaryl, a C₄-C₁₂ heteroaryl, a C₄-C₁₁ heteroaryl, a C₄-C₉ heteroaryl, a C₅-C₃₀ heteroaryl, a C₅-C₂₀ heteroaryl, a C₅-C₁₂ heteroaryl, a Cₛ-Cₙ heteroaryl, a C₅-C₉ heteroaryl, a C₆-C₃₀ heteroaryl, a C₆-C₂₀ heteroaryl, a C₆-C₁₂ heteroaryl, a C₆-C₁₁ heteroaryl, or a C₆-C₉ heteroaryl. For any of the formulae described herein, the polyaryl group can be a C₈-C₃₀ polyaryl, a C₈-C₂₀ polyaryl, a C₈-C₁₂ polyaryl, a C₈-C₁ polyaryl, a C₁₀-C₃₀ polyaryl, a C₁₀-C₂₀ polyaryl, a C₁₀-C₁₂ polyaryl, a C₁₀-C₁₁ polyaryl, or a C₁₂-C₂₀ polyaryl. It is understood that the aryl can be a polyheteroaryl, such as a C₁₀-C₃₀ polyheteroaryl, a C₁₀-C₂₀ polyheteroaryl, a C₁₀-C₁₂ polyheteroaryl, a C₁₀-C₁₁ polyheteroaryl, or a C₁₂-C₂₀ polyheteroaryl.

Exemplary iron (II) complex are presented below.

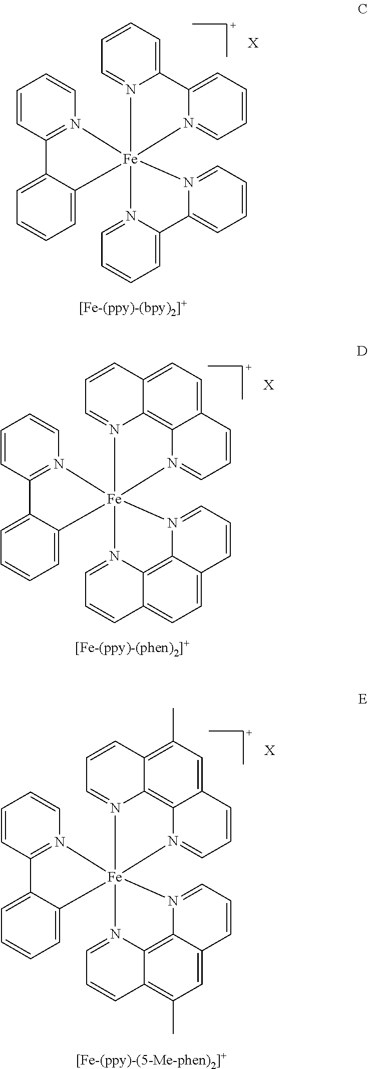

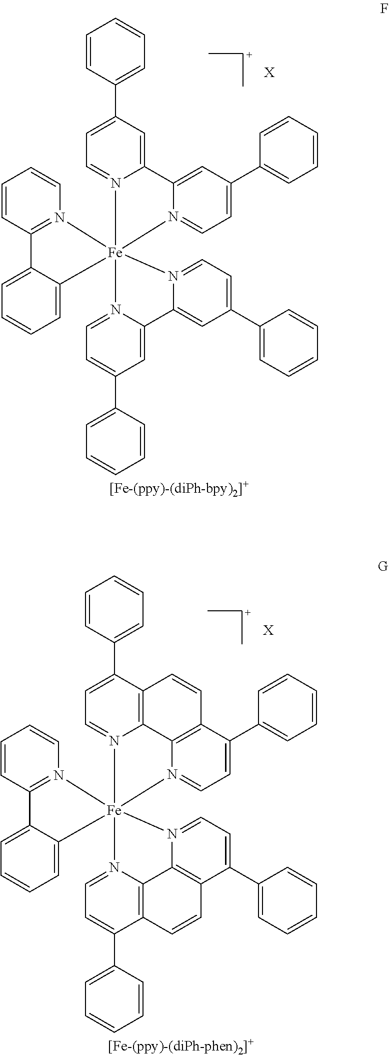

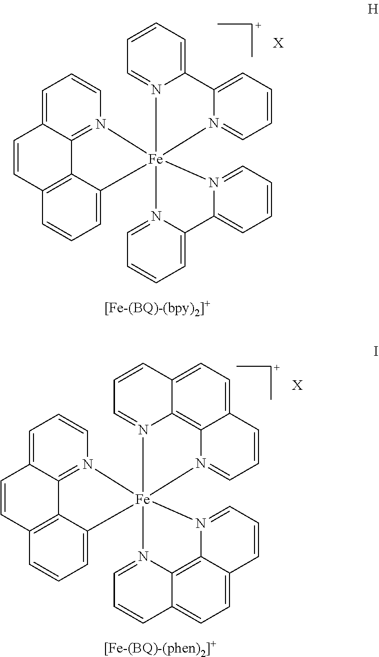

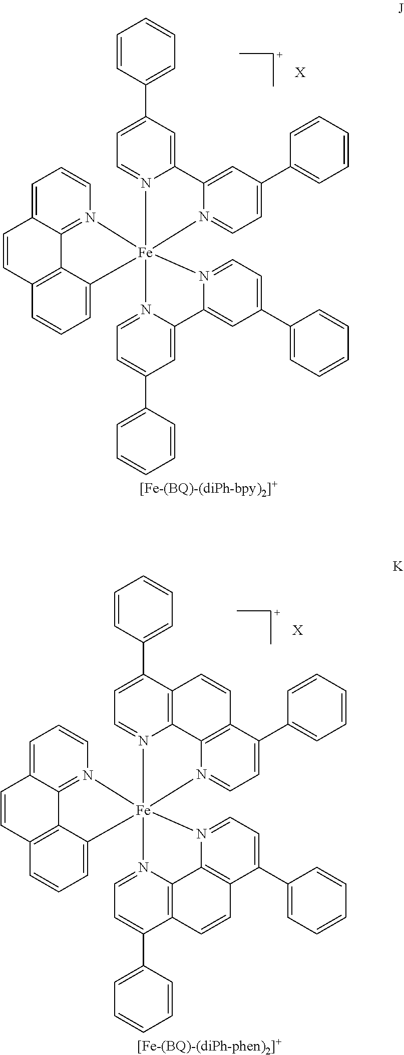

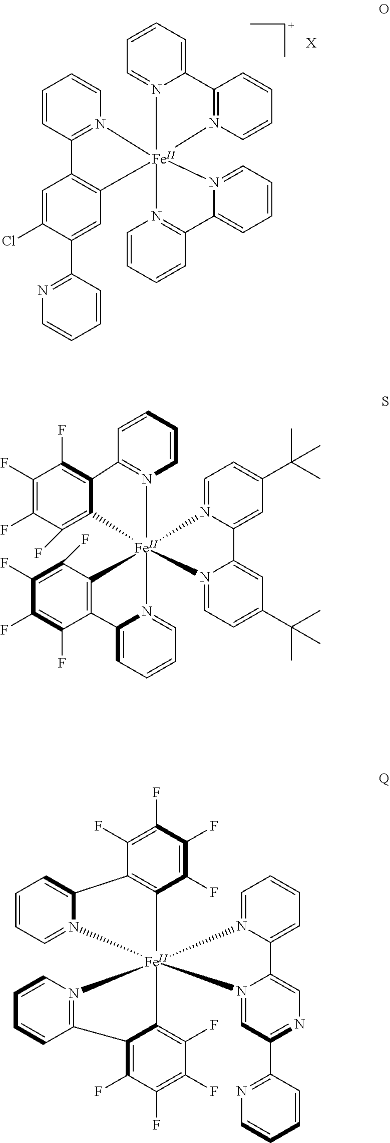

Additional exemplary iron (II) complex are presented below.

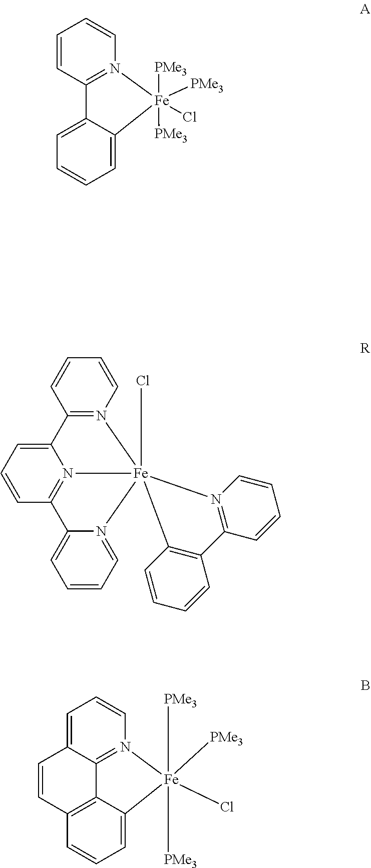

The photophysical properties of the iron (II) complexes disclosed herein can be evaluated by absorption wavelength and/or lifetime. Techniques for measuring the absorption wavelength and lifetime of the iron (II) complexes are known. For example, the absorption wavelength of the iron (II) complexes can be directly measured from the absorption spectra. For example, the lifetime of the iron (II) complexes can be measured using fs-TA spectroscopy in combination with UV-vis spectroelectrochemistry. fs-TA spectroscopy can detect the UV-vis absorptions of transiently lived excited states (ESA) of an iron complex upon photoexcitation, with lifetimes down to a few hundred femtoseconds, and monitor their evolution along the time. Analysis of the kinetics and absorption features of the ESAs, in combination with UV-vis spectroelectrochemistry (where absorptions of electrochemically generated redox derivatives are obtained), can elucidate the electronic structure of the excited states.

In some forms, the iron (II) complexes disclosed herein can have an absorption at a wavelength of at least 600 nm, in a range from 600 nm to about 1100 nm, from 600 nm to about 1000 nm, from 600 nm to about 850 nm, from about 650 nm to about 1100 nm, from about 650 nm to about 1000 nm, from about 650 nm to about 850 nm, from about 700 nm to about 1100 nm, from about 700 nm to about 1000 nm, or from about 700 nm to about 850 nm, such as about 800 nm, obtained based on the absorption spectra of the iron (II) complexes as described above. For example, the iron (II) complexes of Formulae I, I′, II, II′, III, and III′ can have an absorption at a wavelength ranging from 600 nm to about 850 nm or from about 700 nm to about 850 nm. For example, the iron (II) complexes of Formula IV can have an absorption at a wavelength ranging from 600 nm to about 1100 nm or from about 700 nm to about 1100 nm. For example, the iron (II) complexes of Formula V can have an absorption at a wavelength ranging from 600 nm to about 850 nm or from about 700 nm to about 850 nm. For example, the iron (II) complexes of Formula VI or VII (such as complex R) can have an absorption at a wavelength ranging from 600 nm to about 1100 nm or from about 700 nm to about 1100 nm.

In some forms, the iron (II) complexes disclosed herein can have a ³MLCT excited state lifetime of at least 1 ps, at least 2 ps, or at least 2.5 ps, such as about 2.59 ps, obtained based on fs-TA spectroscopy in combination with UV-vis spectroelectrochemistry. In some forms, the lifetime of the disclosed complexes is at least 2 orders higher than those of traditional polypyridine Fe(II) complexes, such as about 2 orders higher than those of traditional polypyridine Fe(II) complexes. The long ³MLCT excited-state lifetime (e.g., approaching ns regime) of the disclosed Fe(II) complexes is desirable for photocatalytic applications involving excited-state electron- or energy-transfer processes. Further, such a lifetime does not exhibit appreciable photoluminescence quantum yields, which is desirable as luminescence does not act as a dominant excited-state decaying pathway that limits the photothermal quantum efficiency. Without being bound to any theories, it is believed that the cyclometallation structure of the disclosed complexes can lead to a strong-field effect. Such a strong ligand field usually directly correlate with longer ³MLCT lifetimes relative to those of traditional polypyridine Fe(II) complexes.

### B. Nanoparticles

The visible-NIR absorbing iron (II) complexes disclosed herein can self-assemble to form nanoparticles (i.e., “Fe nanoparticles” or “Fe NPs”), typically via non-covalent interactions between the iron (II) complexes. The Fe nanoparticles disclosed herein combine the flexibility of metal supramolecular self-assembly and the advantage of near-infrared (NIR) absorbance with high photo-heat conversion efficiency of at least 30% (such as about 60.6%, measured using a 808 nm laser irradiation at 1.0 W/cm for about 20 mins, such as 22 mins), as well as superior photothermal stability under NIR laser irradiation (such as 808 nm) laser irradiation (e.g., decrease of photo-heat conversion efficiency is less than 10% for at least 4 cycles of laser irradiation-cooling, where each laser irradiation lasts at least 5 mins). Photo-heat conversion efficiency of the Fe NPs can be calculated using known method, such as the method described in Roper, D. K., et al., The Journal of Physical Chemistry C, 2007, 111 (9), 3636-3641. For example, the photo-heat conversion efficiency (n) of the Fe NPs can be calculated using the following equation:

$$η = \frac{hS ( {T}_{max} - {T}_{surr} ) - {Q}_{Dis}}{I ( 1 - {10}^{- {A}_{808}} )} \qquad ( 1 )$$

where h (mW/(m²·° C.)) is heat transfer coefficient, S (m²) is the surface area of the container, and Tₘₐₓ is the equilibrium temperature, Tₛᵤᵣ is ambient temperature of the surroundings. Tₘₐₓ−Tₛᵤᵣ can be determined based on a temperature-time graph for the photothermal effect of the Fe NPs, see, e.g., FIG. 13C described in the Examples below. For example, the Tₘₐₓ−Tₛᵤᵣ was determined as 51.3° C. according to FIG. 13C. Q_{Dis} (mW) expresses the heat from light absorbed by a cuvette walls (such as 8.80 mW in the Examples), which can be measured using a quartz cuvette cell containing an aqueous without the Fe NPs. I is the incident laser power (1 W) and A808 is the absorbance (0.579) of supra-CNDs at 808 nm.

To obtain hS, θ is introduced using the maximum system temperature, Tₘₐₓ:

$$θ = \frac{T - {T}_{surr}}{{T}_{max} - {T}_{surr}} \qquad ( 2 )$$

and a sample system time constant τs:

$${τ}_{s} = \frac{{\sum }_{i} {m}_{i} {C}_{p , i}}{hS} \qquad ( 3 )$$

according to the following expression:

$$t = - {τ}_{s} ln ( θ ) \qquad ( 4 )$$

τs can be first determined and thus hS can be deduced. For example, in the Examples below, τs was determined to be 473.13 s thus hS was deduced to be 9.91 mW/° C. (substituted m=1 g, C=4.2 J/g·K in Equation (3)).

Finally, the photo-heat conversion efficiency (η) of Fe NPs can be calculated from equation 1 shown above.

In some forms, the photo-heat conversion efficiency is significantly higher than those that are currently available, for example, gold nanorods: 21.0%, Cu₂-xSe: 22% and Cu₉S₅: 25.7% (Wang, S., et al., Advanced Materials, 2016, 28 (38), 8379-8387; Hessel, C. M., et al., Nano Letters, 2011, 11 (6), 2560-2566; and Tian, Q., et al., ACS Nano, 2011, 5 (12), 9761-9771). Without being bound to any theories, it is believed that the energy level in dd state of the iron (II) complexes forming the particles is low, such that they can transfer more energy from light to heat, and thereby achieve enhanced photothermal conversion efficiency.

The disclosed Fe nanoparticles generally have an average diameter less than 200 nm, allowing them to target tumor by enhanced permeability retention (EPR) effect. For example, the disclosed Fe nanoparticles have an average morphology diameter ranging from about 50 nm to about 150 nm or from about 80 nm to about 100 nm, as measured using transmission electron microscopy in solid form. For example, the disclosed Fe nanoparticles have an average hydrodynamic diameter ranging from about 60 nm to about 200 nm, such as about 90 nm, as measured using dynamic light scattering in an aqueous solvent, such as water.

Further, the disclosed Fe nanoparticles have excellent tumor-targeted photothermal efficacies, and good biosafety in vitro and in vivo, as demonstrated by the data shown in the Examples below. In some forms, the Fe NPs are coated with a coating agent, such as bovine serum albumin (“BSA”). The coating agent may be associated with the Fe NPs via any suitable interactions, such as electrostatic interactions.

Coating with the coating agent can further enhance the tumor accumulations and biocompatibility of the metallosupramolecular particles in vivo, such as shown by the photothermal effect in mice treated with coated Fe NPs, compared to mice treated with Fe NPs without coating, under an 808 nm laser irradiation for a suitable period of time (e.g., about 5 mins, about 6 mins, about 10 mins, about 20 mins, etc.). For example, the coated Fe NPs shows an in vivo photothermal effect (e.g., mice treated with the coated Fe NPs, under an 808 nm laser irradiation for about 5 to about 20 mins) that is at least 1.5-time, at least 2-time, from 1.5-time to 10-time, from 1.5-time to 5-time, or from 1.5-time to 3-time stronger than the in vivo photothermal effect of Fe NPs without coating (e.g., mice treated with the Fe NPs without coating, at the same dose and under the same laser irradiation for the same time period as the mice treated with the coated Fe NPs). See, e.g., FIGS. 13F and 13G described in the Examples below. The excellent photothermal performance of the Fe NPs allow them to solve the problem of low photothermal conversion efficiency associated with most existing photothermal materials (such as those described above).

Other examples of coating agents that are suitable for coating the Fe nanoparticles include, but are not limited to, polyalkylenes or copolymers thereof (e.g., Pluronic® F-127, DSPE-PEG (2000)), and polysaccharides (e.g., hyaluronic acid), and combinations thereof.

In some forms, the disclosed Fe nanoparticles further include a targeting moiety capable of binding a target biological substance, such as target tumor cells, to improve cell uptake, tumor targeting, and/or intratumoral retention time of the nanoparticles. The target moiety may be attached to the surface of the Fe nanoparticles or to the coating of coated Fe nanoparticles. Typically, the targeting moiety are arranged such that they are exposed to a surrounding environment. Exemplary targeting moiety suitable for use in the disclosed nanoparticles include, but are not limited to, an antibody, an antibody fragment, an antibody mimetic, an affibody, a nucleic acid, an oligonucleotide, an aptamer, a peptide, a steroid, histidine tag, NTA-Ni, and a tyrosine kinase inhibitor, and a combination thereof.

In some forms, the disclosed Fe nanoparticles further include an active agent, such as one or more anticancer agents. The active agent may be encapsulated in the Fe nanoparticles or coated Fe nanoparticles, embedded in the coating of the coated Fe nanoparticles, or associated to the surface of the Fe nanoparticles or coated Fe nanoparticles. Examples of anticancer agents that can be used in the nanoparticles include, but are not limited to, temozolomide, carmustine, bevacizumab, procarbazine, lomustine, vincristine, gefitinib, erlotinib, cisplatin, carboplatin, oxaliplatin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vinblastine, vindesine, vinorelbine, paclitaxel, taxol, docetaxel, etoposide, teniposide, amsacrine, topotecan, camptothecin, bortezomib, anagrelide, tamoxifen, toremifene, raloxifene, droloxifene, iodoxyfene, fulvestrant, bicalutamide, flutamide, nilutamide, cyproterone, goserelin, leuprorelin, buserelin, megestrol, anastrozole, letrozole, vorozole, exemestane, finasteride, marimastat, trastuzumab, cetuximab, dasatinib, imatinib, combretastatin, thalidomide, azacitidine, azathioprine, capecitabine, chlorambucil, cyclophosphamide, cytarabine, daunorubicin, doxifluridine, epothilone, irinotecan, mechlorethamine, mercaptopurine, mitoxantrone, pemetrexed, tioguanine, valrubicin and/or lenalidomide or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); and methotrexate, vincristine, doxorubicin, cisplatin (MVAC); and combinations thereof. Additional anticancer agents are described in U.S. Pat. No. 10,393,736, the disclosure of which is incorporated herein by reference in its entirety.

### C. Pharmaceutical Composition Containing Fe NPs or Coated Fe NPs

Pharmaceutical compositions that contain a plurality of the Fe nanoparticles or coated Fe nanoparticles described herein in a form suitable for in vitro or in vivo applications (such as administration to a mammal), are disclosed. The pharmaceutical composition may include one or more pharmaceutically acceptable carriers and/or one or more pharmaceutically acceptable excipients. For example, the pharmaceutical composition may be in the form of a liquid, such as a solution or a suspension, and contain a plurality of the disclosed Fe nanoparticles or coated Fe nanoparticles in an aqueous medium and, optionally, one or more suitable excipients for the liquid composition. Optionally, the pharmaceutical composition is in a solid form, and contains a plurality of the disclosed Fe nanoparticles or coated Fe nanoparticles and one or more suitable excipients for a solid composition.

### 1. Carriers and Excipients

Suitable pharmaceutically acceptable carriers and excipients are generally recognized as safe (GRAS), and may be administered to an individual without causing undesirable biological side effects or unwanted interactions.

Representative carriers and excipients include solvents (including buffers), diluents, pH modifying agents, preservatives, antioxidants, suspending agents, wetting agents, viscosity modifiers, tonicity agents, and stabilizing agents, and a combination thereof.

Excipients can be added to a liquid or solid pharmaceutical composition (for in vivo or in vitro applications) to assist in sterility, stability (e.g. shelf-life), integration, and to adjust and/or maintain pH or isotonicity of the Fe nanoparticles or coated Fe nanoparticles in the pharmaceutical composition, such as diluents, pH modifying agents, preservatives, antioxidants, suspending agents, wetting agents, viscosity modifiers, tonicity agents, and stabilizing agents, and a combination thereof.

Fe nanoparticles or coated Fe nanoparticles for administering to a subject in need thereof, such as a mammal, can be dissolved or suspended in a suitable carrier to form a liquid pharmaceutical formulation, such as sterile saline, phosphate buffered saline (PBS), balanced salt solution (BSS), viscous gel, or other pharmaceutically acceptable carriers for administration. The pharmaceutical formulation may also be a sterile solution, suspension, or emulsion in a nontoxic, parenterally acceptable diluent or solvent.

### 2. Forms for Administration

A plurality of the disclosed Fe nanoparticles or coated Fe nanoparticles can be formulated into a pharmaceutical composition, in a liquid form or a solid form, as a liquid formulation or a solid formulation for oral administration or parenteral administration (e.g. intramuscular administration, intravenous administration, intraperitoneal administration, and subcutaneous administration) to a subject.

a. Oral Compositions

The pharmaceutical composition containing a plurality of the disclosed Fe nanoparticles or coated Fe nanoparticles may be provided in a form suitable for oral administration to a subject, such as a mammal (i.e., an oral composition). Oral administration may involve swallowing, so that the Fe nanoparticles or coated Fe nanoparticles, optionally including active agent(s) in/on the nanoparticles, enter the gastrointestinal tract, or buccal or sublingual administration may be employed by which the Fe nanoparticles or coated Fe nanoparticles enter the blood stream directly from the mouth.

Compositions suitable for oral administration include solid compositions such as tablets, capsules containing particulates, liquids, powders, lozenges (including liquid-filled lozenges), chews, multi- and nano-particulates, gels, solid solutions, liposomes, films, ovules, sprays, and liquid compositions.

Liquid compositions for oral administration include suspensions, solutions, syrups, and elixirs. Such oral compositions may be employed as fillers in soft or hard capsules and can contain one or more suitable carriers and/or excipients, for example, water, ethanol, polyethylene glycol, propylene glycol, chitosan polymers and chitosan derivatives (e.g. N-trimethylene chloride chitosan, chitosan esters, chitosan modified with hydrophilic groups, such as amino groups, carboxyl groups, sulfate groups, etc.), methylcellulose, a suitable oil, one or more emulsifying agents, and/or suspending agents. Liquid compositions for oral administration may also be prepared by the reconstitution of a solid, for example, from a sachet.

Optionally, the Fe nanoparticles or coated Fe nanoparticles are included in a fast-dissolving and/or fast-disintegrating dosage form.

For tablet or capsule dosage forms, in addition to the Fe nanoparticles or coated Fe nanoparticles described herein, tablets generally contain disintegrants, binders, diluents, surface active agents, lubricants, glidants, antioxidants, colourants, flavoring agents, preservatives, or taste masking agents, or a combination thereof.

Examples of suitable disintegrants for forming a tablet or capsule dosage form containing the Fe nanoparticles or coated Fe nanoparticles include, but are not limited to, sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant can have a concentration in a range from about 1 wt % to about 25 wt %, from about 5 wt % to about 20 wt % of the tablet or capsule dosage form containing the Fe NPs or coated Fe NPs.

Binders are generally used to impart cohesive qualities to a tablet composition containing the Fe nanoparticles or coated Fe nanoparticles. Suitable binders for forming a tablet or capsule formulation containing the Fe nanoparticles or coated Fe nanoparticles include, but are not limited to, microcrystalline cellulose, gelatin, sugars, polyethylene glycol, natural and synthetic gums, polyvinylpyrrolidone, pregelatinised starch, chitosan polymers and chitosan derivatives (e.g. N-trimethylene chloride chitosan, chitosan esters, chitosan modified with hydrophilic groups, such as amino groups, carboxyl groups, sulfate groups, etc.), hydroxypropyl cellulose, and hydroxypropyl methylcellulose.

Suitable diluents for forming a tablet or capsule composition containing the Fe nanoparticles or coated Fe nanoparticles include, but are not limited to, lactose (as, for example, the monohydrate, spray-dried monohydrate or anhydrous form), chitosan polymers and chitosan derivatives (e.g. N-trimethylene chloride chitosan, chitosan esters, chitosan modified with hydrophilic groups, such as amino groups, carboxyl groups, sulfate groups, etc.), N-sulfonated derivatives of chitosan, quaternarized derivatives of chitosan, carbosyalkylated chitosan, microcrystalline chitosan, mannitol, xylitol, dextrose, sucrose, sorbitol, microcrystalline cellulose, starch and dibasic calcium phosphate dihydrate.

Tablet or capsule composition containing the Fe nanoparticles or coated Fe nanoparticles may also contain surface active agents, such as sodium lauryl sulfate and polysorbate 80, and glidants such as silicon dioxide and talc, in the coating. When present, surface active agents can have a concentration in a range from about 0.2 wt % to 5 wt % of the tablet or capsule formulation.

Tablet or capsule compositions containing the Fe nanoparticles or coated Fe nanoparticles also generally contain lubricants, such as magnesium stearate, calcium stearate, zinc stearate, sodium stearyl fumarate, and mixtures of magnesium stearate with sodium lauryl sulphate. Lubricants can have a concentration in a range from about 0.25 wt % to 10 wt %, from about 0.5 wt % to about 3 wt % of the tablet or capsule composition.

Other possible excipients included in a tablet or capsule formulation containing the Fe nanoparticles or coated Fe nanoparticles include glidants (e.g. Talc or colloidal anhydrous silica at about 0.1 wt % to about 3 wt % of the tablet or capsule formulation), antioxidants, colourants, flavouring agents, preservatives and taste-masking agents. When present, glidants can have a concentration in a range from about 0.2 wt % to 1 wt % of the tablet or capsule composition.

An exemplary tablet composition contains up to about 80 wt % of the Fe nanoparticles or coated Fe nanoparticles described herein, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet or capsule blends, including the Fe nanoparticles or coated Fe nanoparticles and one or more suitable excipients, may be compressed directly or by roller to form tablets. Tablet or capsule blends or portions of the blends may alternatively be wet-, dry-, or melt-granulated, melt congealed, or extruded before tableting. The final tablet or capsule composition may contain one or more layers and may be coated or uncoated; it may even be encapsulated in a particle, such as a polymeric particle or a liposomal particle.

Solid formulations containing the Fe nanoparticles or coated Fe nanoparticles for oral administration may be formulated to be immediate and/or modified release. Modified release formulations include delayed, sustained, pulsed, controlled, targeted and programmed release formulations.

b. Parenteral Formulations

Optionally, the pharmaceutical composition containing a plurality of the disclosed Fe nanoparticles or coated Fe nanoparticles is in a form suitable for administration directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular, and subcutaneous delivery. Suitable means for parenteral administration include needle (including microneedle) injectors, needle-free injectors, and infusion techniques.

For example, the pharmaceutical formulation containing a plurality of the Fe nanoparticles or coated Fe nanoparticles is in a form suitable for intramuscular administration, intravenous administration, intraperitoneal administration, or subcutaneous administration, or a combination thereof.

Parenteral formulations containing the Fe nanoparticles or coated Fe nanoparticles described herein are typically aqueous solutions which can contain excipients such as salts, carbohydrates and buffering agents (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4), but, for some applications, they may be more suitably formulated as a sterile aqueous solution or as a dried form to be used in conjunction with a suitable vehicle such as sterile, pyrogen-free water.

The liquid compositions containing the Fe nanoparticles or coated Fe nanoparticles for parenteral administration may be a solution, a suspension, or an emulsion.

The liquid pharmaceutically acceptable carrier forming the parenteral composition containing the Fe nanoparticles or coated Fe nanoparticles can include one or more physiologically compatible buffers, such as a phosphate buffers. One skilled in the art can readily determine a suitable saline content and pH for an aqueous carrier for administration (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4).

Liquid compositions containing the Fe nanoparticles or coated Fe nanoparticles for parenteral administration may include one or more suspending agents, such as cellulose derivatives, sodium alginate, polyvinylpyrrolidone, gum tragacanth, or lecithin. The liquid compositions may also include one or more preservatives, such as ethyl or n-propyl p-hydroxybenzoate.

Optionally, the liquid composition containing the Fe nanoparticles or coated Fe nanoparticles contains one or more solvents that are low toxicity organic (i.e., nonaqueous) class 3 residual solvents, such as ethanol, acetone, ethyl acetate, tetrahydofuran, ethyl ether, and propanol, and a combination thereof. Any such solvents included in the liquid formulation should not detrimentally react with the one or more active agents present in the Fe nanoparticles or coated Fe nanoparticles in the liquid composition. Solvents such as freon, alcohol, glycol, polyglycol, or fatty acid, can also be included in the liquid composition containing the Fe nanoparticles or coated Fe nanoparticles as desired to increase the volatility of the solution or suspension.

Liquid compositions containing the Fe nanoparticles or coated Fe nanoparticles for parenteral administration may also contain minor amounts of polymers, surfactants, or other pharmaceutically acceptable excipients known to those in the art. In this context, “minor amounts” means an amount that is sufficiently small to avoid adversely affecting uptake of the Fe nanoparticles and coated Fe nanoparticles by the targeted cells, such as pituitary gonadotrophs.

The preparation of parenteral compositions containing the Fe nanoparticles or coated Fe nanoparticles is typically under sterile conditions, for example, by lyophilisation, which can be accomplished using standard pharmaceutical techniques known to those skilled in the art.

Compositions for parenteral administration containing the Fe nanoparticles or coated Fe nanoparticles may be formulated to provide immediate and/or modified release of the active agent. Modified release formulations include delayed, sustained, pulsed, controlled, targeted and programmed release formulations.

c. Amount of Fe NPs or Coated Fe NPs

Pharmaceutical compositions typically contain an effective amount of the Fe nanoparticles or coated Fe nanoparticles and one or more pharmaceutically acceptable carriers and/or excipients. As used herein, the term “effective amount” means any amount of the Fe nanoparticles or coated Fe nanoparticles that is sufficient to achieve the desired therapeutic, prophylactic, and/or diagnostic effect on a biological sample or in a subject to which it is administered. Depending on the condition to be treated and/or the route of administration, such an effective amount of the Fe nanoparticles or coated Fe nanoparticles can be between 0.01 to 1000 mg per kilogram body weight of the subject per day, between 0.1 and 500 mg, such as between 1 and 250 mg, for example about 5, 10, 20, 50, 100, 150, 200 or 250 mg, per kilogram body weight of the subject per day, which can be administered as a single daily dose, divided over one or more daily doses. The amount of the Fe nanoparticles or coated Fe nanoparticles administered, the route of administration, and the further treatment regimen can be determined by the treating clinician or testing technologies, depending on factors such as the age, gender and general condition of the subject, the nature and severity of the disease/symptoms being prevented, treated, or diagnosed, and/or the samples being tested.

In some forms, the total amount of the Fe nanoparticles or coated Fe nanoparticles in the pharmaceutical composition can be in a range from about 1 μM to about 1 mM, from about 10 μM to about 500 μM, from about 10 μM to about 200 μM, or from about 25 μM to about 100 μM. The term “total concentration of the Fe nanoparticles or coated Fe nanoparticles in the pharmaceutical composition” refers to the sum of the weight of the Fe nanoparticles or coated Fe nanoparticles in the composition relative to the weight of the composition.

In some forms, the pharmaceutical composition is in a unit dosage form, and can be suitably packaged, for example in a box, blister, vial, bottle, sachet, ampoule or in any other suitable single-dose or multi-dose holder or container (which can be properly labeled); optionally with one or more leaflets containing product information and/or instructions for use. Generally, such unit dosages can contain between 1 and 1000 mg, and usually between 5 and 500 mg, of the disclosed Fe nanoparticles or coated Fe nanoparticles, e.g., about 10, 25, 50, 100, 200, 300 or 400 mg per unit dosage.

### III. Methods of Use

The complexes and particles formed therefrom are suitable for use as photothermal transduction agents. In particular, the Fe NPs and coated Fe NPs formed from the complexes have excellent photothermal performance and can target tumor by EPR effect.

For example, the Fe NPs and coated Fe NPs disclosed herein have appreciable near-infrared (NIR) absorbance with high photo-heat conversion efficiency of at least 30% (such as about 60%, measured using a 808 nm laser irradiation at 1.0 W/cm for about 20 mins, such as 22 mins). Such a photo-heat conversion efficiency is significantly higher than commercial gold nanorods (21.0%), Cu₂-xSc (22%), and Cu₉S₅ (25.7%) (Wang, S., et al., Advanced Materials, 2016, 28 (38), 8379-8387; Hessel, C. M., et al., Nano Letters, 2011, 11 (6), 2560-2566; and Tian, Q., et al., ACS Nano, 2011, 5 (12), 9761-9771). Additionally or alternatively, the disclosed Fe NPs and coated Fe NPs have superior photothermal stability under near-infrared (e.g., 808 nm) laser irradiation (e.g., decrease of photo-heat conversion efficiency is less than 10% for at least 4 cycles of laser irradiation-cooling, where each laser irradiation lasts at least 5 mins). Further, the disclosed Fe nanoparticles generally have an average diameter less than 200 nm, allowing them to target tumor by EPR effect.

In some forms, the disclosed Fe NPs, coated NPs, or pharmaceutical composition containing the Fe NPs or coated NPs can be used for treating a cancer. Preferably, the cancer is a solid cancer (also referred to herein as a “tumor”). “Treatment” or “treating” refers to the medical management of a patient with the intent to cure, ameliorate, stabilize, or prevent a disease, pathological condition, or disorder, such as a cancer. This term includes active treatment, that is, treatment directed specifically toward the improvement of a disease, pathological condition, or disorder, such as a cancer; and also includes causal treatment, that is, treatment directed toward removal of the cause of the associated disease, pathological condition, or disorder, such as a cancer. In addition, this term includes palliative treatment, that is, treatment designed for the relief of symptoms rather than the curing of the disease, pathological condition, or disorder, such as a cancer; preventative treatment, that is, treatment directed to minimizing or partially or completely inhibiting the development of the associated disease, pathological condition, or disorder, such as a cancer; and supportive treatment, that is, treatment employed to supplement another specific therapy directed toward the improvement of the associated disease, pathological condition, or disorder, such as a cancer.

In some forms, a method for treating cancer, such as a solid cancer, using a pharmaceutical composition containing the Fe NPs or coated NPs includes: (i) administering the pharmaceutical composition to a subject in need thereof; and (ii) applying a laser irradiation to the subject or a target region of the subject.

The administration in step (i) can be performed using any suitable technique, such as oral administration, intramuscular administration, intravenous administration, intraperitoneal administration, or subcutaneous administration, or a combination thereof.

Typically, in step (ii), the laser irradiation has a wavelength of at least 670 nm, in a range from 670 nm to 1500 nm, from 670 nm to 1200 nm, from 670 nm to 1000 nm, from 700 nm to 1500 nm, from 700 nm to 1200 nm, or from 700 nm to 1000 nm, such as about 800 nm, for example 808 nm. The laser irradiation can be operated under any suitable laser power density, such as a laser power density ranging from about 0.01 W/cm² to about 10 W/cm², from about 0.05 W/cm² to about 10 W/cm², from about 0.1 W/cm² to about 10 W/cm², from about 0.1 W/cm² to about 5 W/cm², from about 0.01 W/cm² to about 1 W/cm², from about 0.01 W/cm² to about 0.5 W/cm², from about 0.05 W/cm² to about 1 W/cm², from about 0.05 W/cm² to about 0.5 W/cm², from about 0.1 W/cm² to about 1 W/cm², from about 0.1 W/cm² to about 0.5 W/cm², from about 0.01 W/cm² to about 0.3 W/cm², from about 0.05 W/cm² to about 0.3 W/cm², or from about 0.1 W/cm² to about 0.3 W/cm², for example, about 1 W/cm², about 0.5 W/cm², or about 0.3 W/cm². In some forms, the laser irradiation is operated under a laser power density such that a mild PTT is obtained. In these forms, the laser power density under which the laser irradiation operated is, for example, less than 0.3 W/cm², such as from about 0.01 W/cm² to about 0.3 W/cm², from about 0.05 W/cm² to about 0.3 W/cm², or from about 0.1 W/cm² to about 0.3 W/cm², e.g., about 0.3 W/cm².

In step (ii), the laser irradiation is maintained for a time period sufficient to achieve a desired treatment effect, for example, a reduction in tumor volume and/or tumor weight. Generally, the laser irradiation is maintained for a time period ranging from about 1 min to about 1 hour, from about 5 mins to about 1 hour, from about 1 min to about 30 mins, from about 5 mins to about 30 mins, from about 1 min to about 20 mins, from about 5 mins to about 20 mins, from about 1 min to about 15 mins, or from about 5 mins to about 15 mins, such as about 6 mins.

Step (i) (administration of pharmaceutical composition) or step (ii) (applying a laser irradiation), or steps (i) and (ii) of the disclosed method may occur one or more times. For example, the administration step is performed one or more times, and following all of the administration steps, a laser irradiation is applied to the subject or a target region of the subject once. For example, the administration step is performed one or more times, and following all of the administration steps, a laser irradiation is applied to the subject or a target region of the subject two or more times. For example, the administration is performed once, and the laser irradiation is applied to the subject one or more times. For example, the administration step is performed once, and then a laser irradiation is applied to the subject or a target region of the subject two or more times. For example, the administration step is performed one or more times, and following all of the administration steps, a laser irradiation is applied to the subject or a target region of the subject, which can be performed one or more times. For example, the administration step is performed once, and then a laser irradiation is applied to the subject or a target region of the subject once, following which another administration step and a subsequent laser irradiation step are performed to repeat the cycle.

When more than one administration step or more than one laser irradiation step, or more than one cycle of administration and laser irradiation are performed, each of the steps or cycles can be performed regularly every 5 mins, every 10 mins, every 20 mins, every 30 mins, every hour, every 2 hours, every day, every two days, every 3 days, every week, every two weeks, every month, etc.; or irregularly with a time interval of 5 mins, 10 mins, 20 mins, 30 mins, 1 hour, 2 hours, 1 day, 2 days, 3 days, 5 days, 1 week, 2 weeks, etc. For example, the administration step is performed once, and the laser irradiation step is performed more than two times on day 1, day 3, day 5, etc., following the administration of the pharmaceutical composition or all of the administrations of the pharmaceutical composition (when step (i) is performed more than one time). For example, the administration step is performed once, and the laser irradiation step is performed two times on day 1 and day 3 following the administration of the pharmaceutical composition or all of the administrations of the pharmaceutical composition (when step (i) is performed more than one time). For example, a first cycle of administration and laser irradiation is performed on day 1, a second cycle of administration and laser irradiation is performed on day 3, and a third cycle of administration and laser irradiation is performed on day 5. The specific number of treatment and time interval for the treatment can be determined by the treating clinician or testing technologies, depending on factors such as the age, gender and general condition of the subject, the nature and severity of the disease/symptoms being prevented, treated, or diagnosed, and/or the samples being tested.

Step (ii) (applying a laser irradiation) can be performed immediately (such as within 2 hours, within 1 hour, or within 30 mins) following step (i) (administration of pharmaceutical composition) or performed at a later time following step (i), such as performed 24 hours, 48 hours, 72 hours, 5 days, 7 days, 2 weeks, 1 month, and/or 3 months following administration or all of the administrations of the pharmaceutical composition (when step (i) is performed more than one time). For example, a laser irradiation is applied to the subject or a target region of the subject on day 1, on day 2, on day 3, on day 4, and/or on day 5 post administration of the pharmaceutical composition or all of the administrations of the pharmaceutical composition (when step (i) is performed more than one time). For example, a laser irradiation is applied to the subject or a target region of the subject on day 1 and day 3 post administration of the pharmaceutical composition or all of the administrations of the pharmaceutical composition (when step (i) is performed more than one time).

Typically, following the administration step or all of the administration steps, an effective amount of the Fe NPs or coated Fe NPs in the pharmaceutical formulation is administered to the subject, such that following step (ii) applying a laser radiation, cancer cells are killed and/or growth or proliferation of the cancer cells are reduced or prevented, and thereby ameliorate one or more symptoms associated with the cancer in the subject, such as reduce the tumor volume and/or tumor weight. Optionally, the cancer treatment effect occurs without any significant side effects, such as death of normal cells.

For example, following the administration step or all of the administration steps, the dosage of the metallosupramolecular particles administered to the subject is from about 0.1 μg to about 100 μg, from about 0.5 μg to about 50 μg, from about 1 μg to about 100 μg, from about 1 μg to about 50 μg, from about 1 μg to about 25 μg, from about 1 μg to about 10 μg, from about 1 μg to about 5 μg, from about 4 μg to about 10 μg, or from about 1 μg to about 4 μg per g of the subject. Following step (ii) or all of step (ii) (if step (ii) occurs more than one time), the tumor volume is reduced by at least 10%, at least 20%, at least 30%, at least 40%, or at least 50%, compared to the tumor volume before administration of the pharmaceutical composition; and/or the tumor weight is reduced by at least 50%, at least 60%, at least 70%, or at least 80% compared to the tumor weight before administration of the pharmaceutical composition, optionally without any cytotoxicity to normal cells as indicated by standard hematology markers and/or blood biochemical parameters compared to a control administered with the pharmaceutically acceptable excipient(s) only.

The cancer treatment effect, such as tumor volume and/or tumor weight reduction, can occur immediately following the treatment or in a few days following the treatment. For example, the volume and/or weight reduction of the tumor occurs in about 1 day, two days, three days, five days, one week, two weeks, three weeks, or one month after step (ii) or all of step (ii).

In some forms, the disclosed method can further include administering an active agent to the subject before step (i) administering a pharmaceutical composition containing the Fe nanoparticles or coated Fe nanoparticles, simultaneously with step (i), after step (i) and prior to step (ii) applying laser radiation, or after step (ii), or a combination thereof. The active agent can be any agents that assist the photothermal treatment effect of the Fe nanoparticles or coated Fe nanoparticles. In some forms, the active agent can be an anticancer agent, such as any of those described above. In some forms, the active agent can be an immunotherapeutic agent, such as immunoregulators for target molecules on immune cells (e.g., activators of CD4 and/or CD8) and immunoregulators for tumor antigens release and immune-stimulatory molecules activation by ablated tumor cells (e.g., oligodeoxynucleotides containing cytosine-guanine (CpG) motifs, imiquimod (R837), anti-PD-L1 antibodies (aPD-L1), CpG, Anti-CTL A4 Ab, Anti-PD-L1 Ab, Adoptive T cell therapy, etc.). Additional exemplary immunoregulators that can be used in combination with PTT are described in Nam, et al. Nature Reviews Materials 4.6 (2019): 398-414.

In some forms, the disclosed method can further include a step of forming the pharmaceutical composition containing Fe NPs or coated Fe NPs, prior to step (i) or in step (i). In some forms, the pharmaceutical composition is formed by adding one or more of the iron (II) complex(es) disclosed herein in an aqueous solvent to form the pharmaceutical composition containing Fe NPs. In some forms, the pharmaceutical composition is formed by (a) dissolving one or more of the iron (II) complex disclosed herein in a first solvent, such as an organic solvent (e.g., DMSO), to form an iron (II) complex solution, (b) adding the iron (II) complex solution into an aqueous solvent to form Fe NPs, and optionally (c) collecting the Fe NPs and optionally washing the Fe NPs. The formed Fe NPs can be then added in a carrier to form the pharmaceutical composition containing Fe NPs. When coated Fe NPs are used in the pharmaceutical composition, an additional coating step is performed after formation of the Fe NPs in the aqueous solvent. For example, after collecting and washing the Fe NPs formed in an aqueous solvent, the Fe NPs and a coating agent are added in a suitable solvent, optionally under sonication, at room temperature, for a suitable period of time to form the coated Fe NPs. Specific exemplary conditions for forming Fe NPs and coated Fe NPs are described in the Examples below.

The disclosed Fe(II) complexes, metallosupramolecular particles, and methods can be further understood through the following enumerated paragraphs.

1. An iron (II) complex having a structure of:

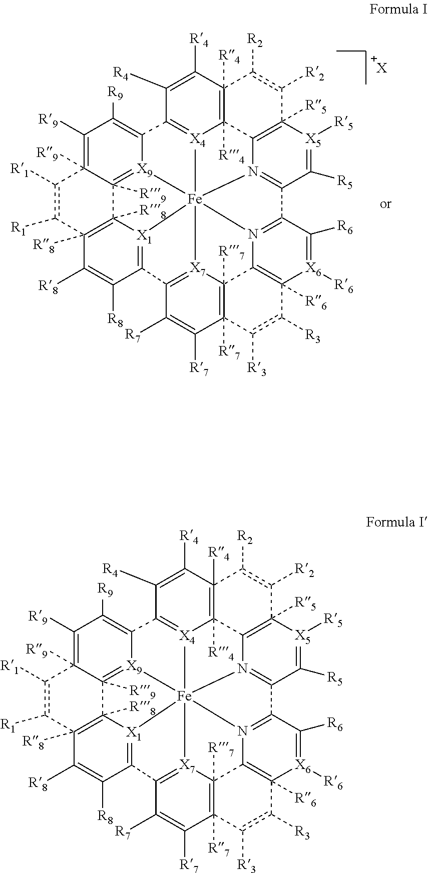

wherein:
(i) X₁, X₄, X₅, X₆, X₇, and X₉ are independently carbon or nitrogen, X is an anion;
(ii) each [symbol] is absent or a single bond, each [symbol] is absent or a double bond;
(iii) R₁, R′₁, R₂, R′₂, R₃, and R′₃, when present, are independently hydrogen, substituted or unsubstituted alkyl, substituted or unsubstituted aryl, or substituted or unsubstituted alkylaryl;
(iv) R₄, R′₄, R₅, R′₅, R₆, R′₆, R₇, R′₇, R₈, R′₈, R₉, and R′₉, are independently hydrogen, substituted or unsubstituted alkyl, substituted or unsubstituted aryl, substituted or unsubstituted heteroaryl, substituted or unsubstituted polyaryl, substituted or unsubstituted heteropolyaryl, substituted or unsubstituted alkylaryl, substituted or unsubstituted cyclic, substituted or unsubstituted heterocyclic, halide, amino, amido, thiol, hydroxyl, cyano, nitro, carbonyl, or alkoxyl;
(v) R″₄, R′″₄, R″₅, R″₆, R″₇, R′″₇, R″₈, R′″₈, R″₉, and R′″₉, when present, are independently hydrogen, halide, substituted or unsubstituted alkyl, substituted or unsubstituted aryl, substituted or unsubstituted heteroaryl, substituted or unsubstituted cyclic, or substituted or unsubstituted heterocyclic; and
(vi) the substituents, when present, are independently unsubstituted alkyl, unsubstituted aryl, unsubstituted heteroaryl, unsubstituted polyaryl, unsubstituted heteropolyaryl, unsubstituted alkylaryl, unsubstituted cyclic, unsubstituted heterocyclic, halide, amino, amido, thiol, hydroxyl, cyano, nitro, carbonyl, or alkoxyl.
   2. The iron (II) complex of paragraph 1, having a structure of:

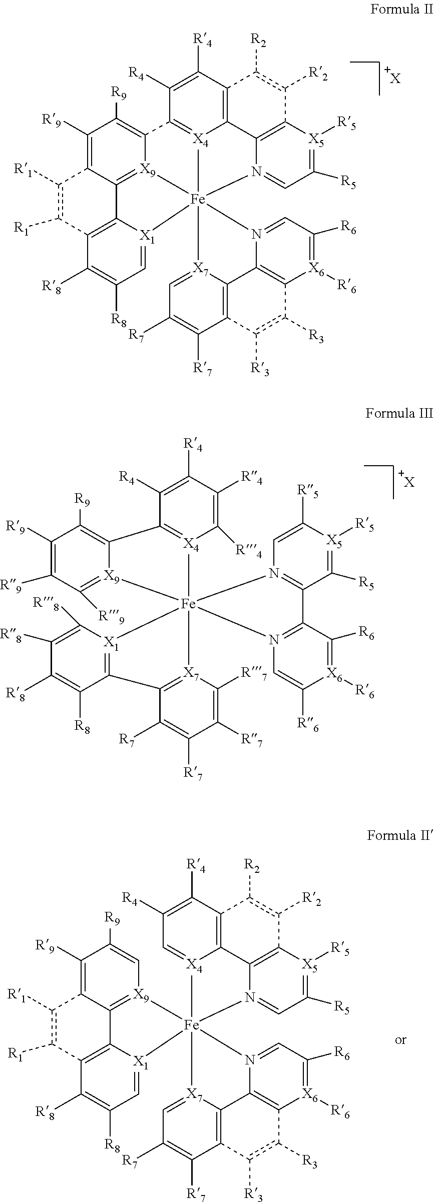

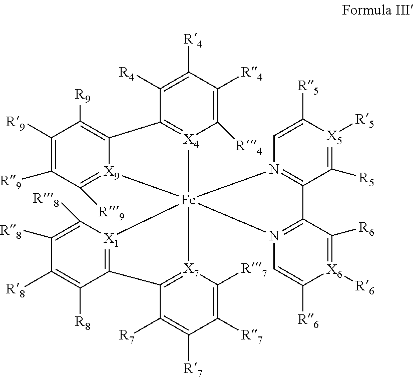

wherein:
(i) X₁, X₄, X₅, X₆, X₇, and X₉ are independently carbon or nitrogen, X is an anion;
(ii) each [symbol] is absent or a single bond, each [symbol] is absent or a double bond;
(iii) R₁, R′₁, R₂, R′₂, R₃, and R′₃, when present, are independently hydrogen, substituted or unsubstituted alkyl, or substituted or unsubstituted aryl;
(iv) R₄, R′₄, R″₄, R′″₄, R₅, R′₅, R″₅, R₆, R′₆, R″₆, R₇, R′₇, R′″₇, R′″₇, R₈, R′₈, R″₈, R′″₈, R₉, R′₉, R′″₉, and R′″₉ are independently hydrogen, substituted or unsubstituted alkyl, substituted or unsubstituted aryl, substituted or unsubstituted heteroaryl, substituted or unsubstituted heterocyclic, or halide; and
(v) the substituents, when present, are independently unsubstituted alkyl, unsubstituted aryl, unsubstituted heteroaryl, unsubstituted polyaryl, unsubstituted heteropolyaryl, unsubstituted alkylaryl, unsubstituted cyclic, unsubstituted heterocyclic, halide, amino, amido, thiol, hydroxyl, cyano, nitro, carbonyl, or alkoxyl.
   3. The iron (II) complex of paragraph 1 or 2, wherein:
(i) X₁, X₅, and X₆ are carbon, and X₄, X₇, and X₉ are nitrogen; or
(ii) X₁, X₉, X₅, and X₆ are carbon, and X₄ and X₇ are nitrogen; or
(iii) X₄, X₇, and X₅ are carbon, and X₁, X₉, and X₆ are nitrogen.
   4. The iron (II) complex of any one of paragraphs 1-3, wherein:
(i) R₁, R′₁, R₂, R′₂, R₃, and R′₃, when present, are independently hydrogen, unsubstituted alkyl, or unsubstituted aryl;
(ii) R₄, R′₄, R₅, R′₅, R₆, R′₆, R₇, R′₇, R₈, R′₈, R₉, and R′₉ are independently hydrogen, unsubstituted alkyl, unsubstituted aryl, unsubstituted heteroaryl, or halide; and
(iii) R″₄, R′″₄, R″₅, R″₆, R″₇, R′″₇, R″₈, R′″₈, R′″₉, and R′″₉, when present, are independently hydrogen, unsubstituted heteroaryl, or halide.
   5. The iron (II) complex of any one of paragraphs 1-4, having a structure of:

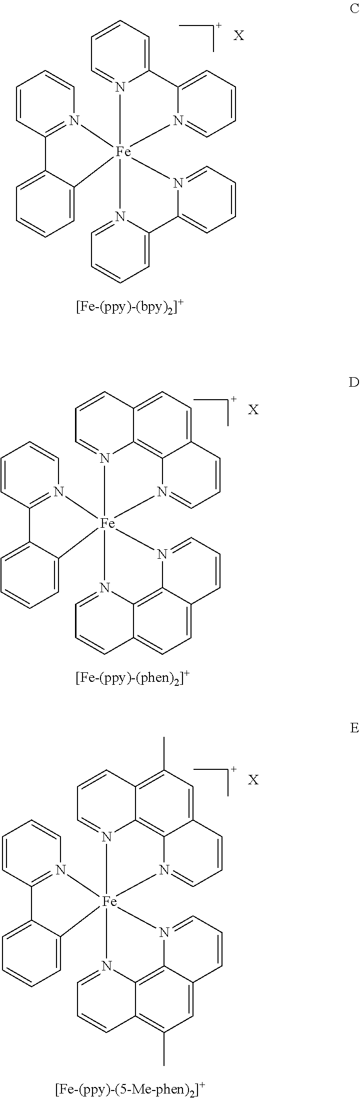

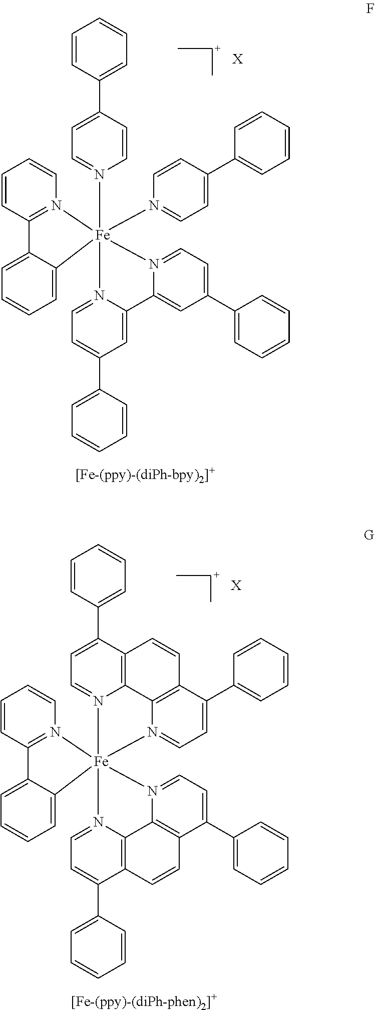

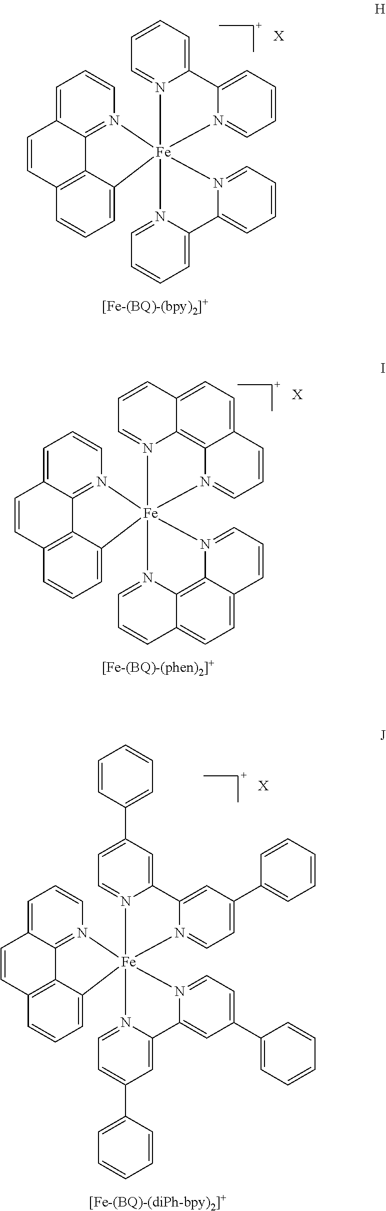

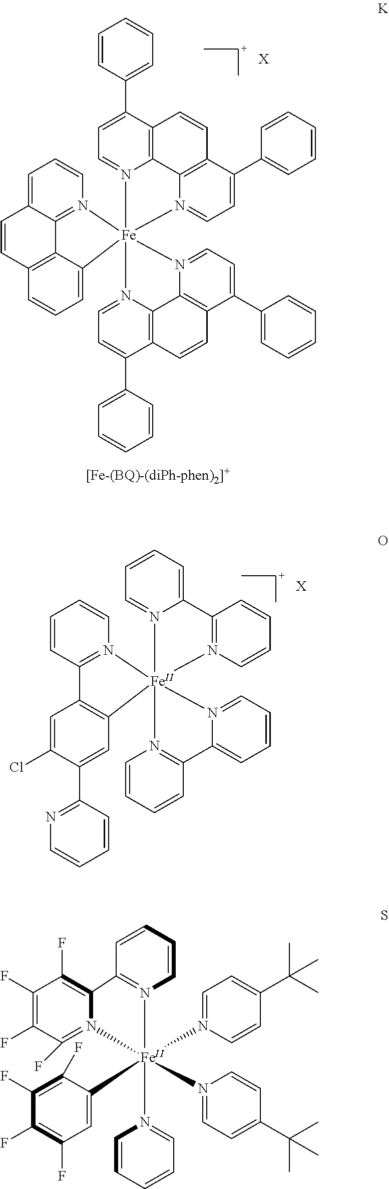

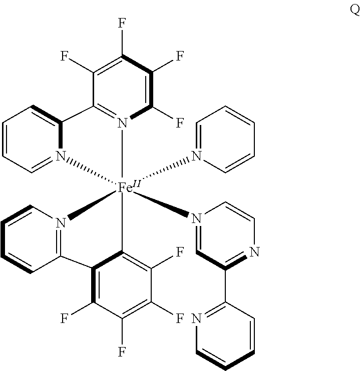

6. The iron (II) complex of any one of paragraphs 1-5, having an absorption at a wavelength of at least 650 nm, such as ranging from 600 nm to about 850 nm or from about 700 nm to about 850 nm, and/or a lifetime of at least 1 ps.
7. Metallosupramolecular particles comprising the iron (II) complex of any one of paragraphs 1-6.
8. The metallosupramolecular particles of paragraph 7, further comprising a coating agent, wherein the coating agent is covalently or non-covalently attached to the surface of the metallosupramolecular particles, and optionally wherein the coating agent is bovine serum albumin, polyalkylene or a copolymer thereof (e.g., Pluronic® F-127, DSPE-PEG (2000)), or a polysaccaride (e.g., hyaluronic acid), or a combination thereof.
9. The metallosupramolecular particles of paragraph 7 or 8, further comprising a targeting moiety.
10. The metallosupramolecular particles of any one of paragraphs 7-9, having an average morphology diameter ranging from about 50 nm to about 150 nm or from about 80 nm to about 100 nm, as measured using transmission electron microscopy; and/or an average hydrodynamic diameter ranging from about 60 nm to about 200 nm, such as about 90 nm, as measured using dynamic light scattering.
11. The metallosupramolecular particles of any one of paragraphs 7-10, having a photo-heat conversion efficiency of at least 30%, at least 40%, at least 50%, or at least 60%, measured using a 808 nm laser irradiation at 1.0 W/cm for about 20 min.
12. The metallosupramolecular particles of any one of paragraphs 7-11, having a photothermal stability for at least four laser irradiation-cooling cycles, each laser irradiation lasts at least 5 mins.
13. A pharmaceutical composition comprising the metallosupramolecular particles of any one of paragraphs 7-12, and optionally one or more pharmaceutically acceptable excipients.
14. The pharmaceutical composition of paragraph 13, wherein the metallosupramolecular particles are in an amount ranging from about 1 μM to about 1 mM, from about 10 μM to about 500 μM, from about 10 μM to about 200 μM, or from about 25 μM to about 100 μM.
15. A method for treating cancer using the pharmaceutical composition of paragraph 13 or 14 comprising:
(i) administering the pharmaceutical composition to a subject in need thereof; and
(ii) applying a laser irradiation to the subject or a target region of the subject, wherein step (i), step (ii), or steps (i) and (ii) occurs one or more times.
   16. The method of paragraph 15, wherein in step (ii) the laser irradiation has a wavelength of at least 670 nm, in a range from 670 nm to 1500 nm, from 670 nm to 1200 nm, from 670 nm to 1000 nm, from 700 nm to 1500 nm, from 700 nm to 1200 nm, or from 700 nm to 1000 nm, such as about 800 nm; has a laser power density ranging from about 0.01 W/cm² to about 10 W/cm², from about 0.05 W/cm² to about 10 W/cm², from about 0.1 W/cm² to about 10 W/cm², from about 0.1 W/cm² to about 5 W/cm², from about 0.01 W/cm² to about 1 W/cm², from about 0.01 W/cm² to about 0.5 W/cm², from about 0.05 W/cm² to about 1 W/cm², from about 0.05 W/cm² to about 0.5 W/cm², from about 0.1 W/cm² to about 1 W/cm², from about 0.1 W/cm² to about 0.5 W/cm², from about 0.01 W/cm² to about 0.3 W/cm², from about 0.05 W/cm² to about 0.3 W/cm², or from about 0.1 W/cm² to about 0.3 W/cm², for example, about 1 W/cm², about 0.5 W/cm², or about 0.3 W/cm²; and/or is maintained for a time period ranging from about 1 min to about 1 hour, from about 5 mins to about 1 hour, from about 1 min to about 30 mins, from about 5 mins to about 30 mins, from about 1 min to about 20 mins, from about 5 mins to about 20 mins, from about 1 min to about 15 mins, or from about 5 mins to about 15 mins.
   17. The method of paragraph 15 or 16, wherein the pharmaceutical composition is administered by oral administration, intramuscular administration, intravenous administration, intraperitoneal administration, or subcutaneous administration, or a combination thereof.
   18. The method of any one of paragraphs 15-17, further comprising administering an active agent, optionally wherein the active agent is an anticancer agent.
   19. The method of any one of paragraphs 15-18, wherein in step (i), the dosage of the metallosupramolecular particles administered is from about 0.1 μg to about 100 μg, from about 0.5 μg to about 50 μg, from about 1 μg to about 100 μg, from about 1 μg to about 50 μg, from about 1 μg to about 25 μg, from about 1 μg to about 10 μg, from about 1 μg to about 5 μg, from about 4 μg to about 10 μg, or from about 1 μg to about 4 μg per g of the subject.
   20. The method of any one of paragraphs 15-19, wherein following step (ii) or all of step (ii) (if step (ii) occurs more than one time), the tumor volume is reduced by at least 10%, at least 20%, at least 30%, at least 40%, or at least 50%, compared to the tumor volume before administration of the pharmaceutical composition; and/or the tumor weight is reduced by at least 50%, at least 60%, at least 70%, or at least 80% compared to the tumor weight before administration of the pharmaceutical composition, optionally without any cytotoxicity to normal cells as indicated by standard hematology markers and/or blood biochemical parameters compared to a control administered with the pharmaceutically acceptable excipient(s) only.
   21. An iron (II) complex having a structure of:

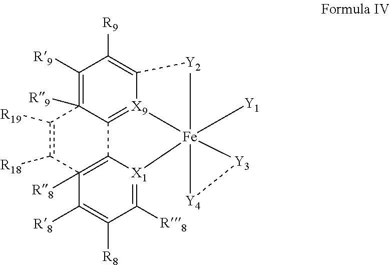

wherein (i) X₁ and X₉ are independently carbon or nitrogen; (ii) Y₁, Y₂, Y₃, and Y₄ are independently phosphorus, halogen, substituted or unsubstituted aryl, or substituted or unsubstituted heteroaryl; (iii) R₈, R′₈, R″₈, R′″₈, R₉, R′₉, R′″₉, R₁₈ (when present), and R₁₉ (when present) are independently hydrogen, substituted or unsubstituted alkyl, substituted or unsubstituted aryl, substituted or unsubstituted heteroaryl, substituted or unsubstituted heterocyclic, or halide; and (iv) the substituents, when present, are independently unsubstituted alkyl, unsubstituted aryl, unsubstituted heteroaryl, unsubstituted polyaryl, unsubstituted heteropolyaryl, unsubstituted alkylaryl, unsubstituted cyclic (e.g., cycloalkyl, cycloalkenyl, cycloalkynyl), unsubstituted heterocyclic, halide, amino, amido, thiol, hydroxyl, cyano, nitro, carbonyl, or alkoxyl.
   22. The iron (II) complex of paragraph 21, having a structure of:

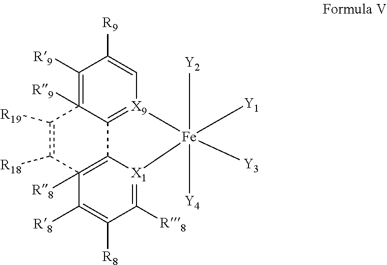

wherein (i) X₁ and X₉ are independently carbon or nitrogen; (ii) Y₁, Y₂, Y₃, and Y₄ are independently phosphorus or halogen; (iii) R₈, R′₈, R″₈, R′″₈, R₉, R′₉, R″₉, R₁₈ (when present), and R₁₉ (when present) are independently hydrogen, substituted or unsubstituted alkyl, or halide; and (iv) the substituents, when present, are independently unsubstituted alkyl, unsubstituted aryl, unsubstituted heteroaryl, unsubstituted polyaryl, unsubstituted heteropolyaryl, unsubstituted alkylaryl, unsubstituted cyclic (e.g., cycloalkyl, cycloalkenyl, cycloalkynyl), unsubstituted heterocyclic, halide, amino, amido, thiol, hydroxyl, cyano, nitro, carbonyl, or alkoxyl.
   23. The iron (II) complex of paragraph 22, wherein:
X₁ is nitrogen and X₉ is carbon;
Y₁, Y₂, Y₃, and Y₄ are independently trialkylphosphine (e.g., trimethylphosphine) or halogen; or
R₈, R′₈, R″₈, R′″₈, R₉, R′₉, and R″₉ are independently hydrogen or unsubstituted alkyl; or
R″₈ and R″₉ are independently hydrogen; or
R₁₈ and R₁₉, when present, are independently hydrogen or unsubstituted alkyl; or a combination thereof.
   24 The iron (II) complex of paragraph 21, having a structure of:

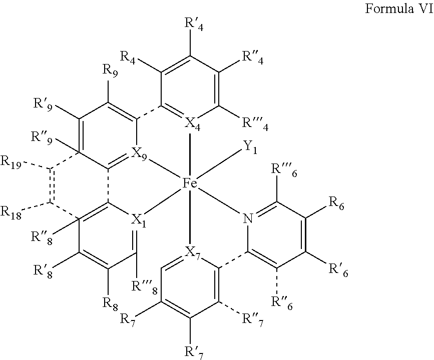

wherein (i) X₁, X₄, X₇, and X₉ are independently carbon or nitrogen; (ii) Y₁ is phosphorus, halogen, substituted or unsubstituted aryl, or substituted or unsubstituted heteroaryl; (iii) R₄, R′₄, R″₄, R′″₄, R₆, R′₆, R″₆, R′″₆, R₇, R′₇, R″₇, R₈, R′₈, R″₈, R′″₈, R₉, R′₉, R″₉, R₁₈ (when present), and R₁₉ (when present) are independently hydrogen, substituted or unsubstituted alkyl, or halide; and (iv) the substituents, when present, are independently unsubstituted alkyl, unsubstituted aryl, unsubstituted heteroaryl, unsubstituted polyaryl, unsubstituted heteropolyaryl, unsubstituted alkylaryl, unsubstituted cyclic (e.g., cycloalkyl, cycloalkenyl, cycloalkynyl), unsubstituted heterocyclic, halide, amino, amido, thiol, hydroxyl, cyano, nitro, carbonyl, or alkoxyl.
   25. The iron (II) complex of paragraph 24, having a structure of:

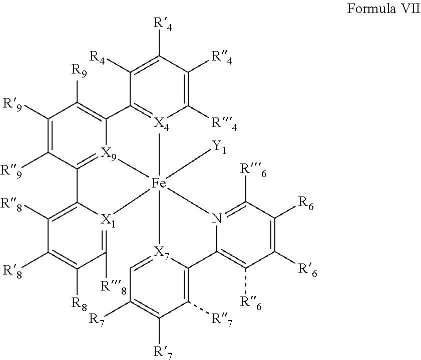

wherein (i) X₁, X₄, X₇, and X₉ are independently carbon or nitrogen; (ii) Y₁ is phosphorus or halogen; (iii) R₄, R′₄, R″₄, R′″₄, R₆, R′₆, R″₆, R′″₆, R₇, R′₇, R″₇, R₈, R′₈, R″₈, R′″₈, R₉, R′₉, and R″₉ are independently hydrogen, substituted or unsubstituted alkyl, or halide; and (iv) the substituents, when present, are independently unsubstituted alkyl, unsubstituted aryl, unsubstituted heteroaryl, unsubstituted polyaryl, unsubstituted heteropolyaryl, unsubstituted alkylaryl, unsubstituted cyclic (e.g., cycloalkyl, cycloalkenyl, cycloalkynyl), unsubstituted heterocyclic, halide, amino, amido, thiol, hydroxyl, cyano, nitro, carbonyl, or alkoxyl.
   26. The iron (II) complex of paragraph 24 or 25, wherein:
X₁, X₄, and X₉ are nitrogen and X₇ is carbon;
Y₁ is halogen (e.g., fluorine, chlorine, or bromine); or
R₄, R′₄, R″₄, R′″₄, R₆, R′₆, R″₆, R′″₆, R₇, R′₇, R″₇, R₈, R′₈, R″₈, R′″₈, R₉, R′₉, and R″₉ are independently hydrogen or unsubstituted alkyl;
R″₈ and R″₉ are independently hydrogen; or
R₁₈ and R₁₉, when present, are independently hydrogen or unsubstituted alkyl; or a combination thereof.

The present invention will be further understood by reference to the following non-limiting examples.

27. The iron (II) complex of any one of paragraphs 21-26, having a structure of:

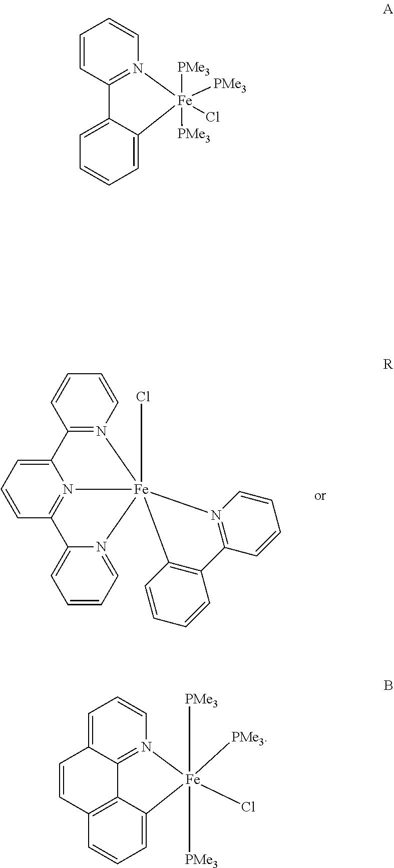

### EXAMPLES

### Example 1. Synthesis and Spectroscopic Studies of Exemplary Cyclometalated Fe(II) Complexes

A series of air-stable monocyclometalated tris(bidentate) [Fe^{II}(ppy)(N\hat{ }N)₂]⁺ and [Fe^{II}(BQ)(N\hat{ }N)₂]⁺ (ppy=2-(pyridin-2-yl)benzen-1-ide, BQ=benzo[h]quinolin-10-ide) complexes have been synthesized and characterized. The electronic absorption and electrochemical properties of the complexes have been studied and compared with the benchmark [Fe^{I}(bpy)₃]²⁺ complex, revealing a profound σ-donating effect of the cyclometalating donor. Three biscyclometalated tris(bidentate) Fe complexes and an [Fe^{II}(ppy)(tpy)Cl]complex (tpy=2,2′:6′,2″-terpyridine) were also synthesized and characterized.

Compared to that of [Fe(bpy)₃]²⁺, notable bathochromic shift by ˜170-180 nm and more intense absorptions in the visible region were observed for the exemplary iron (II) complexes. Electrochemical studies showed drastic cathodic shifts of the E_{1/2}(FeIII/II) by ˜0.95 V (e.g., complexes C—O) or ˜1.05 V (e.g., complex R), and a notable HOMO-LUMO narrowing relative to those of [Fe(bpy)₃]²⁺. The UV-vis and CV results demonstrate a profound σ-donating ability of the cyclometalating ligand in the Fe(II) complexes, and a stronger donating effect in a tris(bidentate) than in a bis (tridentate) fashion. A lifetime of 2.59 ps ascribable to a ³MLCT excited state of complex G was revealed by fs-TA measurements, which indicate the beneficial effect of the cyclometallation structure in enhancing the ³MLCT lifetimes of Fe(II) complexes. Further, complex R is a useful precursor for the synthesis of cyclometalated Fe derivatives, where the Cl could be replaced by a series of substituents for catalytic or photochemical studies. Further, complex R showed low-energy absorption with λₘₐₓ=998, 805 nm and extending to 1100 nm.

### Materials and Methods

### Synthesis of Exemplary Mononuclear Complexes

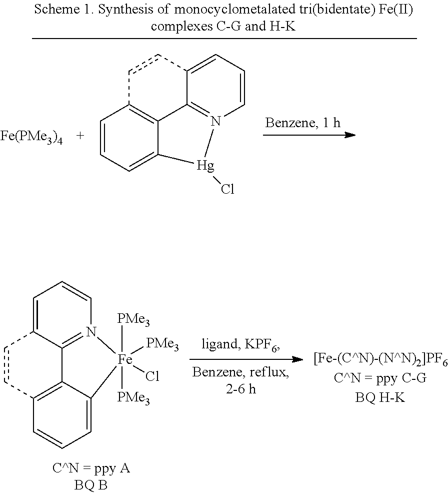

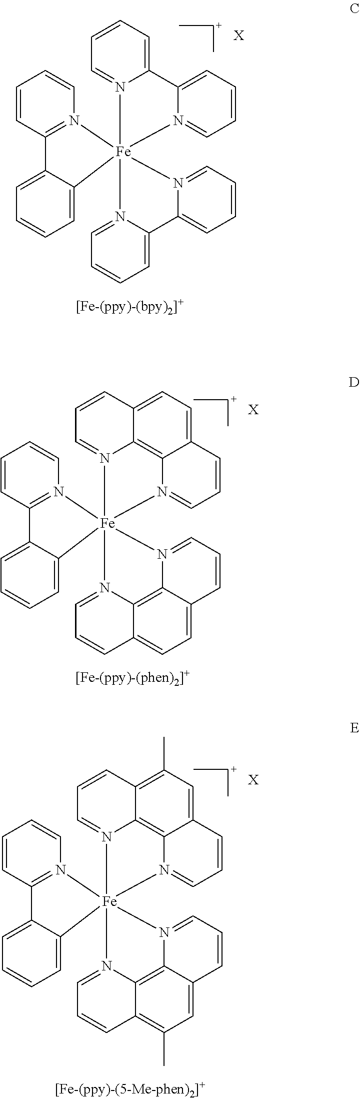

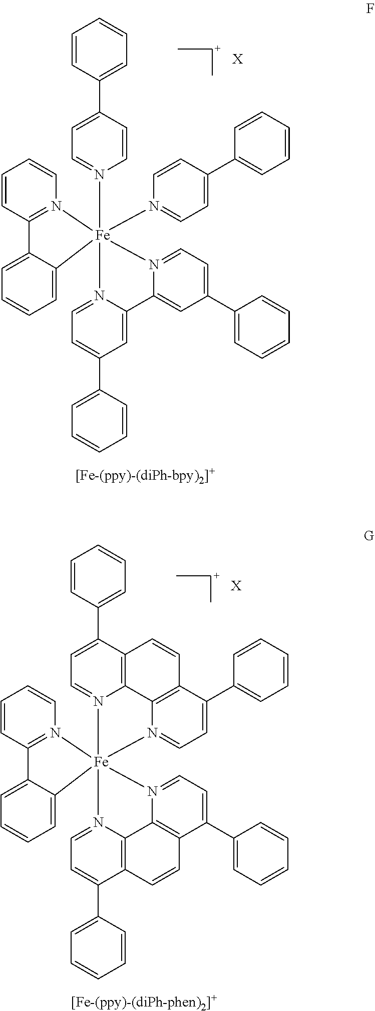

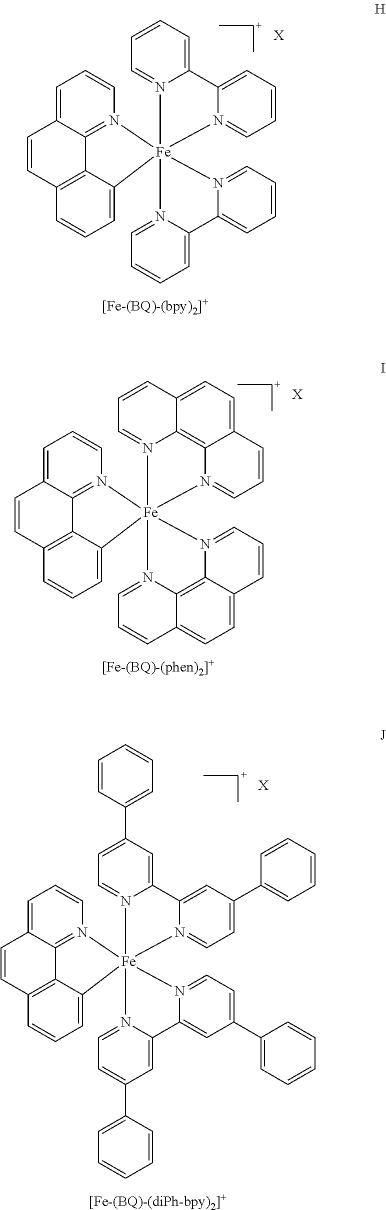

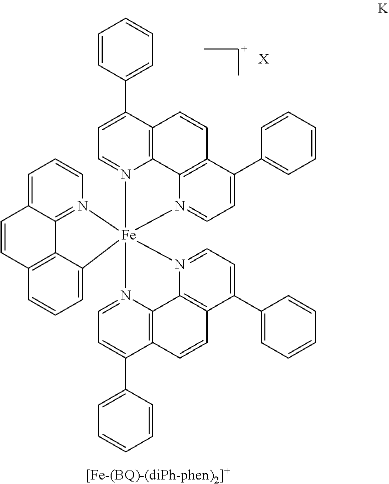

Reaction of Fe(PMe₃)₄ with Hg-(ppy)-Cl or Hg-(BG)-Cl in benzene gave a dark brownish red species A, or a deep purple red species B, respectively, in half an hour (Scheme 1). Both A and B were air-sensitive in solution and not isolated. Reaction of A and B with the bidentate diamine ligands gave complexes C-K. A triplet at 22.6 ppm and a doublet at 14.7 ppm (J=56.8 Hz) with a ratio of ˜1:2 for A, and a triplet at 26.0 ppm and a doublet at 14.4 ppm (J=56.9 Hz) for B, respectively, were observed in their ³¹P NMR spectra (data not shown). Complexes A and F were thus postulated to be Fe-(C\hat{ }N) ppy/BQ-(PMe₃)₃-Cl. This assignment was supported by the ESI-MS measurements (data not shown). Refluxing the mixture of the benzene filtrate of the Fe-(C\hat{ }N)-(PMe₃)₃-Cl compounds and the N\hat{ }N ligand in the presence of KPF₆ afforded light-colored solutions with dark precipitates in 2-6 hours. After solvent evaporation, the dark solids were washed with benzene or ether and subjected to column purification over neutral alumina, affording the complexes in relatively pure forms in 60-82% yield with E obtained as two stereoisomers in 1:1.1 ratio. The bpy complexes C and H were deep-green in color; the bpy complex J and the phen complexes D, E, I, G and K were deep-blue; and complex F was dark brown.

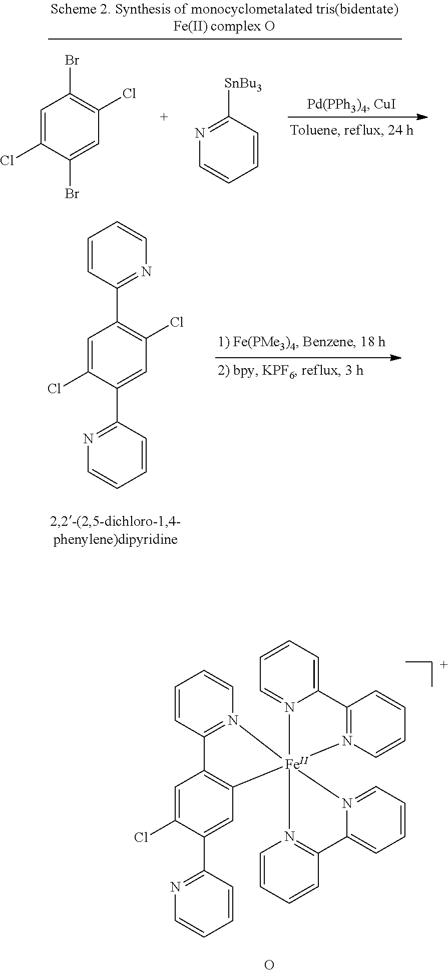

Mononuclear complex O arising from a single oxidative addition process using Fe(PMe₃)₄ and 2,2′-(2,5-dichloro-1,4-phenylene)dipyridine as the precursors was isolated as the major product (Scheme 2), with the second Cl atom on the 2,2′-(2,5-dichloro-1,4-phenylene)dipyridine ligand precursor intact as reveal by HR ESI-MS.

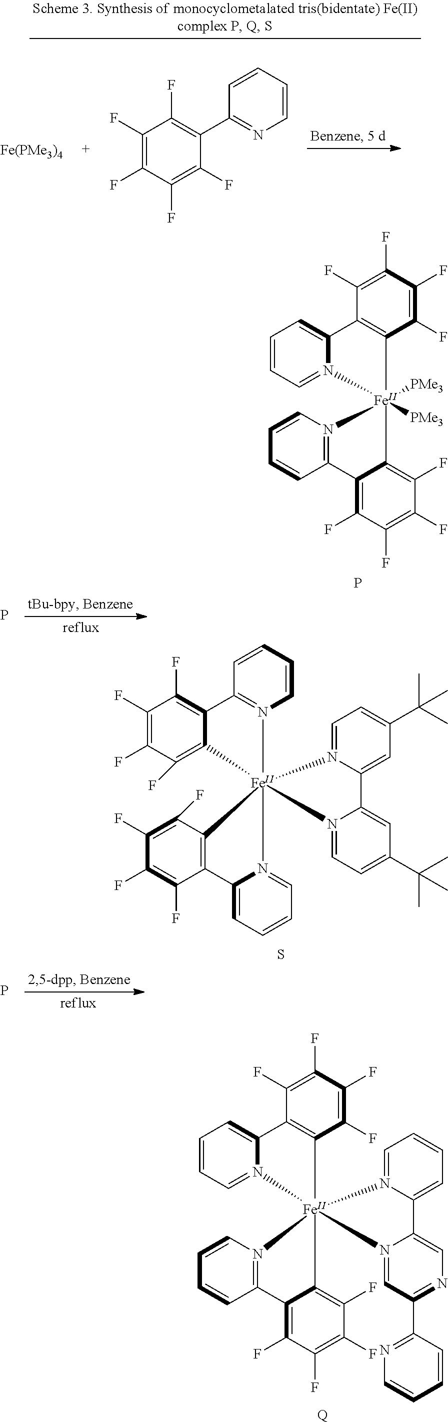

A deep-purple biscyclometalated bis(trimethylphosphine) Fe(II) complex P was obtained in varyingly low yields of 5-15% by reacting Fe(PMe₃)₄ with an excess of 2-(perfluorophenyl)pyridine (Scheme 3). P is air-sensitive and usually not obtained in a pure form. Single crystal of P for X-ray diffraction was obtained by laying pentane to a benzene solution. Synthesis of S, with the two PMe₃ displaced with a bpy, resulted in a mixture of products as indicated by NMR, probably a mixture of diastereomers (Scheme 3). Purification through column over alumina followed by recrystallization (benzene/pentane) of the major portion afforded dark single crystals suitable for X-ray diffraction. Mononuclear complex Q was obtained by replacing the PMe₃ ligands of P by a bridging 2,5-di (pyridin-2-yl) pyrazine (2,5-dpp) (Scheme 3). Complex Q was purified over a neutral alumina column. Complex Q is purple in solution and air-stable.

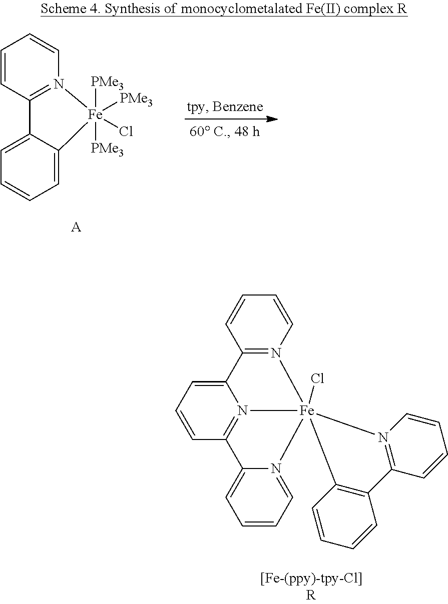

Complex A was used as a precursor for the synthesis of [Fe(II)-ppy-tpy-Cl] complex R via substitution of the three phosphines with a tpy ligand (Scheme 4). Complex A is a synthetic intermediate to a series of cyclometalated polypyridine Fe complexes. Complex R is brownish yellow in DCM and is slightly air-sensitive in both the solid and solution states.

### Results

### NMR Spectroscopy and X-Ray Structure

The ¹H NMR spectra of complexes C-R show signals in the typical diamagnetic region, indicating low-spin state for all the complexes. The ¹H NMR spectrum of complex C exhibits one high-field shifted aromatic proton signal slightly broadened at 5.80 ppm, indicating signals arising from shielding effect in related cyclometalated Fe(II) and Ru (II) complexes and assignable to the H-atom ortho to the cyclometalating donor. The ¹H NMR spectra of two stereoisomers of complex E (due to the asymmetry of the 5-Me-phen ligand) shows multiple aromatic signals, with a broad one discernable at 5.93 ppm, and four methyl signals in the range of 2.88-2.78 ppm.

P in benzene-d6 exhibits a multiplet at 19.64 ppm in the ³¹P NMR spectrum, while the PMe₃ protons also resonate a multiplet at 0.56 ppm in the ¹H NMR spectrum. Four proton signals were observed in the aromatic region, with a notable one on the high-field region at 5.87 ppm.

In the ¹H NMR spectrum of complex Q (CD₂Cl₂), the signals appear in the range of 8.92-6.81 ppm. The mononuclear formula was confirmed by HR ESI-MS (m/z found & Calc. 742.0815). The ¹H NMR spectrum of complex R (CD₂Cl₂) exhibits a high-field doublet (²J_{HH}=7.7 Hz) at 5.29 ppm and a low-field one (²J_{HH}=5.6 Hz) at 10.53 ppm. This signal pattern indicates that the molecule adapted a configuration with the cyclometalating C trans to the Cl atom and the N_{ppy} coplanar with the tpy. The 5.29-ppm signal is ascribable to the C—H ortho to the cyclometalating C atom and the 10.53-ppm one is assignable to the C—H ortho to the N_{ppy} atom.

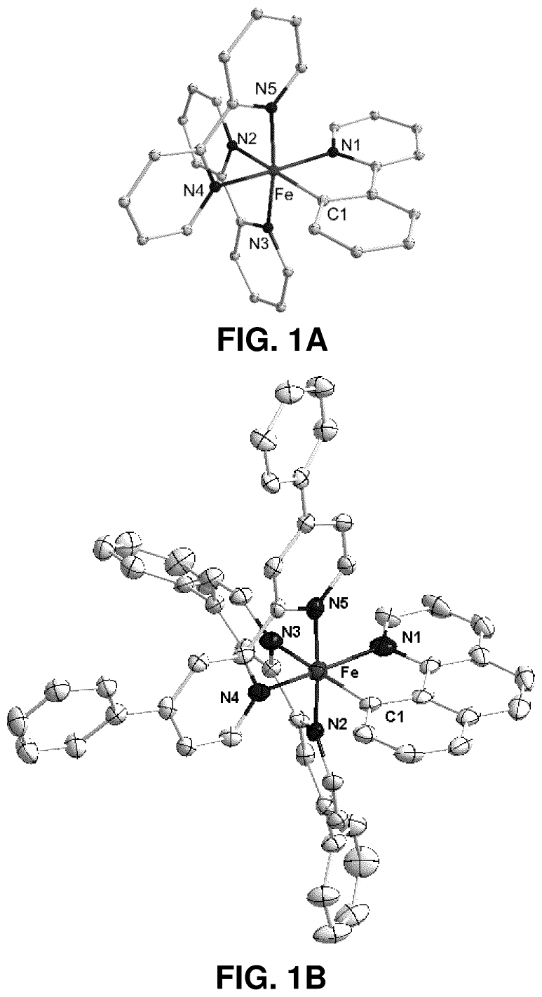

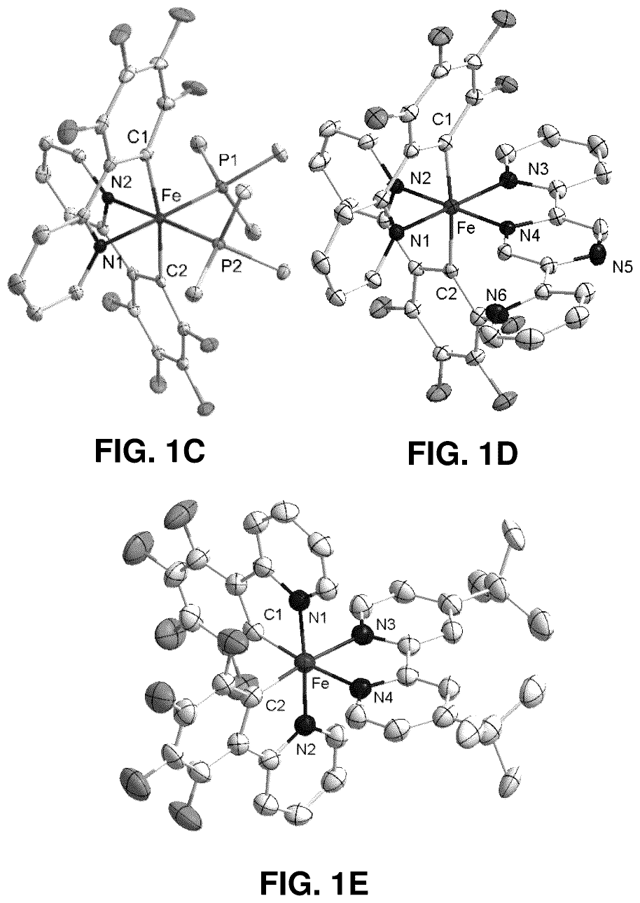

Single crystal of complex C suitable for X-ray diffraction was obtained by slow vapor diffusion of Et₂O into its CH₃CN solution. Complex C adopts a slightly distorted octahedral geometry (FIGS. 1A-1E), with the Fe—C bond length of 1.957 (4) Å that is comparable to the reported values of cyclometalated Fe(II) complexes. Complex P adopts a slightly distorted octahedral coordination with the two PMe₃ in mutual cis-positions (FIG. 1C). The two perflurophenyl carbanion donors occupy the two axial positions in a trans-fashion, with the two Fe—C bond lengths of 2.012 (2) and 2.023 (2) Å, which are at the long edge of typical Fe—C_{Ar} bond-length range, reflecting the mutual destabilizing trans-influence. The two Fe—N bond lengths are 2.002 (2) and 2.007 (2) Å. The non-coplanar arrangement of the two Ph^{5F}-Py ligands supports the high-field shift of the pyridine signal where shielding effect is commonly present.

Different from the structure of precursor complex P, complex S showed a structural arrangement where the two carbanion donors are cis to each other, while trans to the bpy-N donors (FIG. 1E). The Fe—C bond length of complex S is 1.949 (2) Å, dramatically shorter than those in complex P; the Fe—N_{bpy} bond length of complex S is 1.981 (2) Å, also relatively short. The axial Fe—N_{py} bond is 1.966 (2) Å in length.

Single crystals of Q were obtained by storing a Et2O solution layered with pentane at 0° C. for a week. The mutual trans arrangement of the two Fe—C bonds in that of complex A is maintained in the X-ray structure of complex Q (FIG. 1D), with the bond lengths of 2.021 (2) and 2.002 (1) Å, which are also comparable to those of complex A. The two Fe—N_{C\hat{ }N} bond lengths are 1.993 (2) and 2.001 (2) Å, respectively, which are slightly shorter than those of complex A. The two Fe—N_{2,5-dpp} bond lengths are 1.900 (1) and 1.950 (2) Å, indicating stronger x-backdonating interaction of the Fe center with the bipyridine moiety than with the pyridine_{C\hat{ }N}.

### UV-Vis Absorption and Electrochemistry

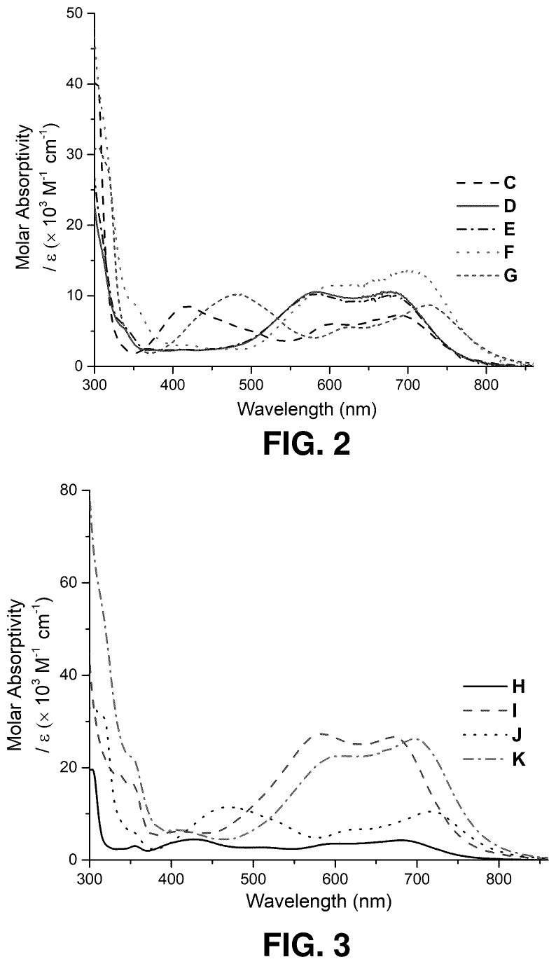

The UV-vis absorption spectra and data of the five Fe-ppy complexes in CH₃CN are shown in FIG. 2 and Table 1, respectively. Complex C showed three broad absorption bands at λₘₐₓ of 690, 602 and 418 nm in the visible region and ligand-centered absorptions at ˜300 nm. The lowest-energy band was red-shifted by 170 nm compared to that of [Fe(bpy)₃]²⁺, while the 418 nm band was shifted by ˜70 nm. These bands are even more red-shifted in the diPh-bpy complex F, showing absorptions at Amax of 730, 622 and 482 nm. The phen complexes D, E and G also showed broad and intense low-energy absorptions. The higher-energy absorption of the three phen complexes D, B and G at 412, 414 and 421 nm (ε=2360, 2390 and 2990 L mol⁻¹ cm⁻¹), respectively, was notably lower in intensity than that of the two bpy complexes C and F (at 418 and 482 nm, ε=8500 and 10140 L mol⁻¹ cm⁻¹, respectively).

TABLE 1
Spectroscopic data of [Fe(bpy)₃]²⁺ and exemplary
cyclometalated tris-bidentate Fe(II) complexes in MeCN.

| Complex | λₘₐₓ [nm] (ε [10³ L mol⁻¹ cm⁻¹]) |
|---|---|
| [Fe(bpy)₃]²⁺ * | 520(7.98), 486(6.88), 349(6.03), 298(62.2), 248(27.5) |
| C | 690(7.23), 602(6.00), 505(4.83, sh), 418(8.50), 303(40.3), |
| | 298(40.4) |
| D | 677(10.45, br), 581(10.55), 412(2.36), |
| | 375(2.32), 338(5.32, sh) |
| E | 682(10.10), 582(10.25), 414(2.39), 347(4.53, sh), |
| | 335(6.10, sh), 294(32.2, sh), 271(68.3) |
| F | 704(13.50), 600(11.39), 421(2.99), 354(8.47, sh), |
| | 321(34.6), 283(81.2), 280(78.9) |
| G | 730(8.65), 622(5.52), 482(10.14), 317(28.12), 306(30.95) |
| H | 680(4.27), 597(3.56), 513(2.72), 427(4.48), 356(3.03, sh), |
| | 304(19.55), 296(19.76) |
| I | 671(26.63), 581(27.27), 407(6.18), 353(16.41), |
| | 338(18.31, sh), 270(176.44), |
| J | 716(10.47), 617(6.54), 467(11.44), 355(5.92, sh), |
| | 317(30.97), 305(33.28), 263(73.41) |
| K | 697(26.20), 596(22.32), 408(6.52), 353(22.08), |
| | 315(55.34, sh), 280(157.82) |
| O | 673(6.66), 598(6.25), 514(3.66), 462(4.86), |
| | 412(8.17), 324(15.68, sh), 298(45.41) |
| Q | 744, 535, 499, 423, 339(sh), 323 |
| R | 998, 805, 689, 476, 373, 328(sh), 313 |
| S | 295, 441, 524(sh), 596, 753 |

* Y. Liu, et al., Chem. Eur. J. 2015, 21, 3628-3639

The UV-vis absorptions of the four Fe-BQ complexes are similar in pattern to those of the Fe-ppy complexes (FIG. 3). The two lowest-energy bands of the Fe-BQ complexes are marginally hypsochromically shifted than those of the Fe-ppy complexes. Slightly hypsochromic shifts were also observed for the higher-energy band at 409˜468 nm for complexes I, J, and K compared to those of the corresponding Fe-ppy complexes D, F and G, respectively, while a small bathochromic shift was observed for complex H compared to complex C. The much weaker absorptions at these wavelengths for the phen complexes than those of the bpy derivatives were in line with the observations for the Fe-ppy analogues.

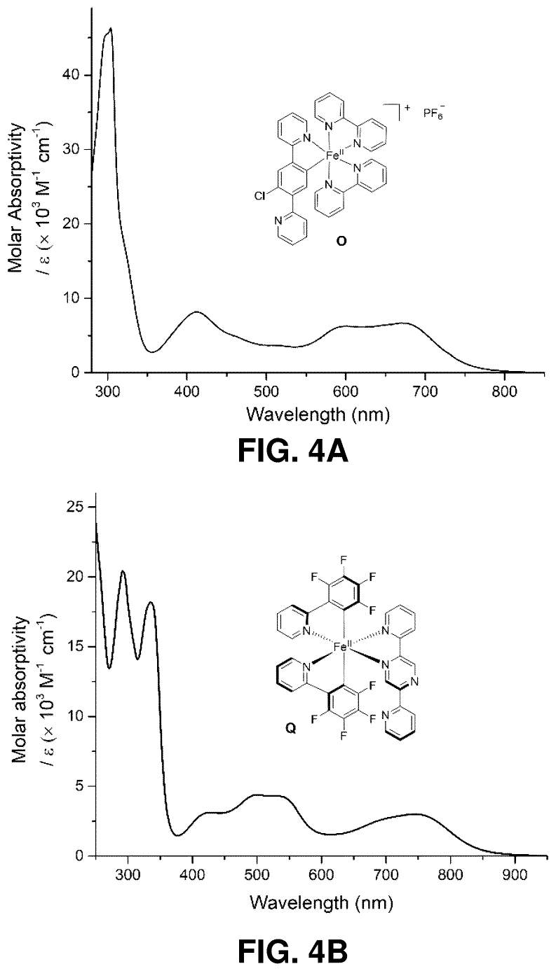

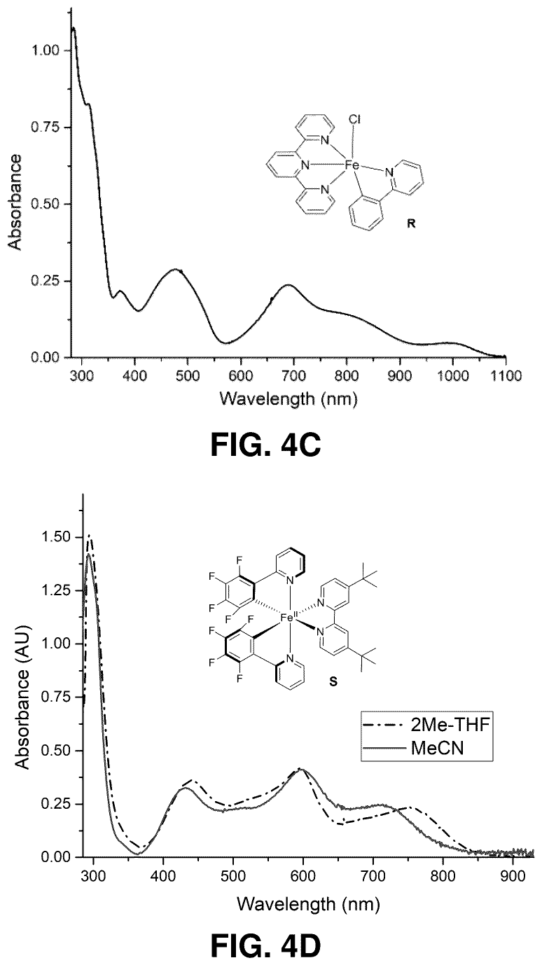

Complex O showed an absorption profile similar to that of complex C (FIG. 4A). The major bands of complex O are slightly hypsochromically shifted compared to those of complex C, with the largest shift of 17 nm for the lowest-energy band. Complex Q showed four bands in the visible range, with the lowest-energy one broad at 744 nm (FIG. 4B). Complex R showed four absorption bands in the visible and near-IR range at λₘₐₛ=998, 805, 689, 476 nm, with the low-energy absorption extending to 1100 nm (FIG. 4C). Monocyclometalated tris(bidentate) Fe(II) complexes C—O showed panchromatic absorptions, with the two low-energy bands spanning 500-800 nm, and with the lowest-energy bands shifted by 170-180 nm compared to that of [Fe(bpy)₃]²⁺. The shapes of the three absorption bands of complex C and their relative intensities are comparable to those of [Ru(ppy)(bpy)₂]⁺, only broader and red-shifted than the latter. The two broad absorptions at 690 and 602 nm were thus assigned to Fe→bpy MLCT transitions. The broad band at 418 nm may be assigned to Fe→ppy MLCT transitions, in reference to [Ru(ppy)(bpy)₂]⁺. These assignments should be applicable to the absorptions of the other complexes D-O. Notably, in the phen-derived complexes, e.g., D and G, the much weaker absorption at ˜420 nm relative to the lower-energy band is consistent with those of the cyclometalated Ru-phen analogues. Complex S exhibited panchromatic absorption in MeCN with λₘₐₓ=710, 597 and 432 nm with the lower-energy absorption extending to 850 nm. The lowest-energy band showed a notable bathochromic shift in 2Me-THF with λₘₐₓ=750 nm.

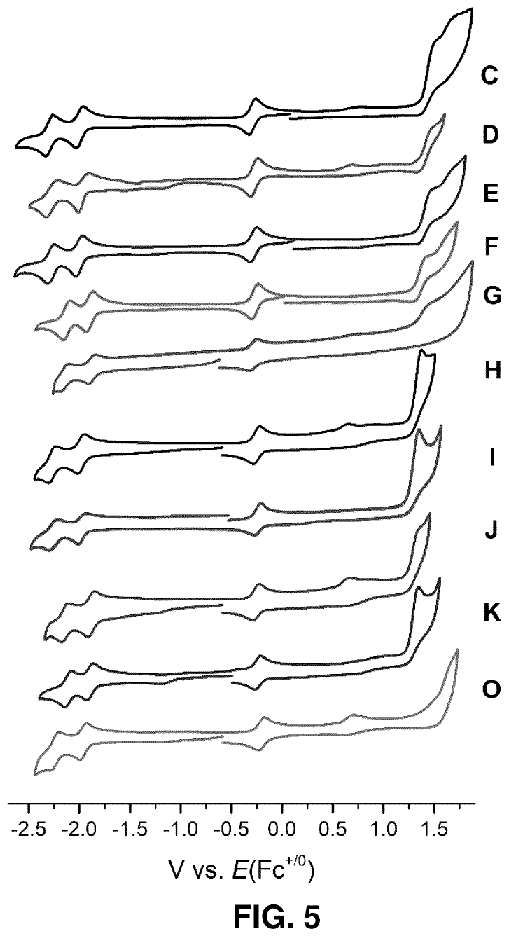

Electrochemistry of the mononuclear complexes in 0.1 M TBAP CH 3CN solution was studied using cyclic voltammetry (“CV”). The voltammograms and CV data are shown in FIG. 5 and Table 2, respectively. As shown in FIG. 5, there were three reversible redox waves, ascribed to Fe(II)-to-Fe(III) oxidation and two ligand reductions. The E_{1/2}(Fe^{III/II}) of complex C (i.e., ˜0.28 V vs E (Fc^{+/0})) is 0.96 V more cathodic than that of the benchmark [Fe^{II}-(bpy)₃]²⁺ complex (Y. Liu, et al., Chem. Eur. J. 2015, 21, 3628-3639). This potential shift is more drastic than that between the Ru congeners (P. G. Bomben, et al., Inorg. Chem. 2009, 48, 9631-9643), demonstrating a remarkable donating effect of the cyclometalating ligand on Fe(II) center. The two reduction potentials of complex C were more negative than those of [Fe(bpy)₃]²⁺, but to a less extent than the Fe^{III/II} couple, indicating a more pronounced HOMO-than LUMO-destabilization. This is consistent with the drastically bathochromically shifted absorption of complex C than [Fc(bpy)³]²⁺.

The other four Fe-ppy complexes showed redox waves at potentials almost identical to those of complex C. The two ligand reduction potentials are anodically shifted by ˜0.1 and 0.15 V for complexes F and G, respectively, which is expected due to the x-delocalizing phenyl substituents. Surprisingly, the two ligand reduction peaks of complex D are pseudo-reversible under the same conditions, with the wave-shape scan-rate dependent.

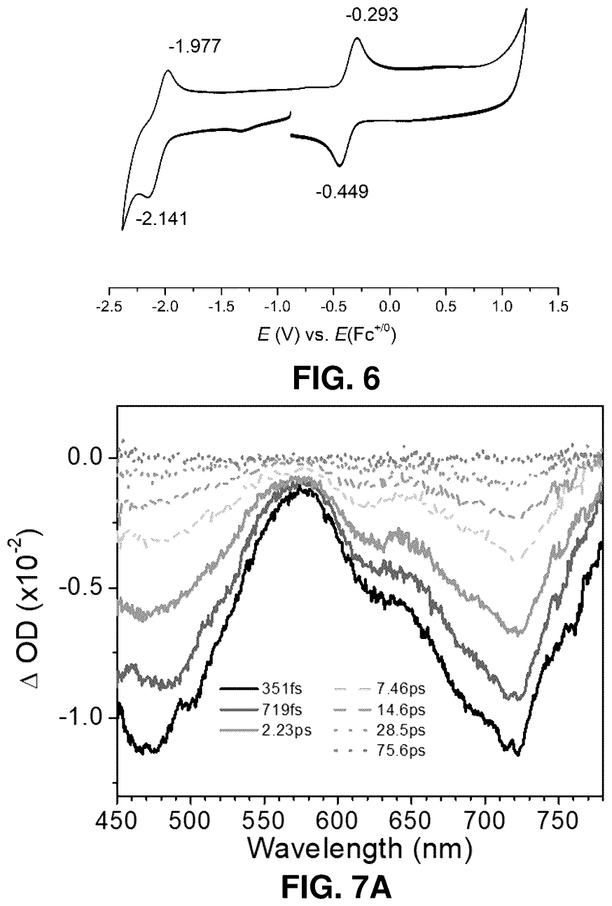

The series of Fe-BQ complexes showed similar redox waves at comparable potentials, only that the E_{1/2} (Fe^{III/II}) and Eₚₐ(Ox. I) appeared slightly more negative by ˜20-40 mV and ˜70-150 mV, respectively, than those of the Fe-ppy analogues. One irreversible oxidation wave at E=1.41-1.52 for the ppy complexes and 1.34-1.37 V for the BQ complexes was observed (FIG. 5), which is likely associated with Fe(III)-to-Fe(IV) oxidation, reminiscent of those of the bis (tridentate) monocyclometatated Fe(II) complexes (Z. Tang, et al., Organometallics 2020, 39, 2791-2802). The voltammogram of complex O is similar to that of complex C, only that the E_{1/2} (Fe^{III/II}) is ˜0.08 V anodically shifted than that of the latter (FIG. 6), which indicates a lowering of HOMO of complex O comparable to complex C, consistent with the observed absorption-band shift (FIGS. 2 and 4A, Table 1). The voltammogram of complex Q showed two reversible redox couples with relatively large ΔEₚ at E_{1/2}=−0.37 and −2.06 V vs Fc⁺/Fc. The former is ascribed to the Fe^{III/II} redox reaction. The relatively negative value is probably the direct result of the biscyclometalated structure.

TABLE 2
CV data of the cyclometalated tris-bidentate Fe(II) complexes in MeCN.

| | E_{1/2}(Red. II) | E_{1/2}(Red. I) | E_{1/2}(Fe^{III/II}) | Eₚₐ(Ox. | Eₚₐ(Ox. |
|---|---|---|---|---|---|
| complex | (ΔEₚ/mV) | (ΔEₚ/mV) | (ΔEₚ/mV) | I) | II) |
| [Fe(bpy)₃]^{2+[16]} | −1.94 | −1.75 | 0.68 | / | / |
| C | −2.29 (67) | −1.99 (66) | −0.28 (65) | 1.52 | 1.65 |
| D | −2.26 (106)* | −1.98 (96)* | −0.27 (72) | 1.48 | / |
| E | −2.28 (65) | −2.00 (65) | −0.28 (63) | 1.48 | 1.67 |
| F | −2.13 (59) | −1.90 (62) | −0.27 (62) | 1.41 | 1.62 |
| G | −2.15 (77) | −1.87 (58) | −0.28 (82) | 1.45 | / |
| H | −2.27 (70) | −1.98 (64) | −0.25 (60) | 1.37 | / |
| I | −2.26 (66) | −1.98 (66) | −0.23 (62) | 1.35 | / |
| J | −2.14 (61) | −1.88 (57) | −0.25 (54) | 1.34 | / |
| K | −2.11 (62) | −1.90 (60) | −0.24 (53) | 1.35 | / |
| O | −2.24(62) | −1.96 (60) | −0.20 (65) | 1.51 | / |
| Q | / | −2.06(164) | −0.37(156) | / | / |

*pseudo-reversible

The electrochemical studies of the three complexes revealed a cathodic shift of the E_{1/2}(Fe^{III/II}) (i.e., at ˜0.28 V) by ˜0.95 V compared to that of [Fe(bpy)₃]²⁺, which was also ˜0.17 V more negative even than that of the monocyclometalated bis (tridentate) [Fc(ppy)(bpy)₂]⁺ (Z. Tang, et al., Organometallics 2020, 39, 2791-2802; J. Steube, et al., Chem. Eur. J. 2019, 25, 11826-11830). The ligand reduction potentials were negatively shifted by only ˜0.25 V for complex C compared to [Fe(bpy)₃]²⁺, and compared to [Fc(ppy)(bpy)₂]⁺ (i.e., shifted by 0.03 V). These results demonstrate destabilized HOMOs and a narrowed HOMO-LUMO gap for the cyclometalated complexes than for [Fc(bpy)₃]²⁺, which is in agreement with the red-shifted visible absorptions compared to the latter. These results also demonstrate a strong σ-donating ability of the cyclometalating unit.

### Femtosecond Transient Absorption Spectroscopy

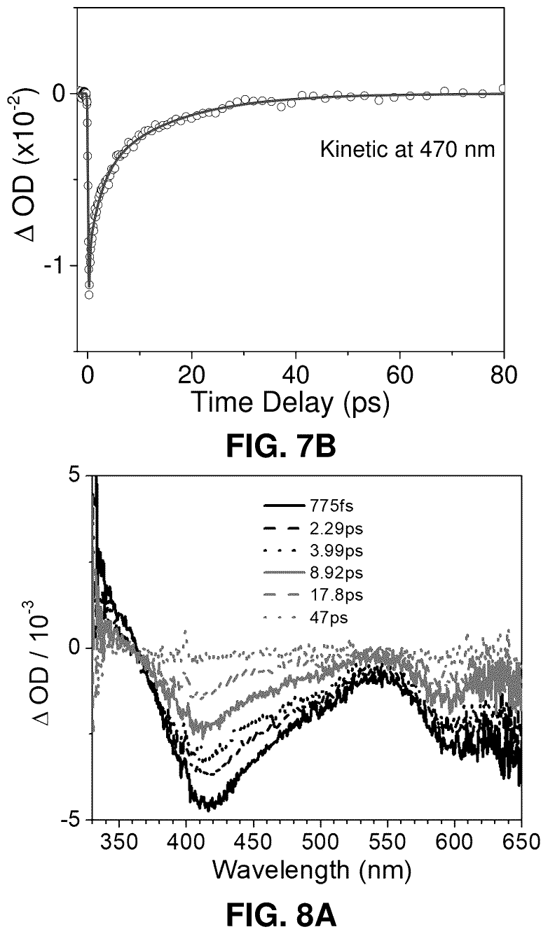

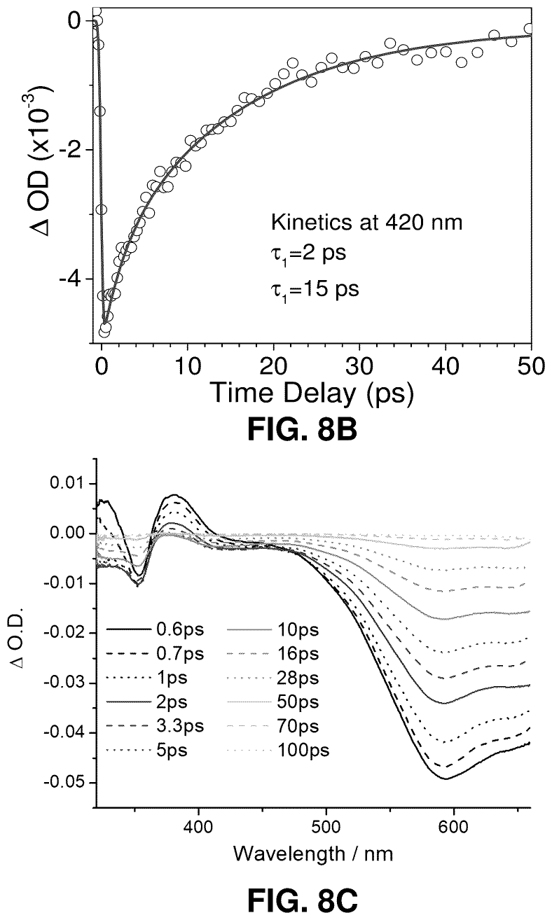

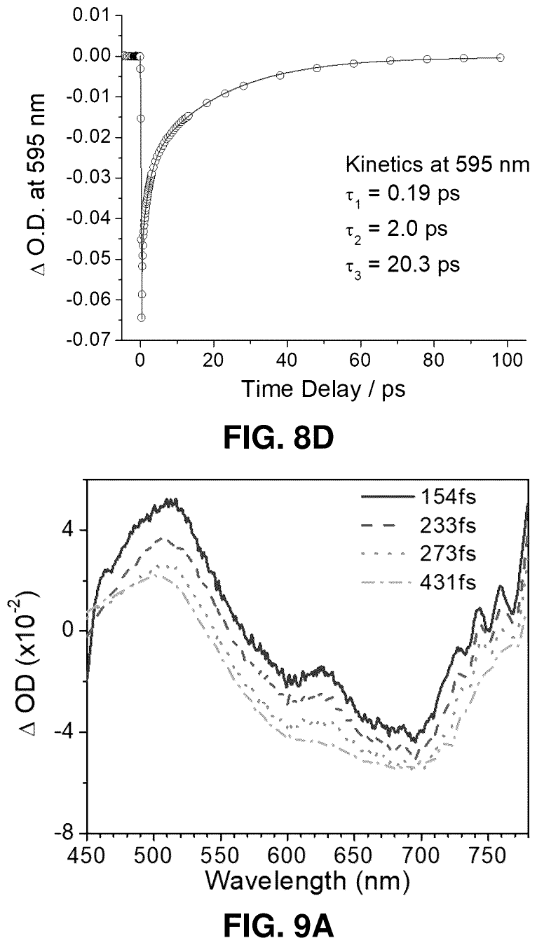

The excited-state kinetics of complexes C, F and K were studied using fs-TA measurement in CH₃CN at 293 K. For the bpy complexes C and F, upon excitation at 266 and 455 nm, respectively, only bleaching of the ground-state absorption (GSA) at 420˜470 and >600 nm were observed in the spectral range of 450-850 nm ˜350 fs after the excitation (FIG. 7A for F, FIG. 8A for C). Kinetic fittings at 420 nm for complex C (FIG. 8B) and 470 nm (FIG. 7B) for complex F gave time constants of 2 and 15 ps for complex C and 1.5 and 12.1 ps for complex F. For complex K, upon excitation at 690 nm at 293 K, absorption features were observed at 353 and 380 nm in the early stage (FIG. 8C). The spectral evolution was dominated by bleaching of the GSA in the >500 nm region. Kinetic fittings at 595 nm gave three time constants of 0.19, 2.0, and 20.3 ps (FIG. 8D).

For complex C, upon excitation at 266 nm, the major feature in the TA spectra after 775 fs was the ground-state bleaching at the wavelengths of ˜420 and 600 nm (FIG. 8A). This excited-state absorption (ESA) feature decayed to a large extent along with the recovery of the GSA. Fitting the kinetics at 420 nm (FIG. 8B) yielded two time components of 2 ps and 15 ps, each is likely associated with a triplet excited state, as a singlet excited state is expected to be much shorter-lived and a 5MC one is expected to be high in energy for a cyclometalated structure.

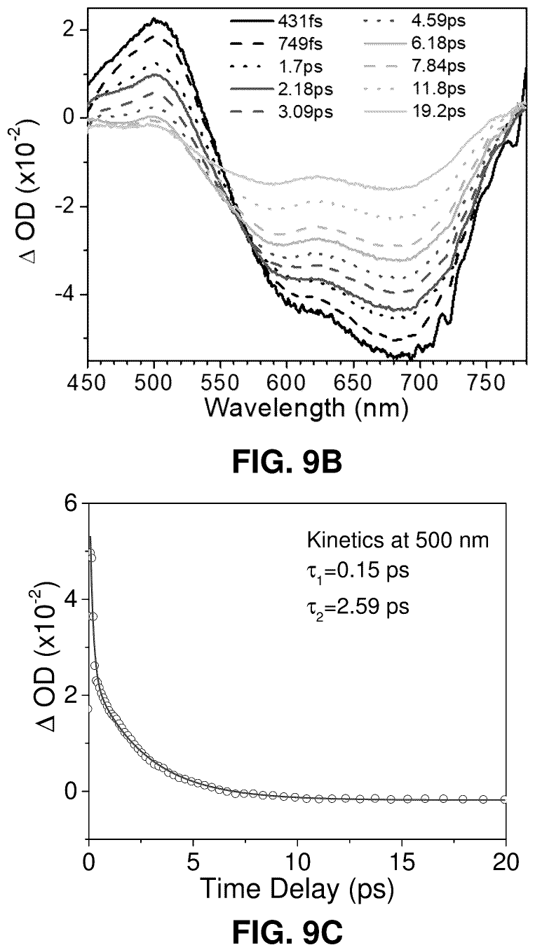

For complex G, a prominent ESA at 515 nm was immediately observed in 154 fs upon excitation at 400 nm, which decayed within several picoseconds with recovery of the GSA (FIGS. 9A and 9B). Kinetic fitting of the 500 nm feature yielded two time constants of 0.15 and 2.59 ps (FIG. 9C). The former should be associated with a singlet-to-triplet intersystem crossing, while the latter might be associated with a triplet excited state.

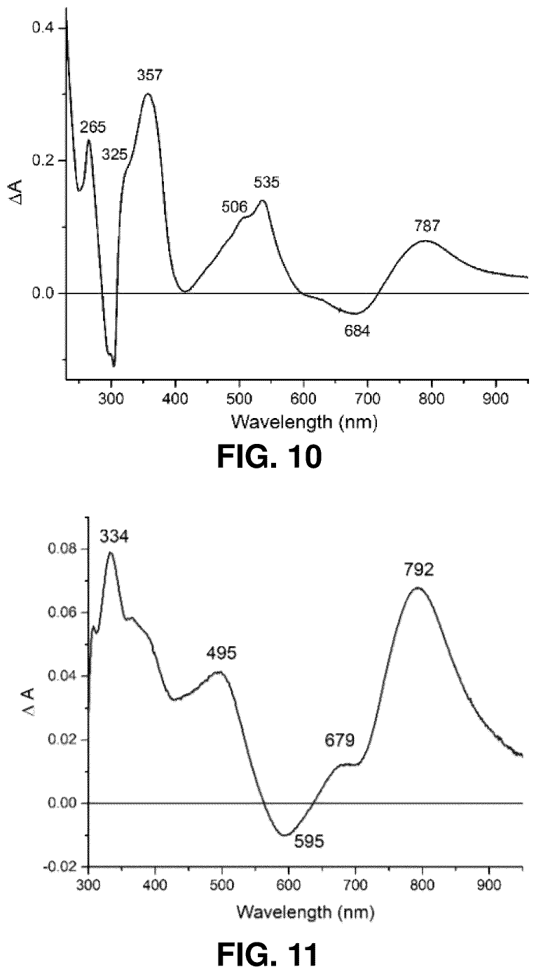

The absence of absorptive features in the fs-TA spectra of complex C, except for the probably artificial feature at 2˜340 nm, precluded a more detailed assignment of the triplet excited states (FIGS. 8A and 10). For complex G, the longer 2.59-ps time component may be ascribed to a triplet excited state. The ESA at 500 nm associated with this excited state bears a high resemblance to the absorption at 495 nm in the reductive differential spectrum (FIGS. 9A-9B and FIG. 11), which indicates that this excited state is of ³MLCT character. The 2.59-ps lifetime is one order longer than those typical Fc(bpy)₃]²⁺ complexes, which may be attributed to both the strong-field effect of the cyclometalating donor and the low-lying π*(diPh-phen).

### Spectroelectrochemistry

UV-vis spectroelectrochemical (SEC) studies were performed for complexes C and G in MeCN. Upon one-electron oxidation, the MLCT bands of the complexes disappeared, in accordance with the depletion of the Fe(II) centers. Upon one-electron reduction, broad new absorptions were observed for the two complexes (FIGS. 10 and 11). For complex C, three major absorptions at λₘₐₓ˜787, 535 (506) and 357 nm appeared (FIG. 10). These features may be assigned to absorptions of a bpy ligand radical anion. Intensity enhancement of these absorptions was observed upon further one-electron reduction, indicating generation of a second bpy ligand radical anion. For complex G with phen ligands, qualitatively similar SEC spectral changes were observed, where three major bands were noted upon one-electron reduction with the middle-energy ones appearing at 495 nm (FIG. 11).

### Example 2. Self-Assembly of Exemplary Cyclometalated Fe(II) Complexes as Photosensitizer for Effective Photothermal Therapy

Exemplary cyclometalated polypyridine Fe(II) complex as photothermal transduction agents (PTAs) were demonstrated. This type of complexes have high structural robustness due to strengthened Fe-L bonds (where enhanced metal-to-ligand back-donation may play a role), and good visible-to NIR-absorptivity due to narrowed energy gap arising from higher metal-based HOMOs. In addition, the cyclometalated Fe (II) photosensitizer combines the flexibility of metal supramolecular self-assembly and the advantage of strong near-infrared (NIR) absorbance with high photo-heat conversion efficiency of 60.6% (where commercial gold nanorods: 21.0%, Cu2-xSe: 22% and Cu₉S₅: 25.7%) and superior photothermal stability under 808 nm laser irradiation. The data demonstrated excellent tumor-targeted photothermal efficacies and good biosafety in vitro and in vivo using supramolecular photosensitizers (also referred to herein as “metallosupramolecular particles,” “Fe nanoparticles,” or “Fe NPs”) formed by the cyclometalated polypyridine Fe(II) complex disclosed herein.

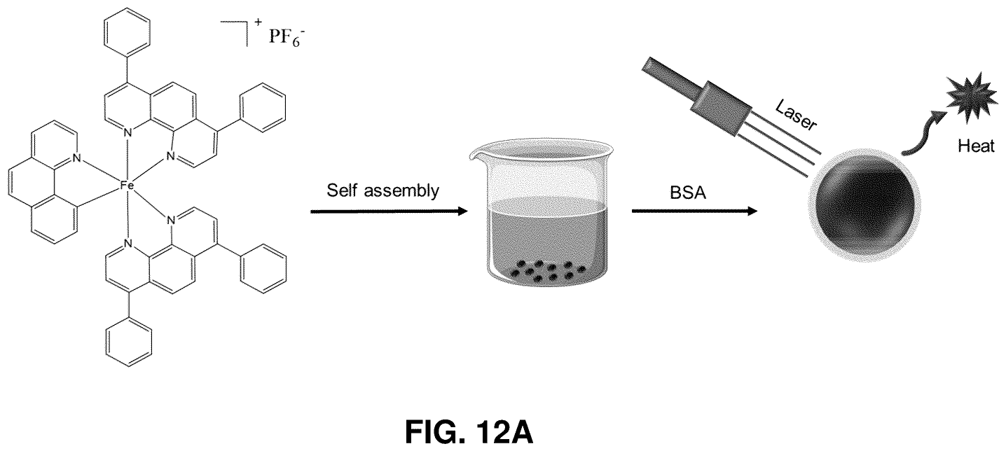

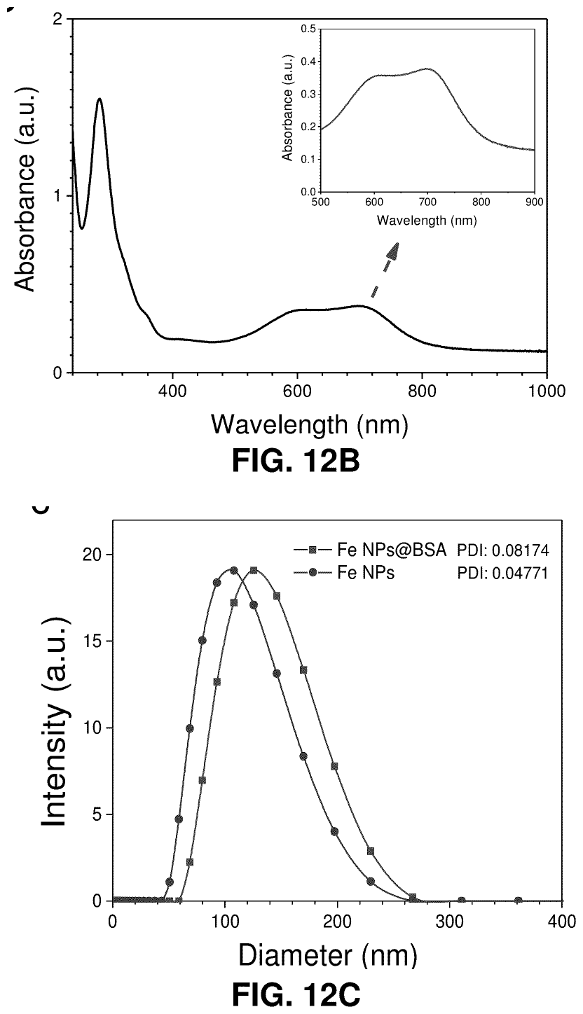

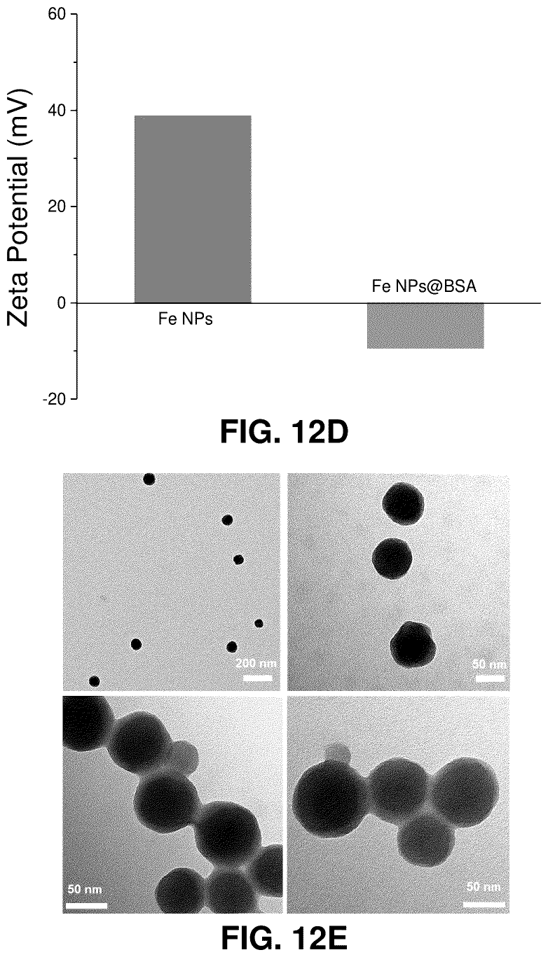

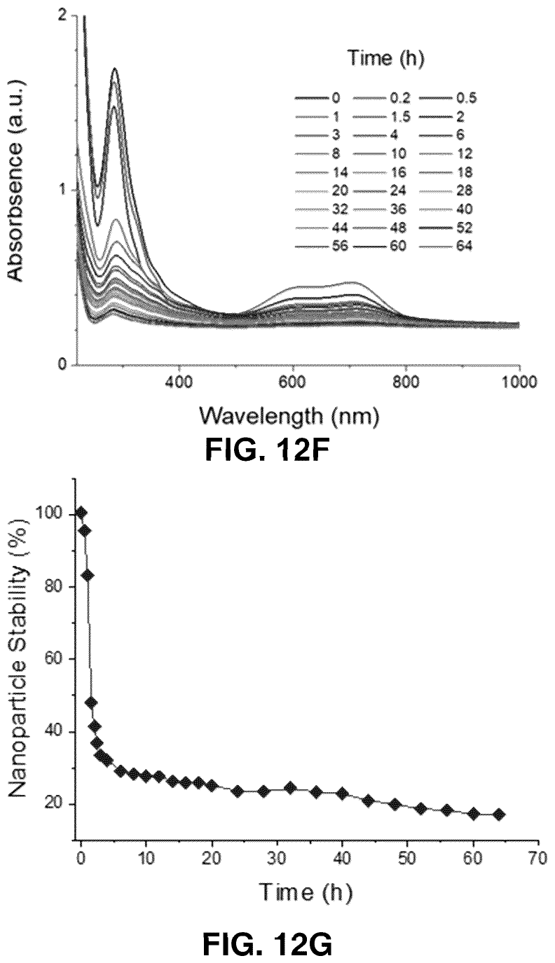

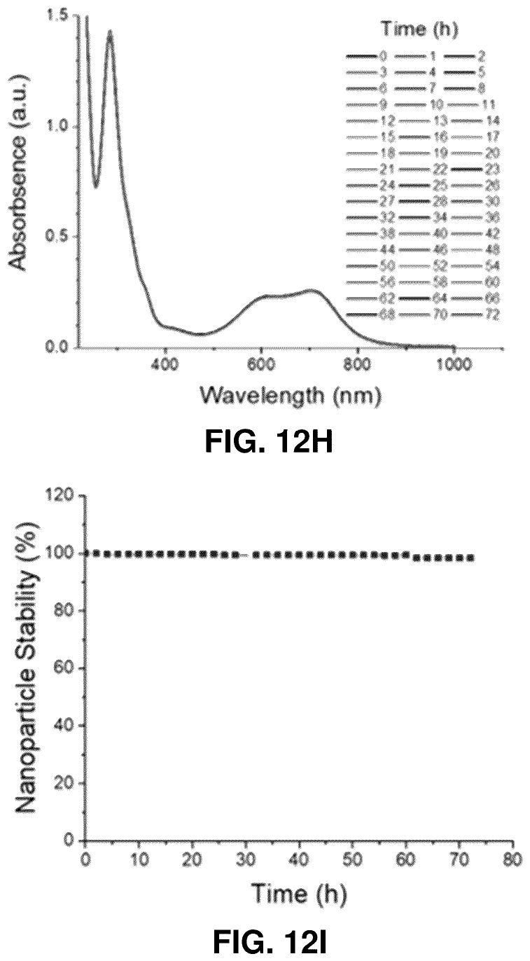

The exemplary benzo[h]quinoline-10-yl bis-bathophenanthroline Fe(II) complex (Fe(II)-BQ-Bphen₂) showed NIR absorbance, and can self-assemble into metallosupramolecular particles (i.e., Fe NPs) in water system through noncovalent interactions (FIG. 12A, left and middle). To enhance the tumor accumulations and biocompatibility of the metallosupramolecular particles in vivo, bovine serum albumin (BSA) was used to coat the Fe NPs, forming Fe(II)-BQ-Bphen2@BSA nanoparticles (also referred to herein as “Fe NPs@BSA,” “Fe-BB nanoparticles,” “Fe-BB NPs,” or “BSA coated Fe NPs”)) (FIG. 12A, middle and right). Data demonstrated that the particles act as a highly efficient PTT agent. In anticancer efficacies, Fe-BB NPs was used to treat with 4T1 tumor bearing balb/c mice to explore PTT efficacies in vivo.

### Materials and Methods

### Preparation of Fe NPs and Coated Fe NPs

To produce Fe NPs of the Fe complexes, 2 mg Fe complex was dissolved in 0.5 mL DMSO. Then, the solution was added to 10 mL deionized water dropwise and mixture was stirred at room temperature for 4 h. Subsequently, the obtained product was collected and washed three times by using the Amico tube (10 kda).

To produce Fe NPs coated with BSA, the prepared Fe nanoparticles and BSA (w:w=1:0.5) were added to 15 mL phosphate buffer (Na₂HPO₄—NaH₂PO₄, pH=8.0) under ultrasound at room temperature for 4 h Finally. The product was conventionally centrifuged (13,000 rpm, 10 min) and washed three times with distilled water. The collected materials were stored at dark environment for further use.

The Fe NPs and Fe-BB NPs were characterized using TEM, dynamic light scattering, zeta potential, FTIR, and XRD.

### Cell Culture

Human NCI-H460 lung cancer cells and mouse 4T1 breast cancer cells were purchased from the American Type Culture Collection (ATCC). Cells were routinely tested for mycoplasma contamination using MycoSET Mycoplasma real-time PCR detection Kit (Life Technologies, Foster City, CA, USA). NCI-H460 cells and 4T1 cells were cultured in Roswell Park Memorial Institute-1640 (RPMI-1640) with 10% (v:v) fetal bovine serum (FBS), 100 U mL-1 penicillin and 100 μg mL-1 streptomycin in an incubator at 37° C. under an atmosphere of 5% CO₂ and 90% relative humidity. A549 cells were cultured in Dulbecco's Modified Eagle's Medium (DMEM) supplemented with 10% (v:v) FBS, 100 U mL-1 penicillin and 100 μg mL-1 streptomycin in the same incubator as NCI-H460 cells. 0.25% trypsin was used to digest and subculture these cells.

### In Vitro Photo-Cytotoxicity of Fe NPs@BSA

NCI-H460 cells or 4T1 cells were planted in a 96-well plate (8×10³ cells/well). After incubation for 24 h, the culture medium was replaced with fresh ones containing Fc nanoparticles at final concentrations of 0-100 μg/mL. After incubation for overnight, each well in half of the cell plate was irradiated under the 808 nm laser (0.5 W/cm² or 1 W/cm²) for 10 min as PTT group, and other wells were in dark as control group. After another 24 h incubation, 10 μL CCK-8 (WST-8) was added to each well and continue to incubate for another 1 h. Cell viability was determined as the absorbance at 450 nm using a Thermo Scientific Varioskan™ Lux multimode microplate reader.

### Cellular ROS Measurement

NCI H460 cells or 4T1 cells were seeded in 96 plates (8×10³ cells/well) and incubated for 24 h. These cells were divided into 6 groups: control with/without laser and Fc NPs@BSA (25 μg/mL or 50 μg/mL) with/without laser. After 6 h incubation, the PTT groups of this plate were irradiated for 10 min/well by using an 808 nm laser (1 W/cm²). Finally, 10 μL ROS probe-H2DCFDA was added to each well (finally 5 M/well) and the fluorescence of the plate was detected by microplate reader (Ex 490 nm, Em 525 nm) after incubating the plate for 60 min.

### Immunofluorescence Staining for DNA Damage.

2×10⁴ cells were seeded on glass coverslips. After incubation for 24 h, these cells were treated with Vehicle or Fe NPs@BSA (50 μg/mL) for 4 h in an incubator, following by irradiation of 10 min with an 808 nm laser (1 W/cm²) for PTT groups. For γH2AX staining, cells were fixed in 4% paraformaldehyde/PBS for 15 min at room temperature, permeabilized with 0.3% Triton X-100/PBS for 15 min on ice, then blocked in 0.2% BSA/PBS for 1 h at room temperature. Finally, the cells were stained with γH2AX (Cell Signaling, #2577L, 1:100 dilution, at 4° C., 2 h) and labeled with secondary antibodies (Invitrogen, Alexa Fluor® 568 dye, 1:200 dilution, 2 h, RT). For DNA staining, cells were stained with DAPI (1:400 dilution, at 4° C., 15 min). Coverslips were then visualized and recorded by Micro Confocal High-Content Imaging (ZEISS).

### Cell Apoptosis Study by Flow Cytometry

4T1 cells were seeded in 6 plates (3×10⁵ cells/well). These cells were treated with 25, 50, or 100 μg/mL Fe NPs@BSA, or PBS for 4 hours, followed by irradiation under an 808 nm laser (1 W/cm²) for 10 min as photo-treatment or left in dark as control. Then, cells were collected in 1.5 mL tubes. Cell apoptosis was determined by flow cytometry using an Annexin V-FITC/PI assay kit at room temperature for 15 min, according to the manufacturer's protocol. The fluorescence of cells was measured using flow cytometer. The results were presented as a percentage of normal and apoptotic cells at various stages.

### Photothermal Effect of Nanoparticles

The photothermal heating curves were obtained by monitoring the temperature changes of sample solutions in a 1.5 mL Eppendorf tube under the irradiation of an 808 nm laser at different power densities (0.2, 0.5, 1.0 W/cm²) or using different concentrations of sample solution. The laser was provided by a fiber-coupled continuous semiconductor diode laser and the temperature was recorded by a fixed-mounted thermal imaging camera (FLIR A300-series). The photothermal conversion efficiencies (n) of Fe NPs@BSA were calculated using the method of Roper, et al., The Journal of Physical Chemistry C, 2007, 111 (9), 3636-3641.

Specifically, Following Roper's method, the photothermal conversion efficiency (η) of Fe NPs was calculated using the following equation

$$η = \frac{hS ( {T}_{max} - {T}_{surr} ) - {Q}_{Dis}}{I ( 1 - {10}^{- {A}_{808}} )} \qquad ( 1 )$$

where h (mW/(m²·° C.)) is heat transfer coefficient, S (m²) is the surface area of the container, Tmax is the equilibrium temperature, and Tsurr is ambient temperature of the surroundings. In this experiment, Tmax−Tsurr was 51.3° C. according to FIG. 13C. The Q_{Dis} (mW) expresses the heat from light absorbed by the cuvette sample walls and it was measured to be 8.80 mW using a quartz cuvette cell containing aqueous samples without Fe NPs. I is the incident laser power (1 W) and A808 is the absorbance (0.579) of supra-CNDs at 808 nm.

To get the hS, θ is introduced using the maximum system temperature, Tmax

$$θ = \frac{T - {T}_{surr}}{{T}_{max} - {T}_{surr}} \qquad ( 2 )$$

and a sample system time constant τs

$${τ}_{s} = \frac{{\sum }_{i} {m}_{i} {C}_{p , i}}{hS} \qquad ( 3 )$$

according to the following expression

$$t = - {τ}_{s} ln ( θ ) \qquad ( 4 )$$

τs was determined to be 473.13 s thus hS was deduced to be 9.91 mW/° C. (substituted m=1 g, C=4.2 J/g·K in Equation (3)).

Finally, the photothermal conversion efficiency (n) of Fe NPs was calculated to be 60.6% from equation (1).

### In Vivo Photothermal Imaging (PTI)

4T1 tumor-bearing mice model was established by injecting 1×10⁷ 4T1 cells into the hind limb of each female BALB/c mouse (˜20 g, purchased from Charles River Laboratories (Wilmington, MA)). After the mean volume of the tumors reached about 100 mm³, two experienced researchers randomly divided the mice into three groups (n=3 per group). The tumor-bearing mice were injected with 100 μL of PBS (group 1) or 10 mg/kg Fc NPs@BSA (group 2) through the tail vein. After 1 h of injection, the mice were irradiated with the 808 nm laser at 0.5 W/cm² for 6 min. During irradiation, infrared thermal imaging camera (FLIR A300-series) was used to monitor the temperature change of the tumor sites. Group 1 was taken as the control. The Administrative Committee on Animal Research in The University of Hong Kong approved the protocols for all animal experiments.

### Tumor Therapy Study In Vivo

Female BALB/c mice were purchased from Charles River Laboratories (Wilmington, MA). The mice were maintained according to the requirements of the Laboratory Animal Unit (LAU) of The University of Hong Kong (HKU) and animal experiments were conducted based on the guidelines approved by the Committee on the Use of Live Animals in Teaching and Research of HKU. For tumor establishment, 1×10⁷ 4T1 cells in 100 μL PBS were injected into the back flank of each mouse by subcutaneous injection. When the tumor size reached approximately 60-100 mm³ (designed as day 0), the treatment was performed. Two experienced researchers randomly divided the mice into four groups (n=5 per group for 4T1 tumour), which were injected, by intravenous tail vein administration, with 100 μL PBS, without and with 808 nm laser irradiation (as two blank controls, group 1 and group 2), 10 mg/kg Fe nanoparticles (as thermal therapy control, group 3), or 10 mg/kg Fe nanoparticles with an 808 nm laser irradiation (as thermal therapy group, group 4), on days 1, 3 and 5. Groups 2 and 4 were treated with an 808 nm laser at 0.5 W/cm² for 6 min at tumor sites after 1 h of injection. The body weight and tumor size of each mouse were recorded every other day.

The body weight and tumor size of each mouse were recorded thrice per week and the tumor volume (V) was calculated by the following equation:

$$V = {ab}^{2} \times 0.5$$

where a and b are the longest and the shortest diameters of the tumor, respectively. The mice were humanely killed after 12 days of treatment and all the tumors were collected for further analysis.

### The Liver/Kidney Function and Heamotoxicity Analyses.

Two experienced researchers randomly divided the BALB/c mice into 3 groups (n=4 per group), which were intravenously injected with 100 μL PBS (as control, group 1), 100 μL 10 mg/kg Fe nanoparticles (group 2), or 100 μL 20 mg/kg Fe nanoparticles (group 3). After 12 days, the blood of each mouse was taken and analyzed using a blood cell analyzer (BC-31 s, Mindray) and test kit (Beyotime Biotechnology).

### Results

### Synthesis and Characterization of Fe-BB NPs

As the transmission electron microscopy (TEM) revealed, the morphology size of Fc(II)-BQ-Bphen2 complex and Fe-BB NPs were approximately 80 nm and 100 nm (FIG. 12E). The organic layer was observed by TEM image on the surface of nanoparticles due to introduction of the BSA (data not shown). The hydrodynamic diameter of Fe(II)-BQ-Bphen2 complex nanoparticles was determined to be approximately 90 nm using dynamic light scattering (DLS) (FIG. 12C). After coating with BSA, the hydrodynamic size of the nanoparticles increased from 90 nm (Fe NPs) to 110 nm (Fe-BB NPs). The hydrodynamic size and the polydispersity index (PDI) of nanoparticles showed negligible changes within 7 days (data not shown). Moreover, the zeta potential charge of nanoparticles decreased from +40 mV (Fc NPs) to −10 mV (Fe-BB NPs), and eventually stabilized, confirming the successful coating with BSA (FIG. 12D). These results demonstrate successful preparation of Fe NPs and Fe-BB NPs.

Further, the stability of Fe-BB NPs was examined and compared to Fe NPs. In vitro, the Fe NPs can precipitate in PBS owing to the positive charge on their surface, which leads to interaction with anions of PBS (FIGS. 12F and 12G). In contrast, the coated Fe NPs are more stable in PBS (FIGS. 12H and 12I).

### Photothermal Effect In Vitro and In Vivo

The ultraviolet-visible-NIR (UV-Vis-NIR) absorption spectra of Fe(II)-BQ-Bphen2 complex @BSA nanomedicine is shown in FIG. 12B, which showed the absorption in the NIR wavelength range. Under the 808 nm laser irradiation with different power densities, photothermal temperature increase of Fe(II)-BQ-Bphen2 complex @BSA at 100 μg/mL was measured using a thermal imaging camera. As shown in FIG. 13A, the temperature of the Fe-BB NPs solution increased with the increase of laser power density, with the maximum temperature elevation of 51.8° C. at 1.0 W/cm. In contrast, deionized water only had a weak temperature elevation (2.5° C.) even at the highest power density (1.0 W/cm²). Even at a moderate power density of 0.5 W/cm², the temperature of Fc-BB NPs solution still increased ˜26° C. The photothermal conversion efficiency (n) of Fe-BB NPs nanoparticles at 808 nm was calculated to be 60.6% based on the maximum steady-state temperature. This n value of Fe NPs is higher than currently reported photothermal agents such as Au nanorods (21%), Cu₂-xSe (22%), and Cu₉S₅ (25.7%), demonstrating that Fe NPs possesses a high capability to convert NIR irradiation to heat.

The temperature of Fe-BB NPs solutions increased with the increase of solution concentration, where the temperature increased 18.5° C. even at a low concentration (12.5 μg/mL) (FIG. 13B). These results demonstrate the excellent and dose-dependent photothermal effect of the Fe-BB NPs. The photothermal stability of the Fe-BB NPs was also evaluated (FIG. 13C). As the laser was switched on and off repeatedly for four times, the maximum temperature rise did not change significantly, demonstrating the good photothermal stability of the Fe-BB nanoparticles (FIG. 13D).

The photothermal curve of tumors on mice during 808 nm laser irradiation 6 min after 1 h via intravenous tail vein injection of the Fe(II)-BQ-Bphen2 complex nanoparticles (10 mg/kg) is shown in FIG. 13E. As shown in FIG. 13E, the PBS control group showed about 3.7° C. of temperature increase at the tumor site under 0.5 W/cm² 808 nm laser irradiation for 6 min, while the Fe NPs@BSA treatment group exhibited about 13.8° C. of temperature increase, demonstrating that the coated Fe NPs effectively accumulated in the tumor site via a passive targeting way (i.e., EPR effect) and had significant photothermal effect for thermal therapy of cancer.

Further, these in vivo experiments showed that mice treated with Fe NPs without coating exhibited weak photothermal effect under an 808 nm laser irradiation (0.5 W/cm², 6 min), indicating that there were few Fe nanoparticles accumulated in the tumor sites (FIGS. 13E and 13F). In contrast, Fe NPs coated with BSA exhibited strong photothermal effect in tumor sites, demonstrating that an appreciable amount of coated Fe NPs accumulated in the tumor sites (FIGS. 13E and 13F).

These results demonstrate the excellent photothermal performance of Fe-BB NPs in vitro and in vivo.

### Photothermal Efficacies In Vitro

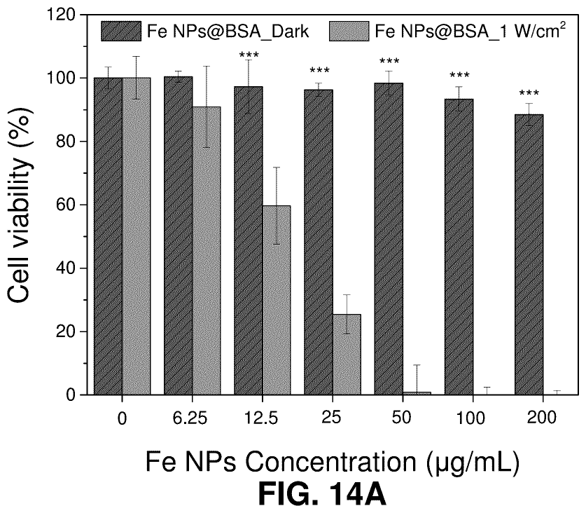

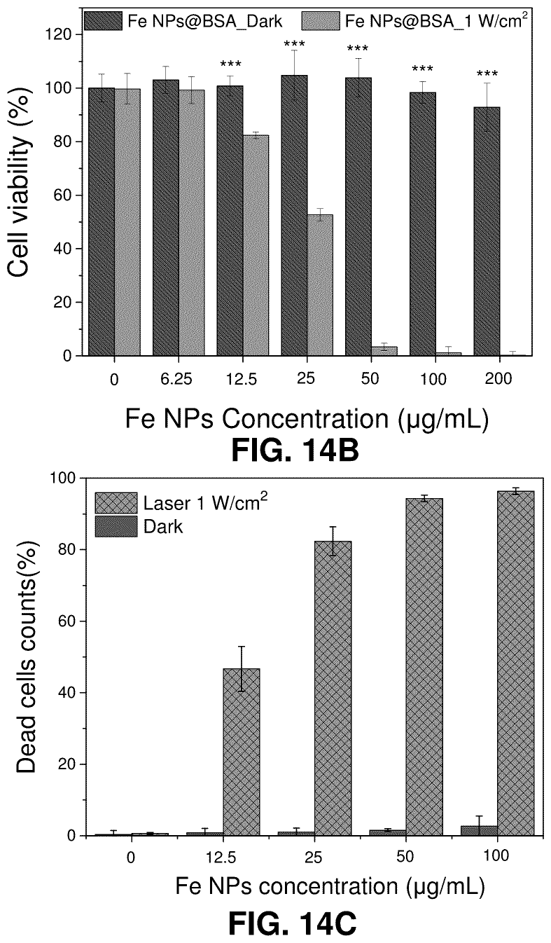

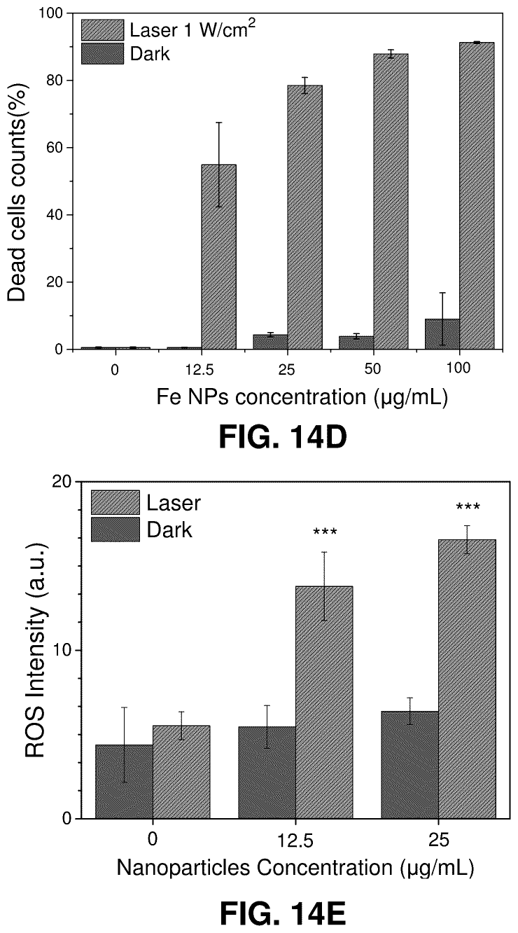

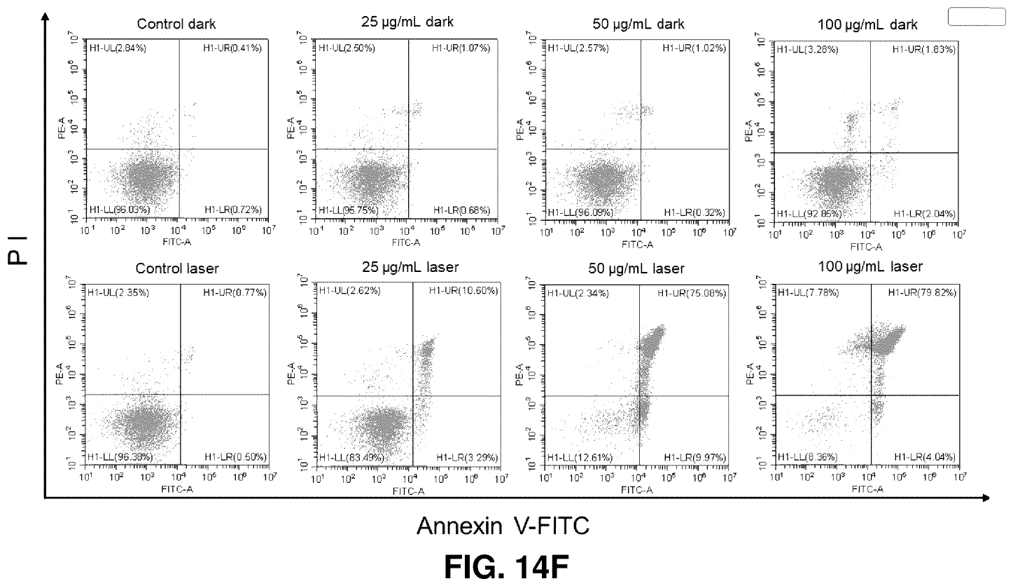

The Fe-BB NPs was further studied for cancer treatment. The cytotoxicity of Fe-BB NPs against cancer cells (NCI H460 and 4T1 cells) under 808-nm laser irradiation was investigated using MTT assay (FIGS. 14A-14B). In the absence of 808-nm laser irradiation, the Fe-BB NPs did not cause a significant decrease in cell viability. Even at a high concentration of 200 μg/mL, the maximum viability decrease of 4T1 and NCI H460 cells did not exceed 10%, showing the low cytotoxic of the Fe-BB NPs. The Fe-BB NPs showed concentration- and laser power density-dependent cytotoxicity against cancer cells under 808 nm laser irradiation. As shown in FIGS. 14C-14D, the microscopy count of dead cells and whole cells using propidium iodide and Hoechst stain assay support the dosage-dependent cytotoxicity of the Fe-BB NPs against cancer cells with NIR and biosafety of the Fe-BB NPs in dark. To study the localization of Fe-BB NPs in cells, the Fc(II)-BQ-Bphen2 complex was co-assembled with Dir fluorescent dye to form Fe nanoparticles. After coated with BSA, the Fe-BB nanoparticles were incubated with NCI H460 cells. Confocal laser scanning microscopy (CLSM) images of NCI H460 cells after co-incubation with Dir-labeled Fe NPs showed that the Fe-BB nanoparticles entered the lysosome within 2 h incubation (data not shown).

To directly observe the photothermal treatment effect of the Fe-BB NPs, NCI H460 cells treated with the Fe-BB NPs were co-stained with calcein acetoxymethyl ester/propidium iodide (Calcein-AM/PI) to differentiate live and dead cells (data not shown). For cells incubated with culture medium only, there was no clear cell death in the illuminated area, similar to the dark area, indicating that simple laser irradiation had no special killing effect on cancer cells. After treated with Fe-BB NPs, laser irradiation on cells resulted in a significant increase in the number of dead cells, due to the killing effect of hyperthermia on cancer cells.

Hypothermia can cause mitochondria damage and induce ROS in cells. To measure the ROS level in cells, DCFH-DA probe was used to verify the cellular ROS production. As shown in FIG. 14E, cells with vehicle treatment produced the least ROS in the dark and under laser irradiation. When treated with Fe-BB NPs, the cells produced significantly more ROS under laser irradiation. Further, the cellular ROS increased with the increase of Fe-BB nanoparticle's concentration, under laser irradiation. To observe cellular DNA damage level, γH2AX was stained using immunofluorescence in NCI-H460 cells. As shown in CLSM images of NCI H460 cells using γ-H2AX and DAPI saining assay (data not shown), cells treated with PBS showed negligible DNA damage with or without laser irradiation. Cells treated with Fe-BB nanoparticles in the dark caused observable amount of DNA damage. However, the level of DNA damage was most significant when treated with Fe-BB nanoparticles under laser irradiation, which support the cells cytotoxicity results described above.

The cell death mechanism was studied using flow cytometry based on Annexin V-FITC/propidium iodide (PI) assay. As shown in FIG. 14F, Fe-BB nanoparticles caused negligible cell apoptosis and death in the dark, which was similar to PBS treated cells. However, upon laser irradiation, the Fe-BB nanoparticles induced a significantly increased incidence of early-stage and late-stage apoptosis in 4T1 cells. When the Fe-BB nanoparticles concentration increased, the apoptotic rates increased significantly to 75.08% (50 μg/mL) and 79.82% (100 μg/mL) from 10.6% (50 μg/mL), respectively, confirming the role of ROS in the photocytotoxicity of the Fe-BB nanoparticles.

### In Vivo Photothermal Therapy

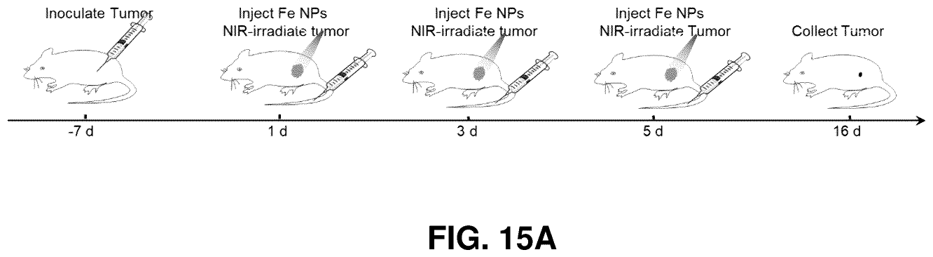

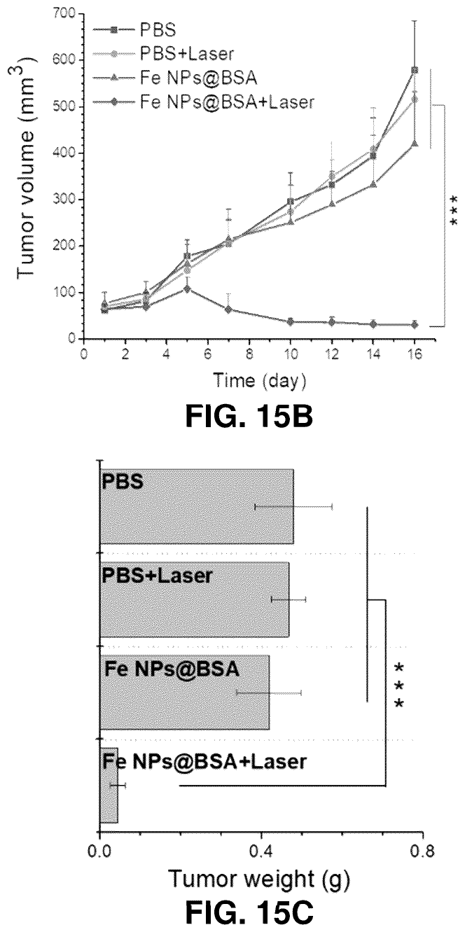

The photothermal effect of Fe-BB NPs were then studied for tumor therapy of mice in vivo according to the procedure described in the method section above. Mice treated by intravenous injection of Fe-BB NPs were executed (FIG. 15A). Mice bearing 4T1 tumor were randomly divided into four groups (PBS, Fe-BB NPs with or without laser irradiation) to investigate the anticancer effect of Fe-BB NPs using photothermal treatment. After PTT treatment, tumor growth of mice was not affected in the vehicle group, while Fe-BB NPs treated group under laser irradiation inhibited tumor growth significantly (FIGS. 15B-15C). These results are in accordance with the in vitro results (FIGS. 14A-14F). By measuring the weights and sizes of extracted tumors among all groups 16 days after treatment, the therapeutic effects of Fe-BB NPs were further confirmed, where the PTT group showed the best treatment results (FIGS. 15B-15C). At the end of the treatment, the tumors of mice were dissected from all groups followed by hematoxylin and cosin staining (H&E) (data not shown). In the vehicle group, lots of stromal tight cells existed, and the nucleus and cytoplasm were intact, showing that the tumor cells were in a good condition.

In order to eradicate tumors, photothermal needs to be heated to a high temperature, but high temperature could also damage the normal tissues around the tumors. If a mild temperature is used, it is difficult to kill the tumors completely. One solution is to activate the immune response through mild PTT or combined PTT.

4T1 tumors-bearing mice that have low immune response with insufficient immunogenicity were used to further validate that mild PTT could convert tumors from “cold” to “hot.” The 4T1-tumor-bearing BALB/c mice were randomly grouped, and each treated with a single intratumor injection of PBS or Fe nanoparticles when the tumor volumes reached about 50 mm³. The dose of Fe nanoparticles was 10 mg/kg. NIR irradiation with 808 nm laser (0.3 W/cm², 6 min) was applied to the mice treated with PBS or Fe nanoparticles on days 1 and 3 post injection to achieve a mild PTT.

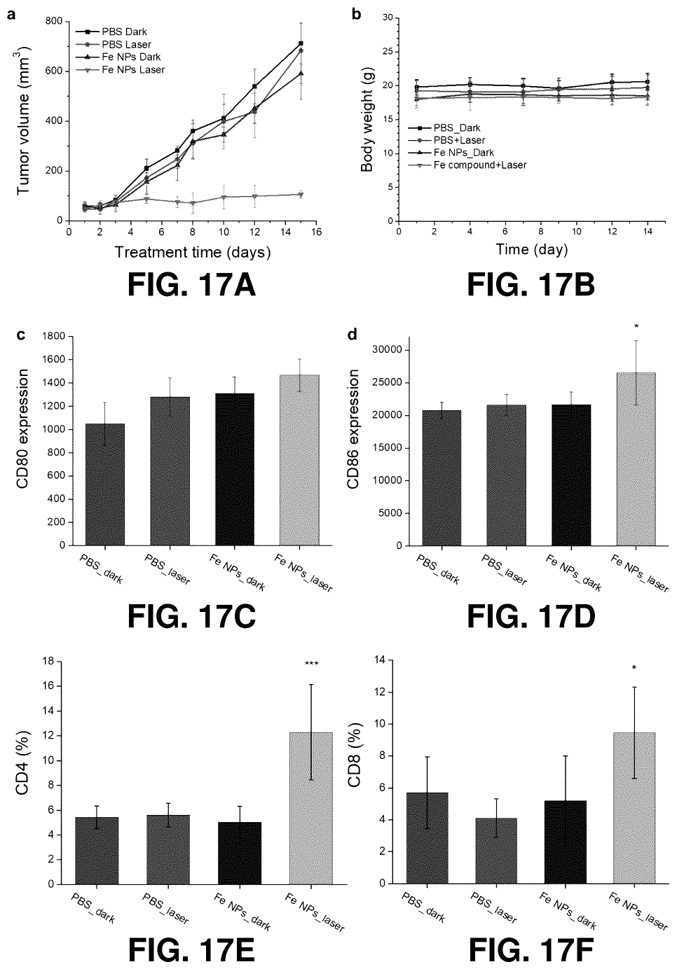

Further, it was found that the Fe nanoparticles can activate the immune response in vivo while the mild PTT was performed, thus achieving antitumor immunity effect. The mild PTT in combination with the activated antitumor immunity showed an effective inhibition on the growth of tumors (FIG. 17A), without observable weight changes of mice (FIG. 17B). To verify the enhanced antitumor immune response induced by the combined mild PTT and activated antitumor immunity, the response of immune cells in tumors and lymph nodes were measured (n=6) after the mice had been treated for 15 days. It is known that dendritic cells (DCs) play a role in initiating and regulating innate and adaptive immunities. Therefore, whether the mild PTT could activate DC maturation in the tumors was investigated. The percentage of matured DCs (CD80+CD86+) in the mild PTT group was significantly higher than that in the PBS group (FIGS. 17C and 17D).

These results demonstrated that mild PTT could induce immune stimulation effect. Meanwhile, the individual population of CD8+ T cells and CD4+ helper T cells in the primary tumors was analyzed. CD8+ T cells and CD4+ T cells are both T lymphocytes and are important in the immune responses of antitumor therapy. The populations of CD8+ T cells and CD4+ T cells in the mild PTT group were higher than those in the PBS group (FIGS. 17E and 17F), demonstrating the effective infiltration of cytotoxic T cells into 4T1 tumors.

### In Vivo Toxicity

The toxicology of Fe-BB nanoparticles in vivo was investigated systematically. Female Balb/c mice (4-6 weeks old) were randomly divided into 4 groups and treated with different conditions: (1) control group without laser irradiation, (2) control group with laser irradiation for 6 min (3) Fe-BB nanoparticles treatment via directly intravenously injection without laser irradiation and (4) Fe-BB nanoparticles treatment via directly intravenously injection after 808 nm laser irradiation for 6 min. The injection dose of the Fe-BB nanoparticles was 10 mg/kg. Haematological, blood biochemical and histological analyses were performed 16 days post-injection.

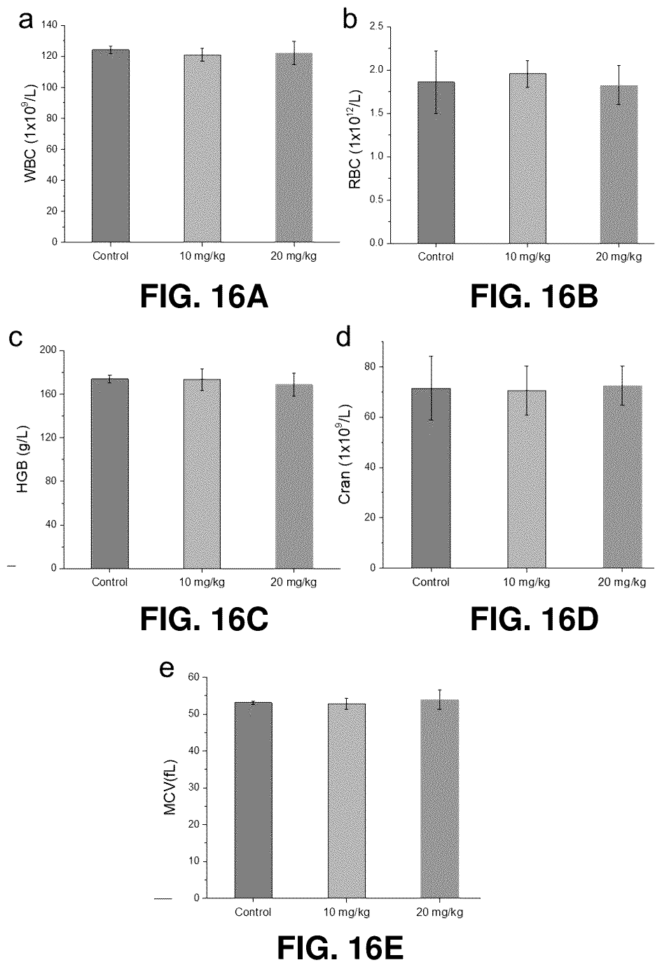

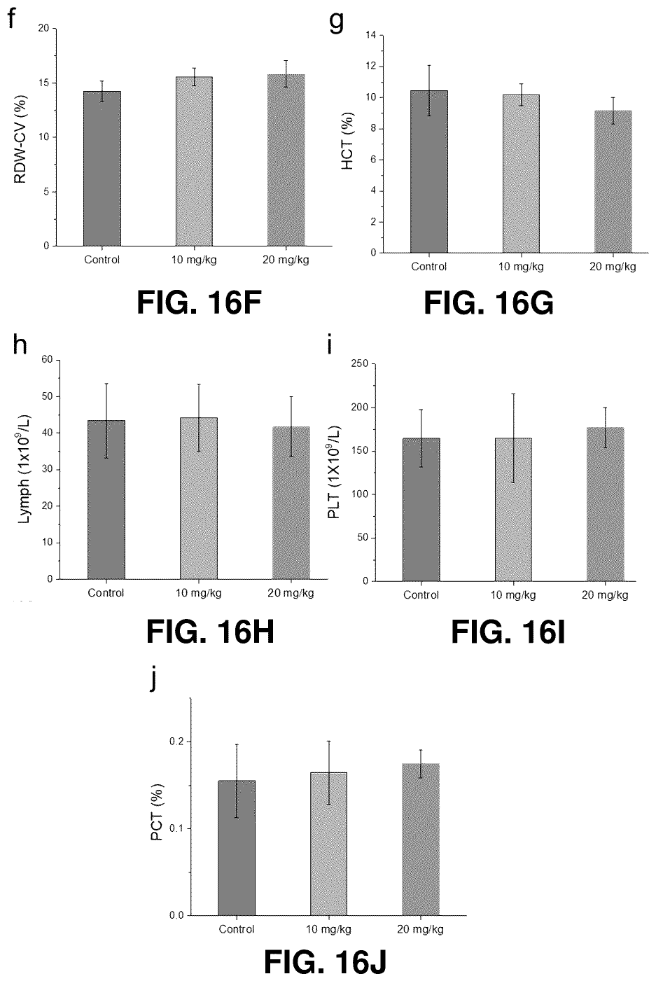

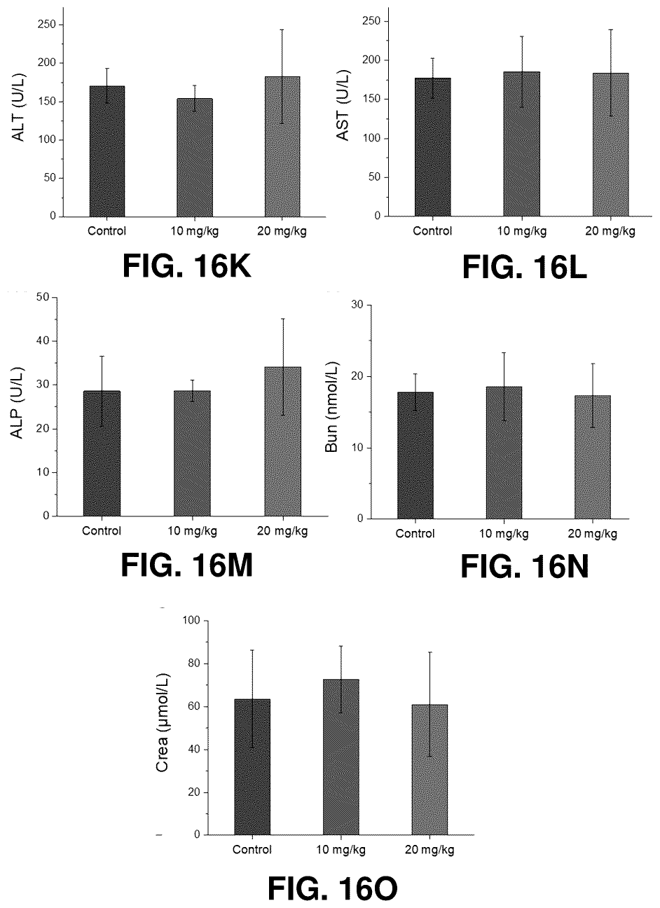

The standard hematology markers including white blood cells, red blood cells, haemoglobin, mean corpuscular volume, mean corpuscular haemoglobin, mean corpuscular haemoglobin concentration, platelets and haematocrit were measured (FIGS. 16A-16J). The differences between the control group and Fe-BB nanoparticle-treated groups (10 mg/kg or 20 mg/kg) were statistically insignificant. Thus, the Fe-BB nanoparticle-treated groups appear to be normal. These results show that the Fe-BB nanoparticles did not cause noticeable infection and inflammation in the treated mice.

Blood biochemical analyses were performed and various parameters including alanine transaminase (ALT), aspartate transaminase (AST), alkaline phosphatase (ALP), blood urea nitrogen (BUN) and creatinine (CREA) were examined (FIGS. 16K-16O). Compared with the control group, no statistically meaningful difference was observed in the Fe-BB nanoparticle's treatment groups at the end time point. These results show that the Fe-BB nanoparticles treatment did not affect the blood chemistry of mice. Since alanine transaminase, aspartate transaminase, and creatinine are closely related to the functions of the liver and kidney of mice, the results demonstrate that the Fe-BB nanoparticles did not induce noticeable hepatic and kidney toxicity in mice.

The corresponding histological changes of organs were checked by immunohistochemistry using major organs including the liver, spleen, kidney, heart and lung collected and sliced for haematoxylin and cosin staining (data not shown). No noticeable organ damage was observed during the entire treatment period in all groups, indicating no apparent histological difference.

### REFERENCES

O. S. Wenger, J. Am. Chem. Soc. 2018, 140, 13522-13533;
J. K. McCusker, Science 2019, 363, 484.
C. Förster and K. Heinze, Chemi. Soc. Rev. 2020, 49, 1057-1070.
O. S. Wenger, Chem. Eur. J. 2019, 25, 6043-6052;
J. Chen and W. R. Browne, Coord. Chem. Rev. 2018, 374, 15-35.
J. Wu, et al., Inorganics 2020, 8.
J. D. Braun, et al., Nature Chem. 2019, 17, 1144-1150.
T. Jiang, et al., Proc. Natl. Acad. Sci. U.S.A 2020, 117, 20430.
P. Dierks, et al., Inorg. Chem. 2020.
B. C. Paulus, et al., Nature 2020, 582, 214-218.
Y. Liu, et al., Acc. Chem. Res. 2016, 49, 1477-1485.
L. Lindh, et al., Catalysts 2020, 10.
P. Chabera, et al., J. Phys. Chem. Lett. 2018, 9, 459-463.
M. Albrecht, Chem. Rev. 2010, 110, 576-623.
Z. Tang, et al., Organometallics 2020, 39, 2791-2802.
J. Steube, et al., Chem. Eur. J. 2019, 25, 11826-11830.
P. Aguirre-Etcheverry and D. O'Hare, Chem. Rev. 2010, 110, 4839-4864.
S. Campagna, et al., in Photochemistry and Photophysics of Coordination Compounds I (Eds.: V. Balzani, S. Campagna), Springer Berlin Heidelberg, Berlin, Heidelberg, 2007, pp. 117-214.
Y.-W. Zhong, et al., Coord. Chem. Rev. 2016, 312, 22-40.
Y. Shi, et al., Organometallics 2009, 28, 2206-2210.
L.-Z. Sui, et al., Inorg. Chem. 2012, 51, 1590-1598.
P. G. Bomben, et al., Inorg. Chem. 2009, 48, 9631-9643.
E. C. Constable and J. M. Holmes, J. Organomet. Chem. 1986, 301, 203-208.
Z. Zuo, et al., Dalton Trans. 2014, 43, 11716-11722.
L. Wang, ct al., Eur. J. Inorg. Chem. 2016, 2016, 5205-5214.
Y. Liu, et al., Chem. Eur. J. 2015, 21, 3628-3639.
I. M. Dixon, et al., Dalton Trans. 2015, 44, 13498-13503.
A. M. Brown, et al., Dalton Trans. 2014, 43, 17635-17646.
C. Consani, et al., Angew. Chem. Inter. Ed. 2009, 48, 7184-7187.
J. E. Monat and J. K. McCusker, J. Am. Chem. Soc. 2000, 122, 4092-4097.
P. S. Braterman, et al., Inorg. Chem. 1992, 31, 555-559.
T. Bessho, et al., J. Am. Chem. Soc. 2009, 131, 5930-5934.
C. Kreitner and K. Heinze, Dalton Trans. 2016, 45, 13631-13647.
M. L. Muro-Small, et al., Eur. J. Inorg. Chem. 2012, 2012, 4004-4011.
Z. Ji, et al., J. Phys. Chem. C 2012, 116, 16854-16863.
Q. Sun, et al., Coord. Chem. Rev. 2015, 282-283, 87-99.
A. D. Ryabov, et al., Inorg. Chem. 2001, 40, 6529-6532.
S. Finck, et al., J. Organomet. Chem. 2014, 760, 248-259.
P. Reveco, et al., Inorg. Chem. 1986, 25, 1842-1845.
J. A. Solís-Ruiz, et al., Journal of Inorganic Biochemistry 2020, 208, 111080.
T. Hofbeck and H. Yersin, Inorg. Chem. 2010, 49, 9290-9299;
S. Tschierlei, et al., Phys. Chem. Chem. Phys. 2016, 18, 10682-10687.
R. Bevernacgic, et al., Phys. Chem. Chem. Phys. 2018, 20, 27256-27260.
A. Cannizzo, et al., Coord. Chem. Rev. 2010, 254, 2677-2686.
Li, X., et al., Nat Rev Clin Oncol 2020, 17 (11), 657-674.
Yao, Y., et al., J Am Chem Soc 2022, 144 (16), 7346-7356.
Shao, J., et al., Nat Commun 2016, 7, 12967.
Liu, Y., et al., Nat Commun 2020, 11 (1), 1724.
Hu, K., et al., Nat Commun 2020, 11 (1), 2778.
Ali, M. R. K., et al., The Journal of Physical Chemistry C 2019, 123 (25), 15375-15393.
Liu, Y., et al., Chem Soc Rev 2019, 48 (7), 2053-2108.
<Electronic structure in the transition metal block and its implications for light harvesting.pdf>
Wenger, O. S., Chemistry 2019, 25 (24), 6043-6052.
Ng, K. K., et al., Chem Rev 2015, 115 (19), 11012-42.
Wang, Y., et al., ACS Applied Polymer Materials 2020, 2 (10), 4258-4272.
Tang, Z., et al., Organometallics 2020, 39 (15), 2791-2802.
Steube, J., et al., Chemistry 2019, 25 (51), 11826-11830.
Dixon, I. M., et al., Dalton Trans 2015, 44 (30), 13498-503.
Aggarwal, P., et al., Adv Drug Deliv Rev 2009, 61 (6), 428-37.
Fleischer, C. C., et al., Acc Chem Res 2014, 47 (8), 2651-9.
Xia, B., ct al., ACS Appl Mater Interfaces 2013, 5 (22), 11718-24.
<Hydrophilic Cu9S5 nanocrystals: a photothermal agent with a 25.7% heat conversion efficiency for photothermal ablation of cancer cells in vivo.pdf>.
Hessel, C. M., et al., Nano Lett 2011, 11 (6), 2560-6.
Lci, P., et al., Advanced Functional Materials 2017, 27 (35).
Aggarwal, P., ct al., Advanced Drug Delivery Reviews 2009, 61 (6), 428-437.
Fleischer, C. and Payne, C., Accounts of Chemical Research 2014, 47 (8), 2651-2659.
Elsadek, B. and Kratz, F., Journal of Controlled Release 2012, 157 (1): 4-28.
Blanco, E., et al., Nature Biotechnology 2015, 33 (9), 941-951.
Xu, G., et al., Nature Communications, 2022, 13 (1), 3064.
Karges, J., et al., European Journal of Inorganic Chemistry, 2020, 34, 3242-3248.
Yu, W., et al., Advanced Functional Materials, 2020, 30 (49), 2002239.
Meng, J., et al., Science Advances, 2020, 6 (20), eaba1362.
Xi, D., et al., Advanced Materials, 2022, 34 (6), 2106797.
Yang, Zhe, et al., Nature Reviews Clinical Oncology 20.2 (2023): 116-134.
Nam, Jutaek, et al., Nature Reviews Materials 4.6 (2019): 398-414.
Huang, Liping, et al., Nature communications 10.1 (2019): 4871.
Sagiv-Barfi, Idit, et al., PNAS 112.9 (2015): E966-E972.
Higgins, Jack P., et al., Cancer biology & therapy 8.15 (2009): 1440-1449.
Wen, Yi, and Joel H. Collier. Current opinion in immunology 35 (2015): 73-79.
Mahjub, Reza, et al., Journal of Controlled Release 288 (2018): 239-263.
Krieg, Arthur M. Nature reviews Drug Discovery 5.6 (2006): 471-484.
Guo, Liangran, et al. ACS nano 8.6 (2014): 5670-5681.
Kasturi, S. P. et al. Nature 470, 543-550 (2011).
Le Mercier, Isabelle, et al. Cancer Research 73.15 (2013): 4629-4640.
Chen, Qian, et al. Nature Communications 7.1 (2016): 13193.
Huang, Liping, et al. Nature Communications 10.1 (2019): 4871.
Guo, L. et al. ACS Nano 8, 5670-5681 (2014).
Wang, Chao, et al. Advanced Materials 48 (2014): 8154-8162.
Liu, Yang, et al. Scientific Reports 7.1 (2017): 8606.
Bear, A. S. et al. PLOS ONE 8, e69073 (2013).
Nam, Jutaek, et al. Nature Reviews Materials 4.6 (2019): 398-414.
Chen, W. R., et al. Cancer Res. 61, 459-461 (2001).
Toraya-Brown, S. et al. Nanomedicine 10, 1273-1285 (2014).
Shi, H. et al. J. Immunol. 176, 2134-2141 (2006).

We claim:

1. An iron (II) complex having a structure of: 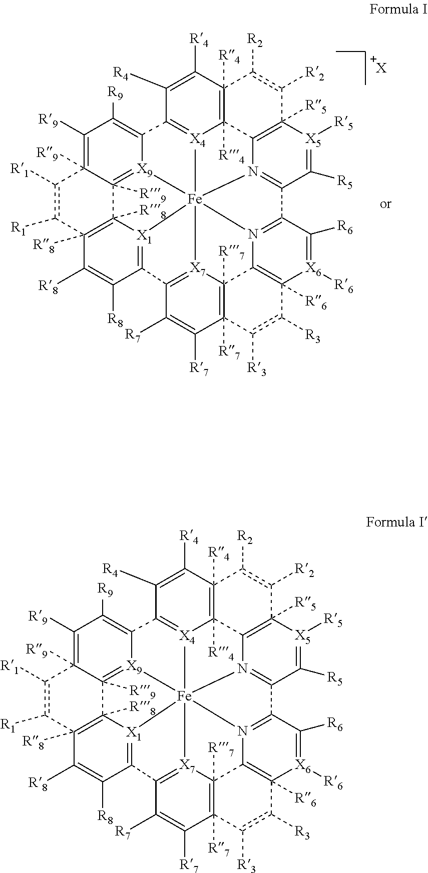
wherein:
(i) X₁, X₄, X₅, X₆, X₇, and X₉ are independently carbon or nitrogen, X is an anion;
(ii) each [symbol] is absent or a single bond, each [symbol] is absent or a double bond;
(iii) R₁, R′₁, R₂, R′₂, R₃, and R′₃, when present, are independently hydrogen, substituted or unsubstituted alkyl, substituted or unsubstituted aryl, or substituted or unsubstituted alkylaryl;
(iv) R₄, R′₄, R₅, R′₅, R₆, R′₆, R₇, R′₇, R₈, R′₈, R₉, and R′₉ are independently hydrogen, substituted or unsubstituted alkyl, substituted or unsubstituted aryl, substituted or unsubstituted heteroaryl, substituted or unsubstituted polyaryl, substituted or unsubstituted heteropolyaryl, substituted or unsubstituted alkylaryl, substituted or unsubstituted cyclic, substituted or unsubstituted heterocyclic, halide, amino, amido, thiol, hydroxyl, cyano, nitro, carbonyl, or alkoxyl;
(v) R″₄, R′″₄, R″₅, R″₆, R″₇, R′″₇, R″₈, R′″₈, R″₉, and R′″₉, when present, are independently hydrogen, halide, substituted or unsubstituted alkyl, substituted or unsubstituted aryl, substituted or unsubstituted heteroaryl, substituted or unsubstituted cyclic, or substituted or unsubstituted heterocyclic; and
(vi) the substituents, when present, are independently unsubstituted alkyl, unsubstituted aryl, unsubstituted heteroaryl, unsubstituted polyaryl, unsubstituted heteropolyaryl, unsubstituted alkylaryl, unsubstituted cyclic, unsubstituted heterocyclic, halide, amino, amido, thiol, hydroxyl, cyano, nitro, carbonyl, or alkoxyl.

2. The iron (II) complex of claim 1, having a structure of: 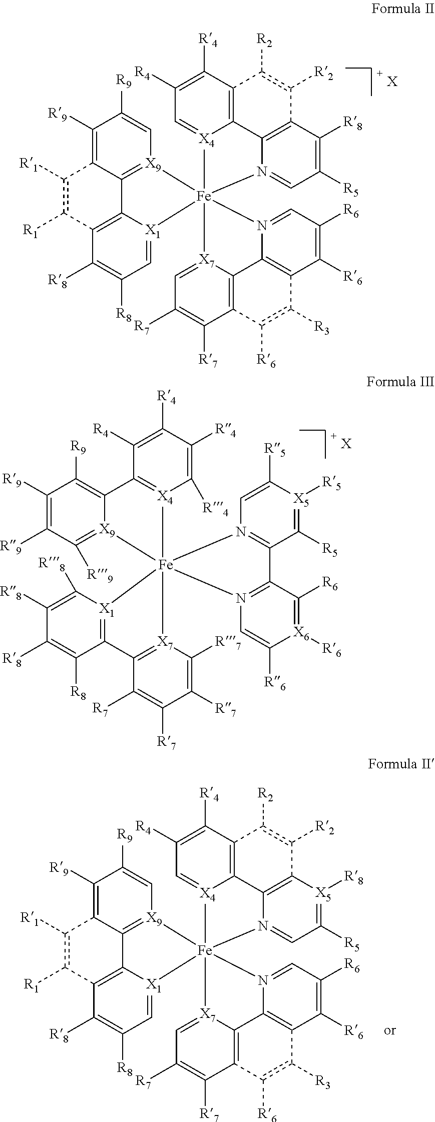 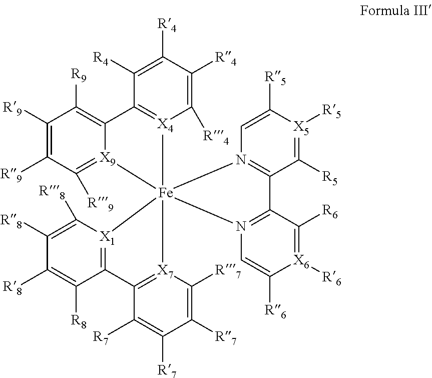
wherein:
(i) X₁, X₄, X₅, X₆, X₇, and X₉ are independently carbon or nitrogen, X is an anion;
(ii) each [symbol] is absent or a single bond, each [symbol] is absent or a double bond;
(iii) R₁, R′₁, R₂, R′₂, R₃, and R′₃, when present, are independently hydrogen, substituted or unsubstituted alkyl, or substituted or unsubstituted aryl;
(iv) R₄, R′₄, R″₄, R′″₄, R₅, R′₅, R″₅, R₆, R′₆, R″₆, R₇, R′₇, R″₇, R′″₇, R₈, R′₈, R′″₈, R′″₈, R₉, R′₉, R″₉, and R′″₉ are independently hydrogen, substituted or unsubstituted alkyl, substituted or unsubstituted aryl, substituted or unsubstituted heteroaryl, substituted or unsubstituted heterocyclic, or halide; and
(v) the substituents, when present, are independently unsubstituted alkyl, unsubstituted aryl, unsubstituted heteroaryl, unsubstituted polyaryl, unsubstituted heteropolyaryl, unsubstituted alkylaryl, unsubstituted cyclic, unsubstituted heterocyclic, halide, amino, amido, thiol, hydroxyl, cyano, nitro, carbonyl, or alkoxyl.

3. The iron (II) complex of claim 1, wherein:
(i) X₁, X₅, and X₆ are carbon, and X₄, X₇, and X₉ are nitrogen; or
(ii) X₁, X₉, X₅, and X₆ are carbon, and X₄ and X₇ are nitrogen; or
(iii) X₄, X₇, and X₈ are carbon, and X₁, X₉, and X₆ are nitrogen.

4. The iron (II) complex of claim 1, wherein:
(i) R₁, R′₁, R₂, R′₂, R₃, and R′₃, when present, are independently hydrogen, unsubstituted alkyl, or unsubstituted aryl;
(ii) R₄, R′₄, R₅, R′₅, R₆, R′₆, R₇, R′₇, R₈, R'₈, R₉, and R′₉, are independently hydrogen, unsubstituted alkyl, unsubstituted aryl, unsubstituted heteroaryl, or halide; and
(iii) R″₄, R′″₄, R″₅, R″₆, R″₇, R′″₇, R″₈, R′″₈, R″₉, and R′″₉, when present, are independently hydrogen, unsubstituted heteroaryl, or halide.

5. The iron (II) complex of claim 1, having a structure of:

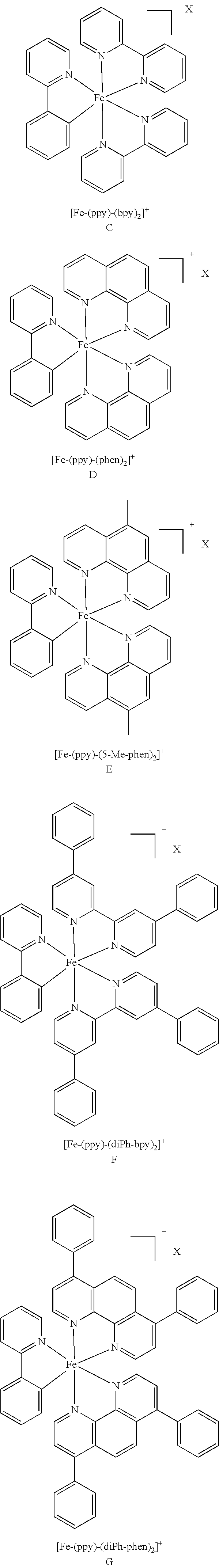

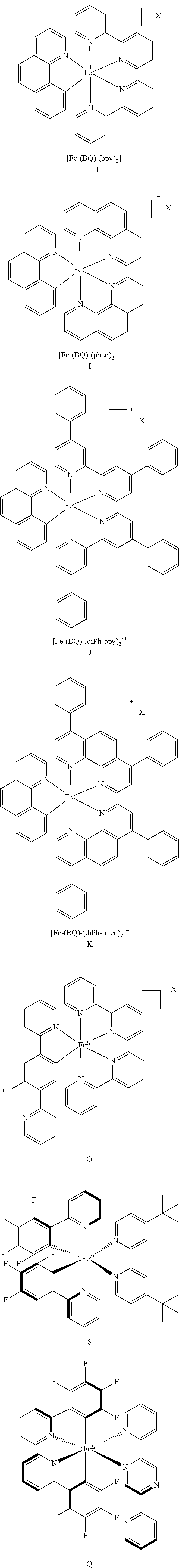

6. The iron (II) complex of claim 1, having an absorption at a wavelength of at least 600 nm, such as ranging from 600 nm to about 850 nm or from about 700 nm to about 850 nm, and/or a lifetime of at least 1 ps.

7. Metallosupramolecular particles comprising the iron (II) complex of claim 1.

8. The metallosupramolecular particles of claim 7, further comprising a coating agent, wherein the coating agent is covalently or non-covalently attached to the surface of the metallosupramolecular particles, and optionally wherein the coating agent is bovine serum albumin, polyalkylene or a copolymer thereof (e.g., Pluronic® F-127, DSPE-PEG (2000)), or a polysaccaride (e.g., hyaluronic acid), or a combination thereof.

9. The metallosupramolecular particles of claim 7, further comprising a targeting moiety.

10. The metallosupramolecular particles of claim 7, having an average morphology diameter ranging from about 50 nm to about 150 nm or from about 80 nm to about 100 nm, as measured using transmission electron microscopy; and/or an average hydrodynamic diameter ranging from about 60 nm to about 200 nm, such as about 90 nm, as measured using dynamic light scattering.

11. The metallosupramolecular particles of claim 7, having a photo-heat conversion efficiency of at least 30%, at least 40%, at least 50%, or at least 60%, measured using a 808 nm laser irradiation at 1.0 W/cm for about 20 min; and/or a photothermal stability for at least four laser irradiation-cooling cycles, each laser irradiation lasts at least 5 mins.

12. A pharmaceutical composition comprising the metallosupramolecular particles of claim 7, and optionally one or more pharmaceutically acceptable excipients.

13. The pharmaceutical composition of claim 12, wherein the metallosupramolecular particles are in an amount ranging from about 1 μM to about 1 mM, from about 10 μM to about 500 μM, from about 10 μM to about 200 μM, or from about 25 μM to about 100 μM.

14. A method for treating cancer using the pharmaceutical composition of claim 12, comprising:
(i) administering the pharmaceutical composition to a subject in need thereof; and
(ii) applying a laser irradiation to the subject or a target region of the subject, wherein step (i), step (ii), or steps (i) and (ii) occurs one or more times.

15. The method of claim 14, wherein in step (ii) the laser irradiation:
(a) has a wavelength of at least 670 nm, in a range from 670 nm to 1500 nm, from 670 nm to 1200 nm, from 670 nm to 1000 nm, from 700 nm to 1500 nm, from 700 nm to 1200 nm, or from 700 nm to 1000 nm, such as about 800 nm;
(b) has a laser power density ranging from about 0.1 W/cm to about 10 W/cm, from about 0.1 W/cm to about 5 W/cm, from about 0.1 W/cm to about 1 W/cm, from about 0.1 W/cm to about 0.5 W/cm, or from about 0.1 W/cm to about 0.3 W/cm, such as about 1 W/cm, about 0.5 W/cm, or about 0.3 W/cm; and/or
(c) is maintained for a time period ranging from about 1 min to about 1 hour, from about 5 mins to about 1 hour, from about 1 min to about 30 mins, from about 5 mins to about 30 mins, from about 1 min to about 20 mins, from about 5 mins to about 20 mins, from about 1 min to about 15 mins, or from about 5 mins to about 15 mins.

16. The method of claim 14, wherein the pharmaceutical composition is administered by oral administration, intramuscular administration, intravenous administration, intraperitoneal administration, or subcutaneous administration, or a combination thereof.

17. The method of claim 14, further comprising administering an active agent, optionally wherein the active agent is an anticancer agent.

18. The method of claim 14, wherein in step (i), the dosage of the metallosupramolecular particles administered is from about 0.1 μg to about 100 μg, from about 0.5 μg to about 50 μg, from about 1 μg to about 100 μg, from about 1 μg to about 50 μg, from about 1 μg to about 25 μg, from about 1 μg to about 10 μg, from about 1 μg to about 5 μg, from about 4 μg to about 10 μg, or from about 1 μg to about 4 μg per g of the subject.

19. The method of claim 14, wherein following step (ii) or all of step (ii) (if step (ii) occurs more than one time), the tumor volume is reduced by at least 10%, at least 20%, at least 30%, at least 40%, or at least 50%, compared to the tumor volume before administration of the pharmaceutical composition; and/or the tumor weight is reduced by at least 50%, at least 60%, at least 70%, or at least 80% compared to the tumor weight before administration of the pharmaceutical composition, optionally without any cytotoxicity to normal cells as indicated by standard hematology markers and/or blood biochemical parameters compared to a control administered with the pharmaceutically acceptable excipient(s) only.

20. An iron (II) complex having a structure of: 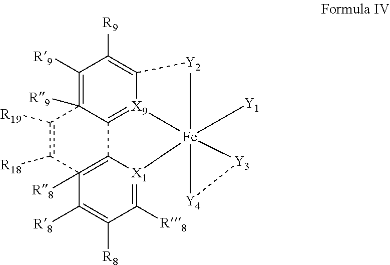
wherein (i) X₁ and X₉ are independently carbon or nitrogen; (ii) Y₁, Y₂, Y₃, and Y₄ are independently phosphorus, halogen, substituted or unsubstituted aryl, or substituted or unsubstituted heteroaryl; (iii) R₈, R′₈, R″₈, R″″₈, R₉, R′₉, R″₉, R₁₈ (when present), and R₁₉ (when present) are independently hydrogen, substituted or unsubstituted alkyl, substituted or unsubstituted aryl, substituted or unsubstituted heteroaryl, substituted or unsubstituted heterocyclic, or halide; and (iv) the substituents, when present, are independently unsubstituted alkyl, unsubstituted aryl, unsubstituted heteroaryl, unsubstituted polyaryl, unsubstituted heteropolyaryl, unsubstituted alkylaryl, unsubstituted cyclic (e.g., cycloalkyl, cycloalkenyl, cycloalkynyl), unsubstituted heterocyclic, halide, amino, amido, thiol, hydroxyl, cyano, nitro, carbonyl, or alkoxyl.

21. The iron (II) complex of claim 20, having a structure of: 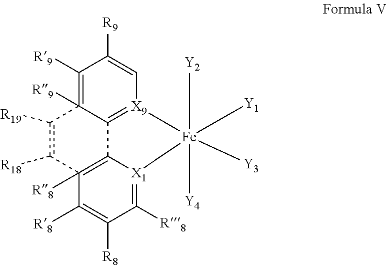
wherein (i) X₁ and X₉ are independently carbon or nitrogen; (ii) Y₁, Y₂, Y₃, and Y₄ are independently phosphorus or halogen; (iii) R₈, R′₈, R″₈, R′″₈, R₉, R′₉, R″₉, R₁₈ (when present), and R₁₉ (when present) are independently hydrogen, substituted or unsubstituted alkyl, or halide; and (iv) the substituents, when present, are independently unsubstituted alkyl, unsubstituted aryl, unsubstituted heteroaryl, unsubstituted polyaryl, unsubstituted heteropolyaryl, unsubstituted alkylaryl, unsubstituted cyclic (e.g., cycloalkyl, cycloalkenyl, cycloalkynyl), unsubstituted heterocyclic, halide, amino, amido, thiol, hydroxyl, cyano, nitro, carbonyl, or alkoxyl.

22. The iron (II) complex of claim 21, wherein:
X₁ is nitrogen and X₉ is carbon;
Y₁, Y₂, Y₃, and Y₄ are independently trialkylphosphine (e.g., trimethylphosphine) or halogen; or
R₈, R′₈, R″₈, R′″₈, R₉, R′₉, and R″₉ are independently hydrogen or unsubstituted alkyl; or
R″₈ and R″₉ are independently hydrogen; or
R₁₈ and R₁₉, when present, are independently hydrogen or unsubstituted alkyl; or
a combination thereof.

23. The iron (II) complex of claim 20, having a structure of: 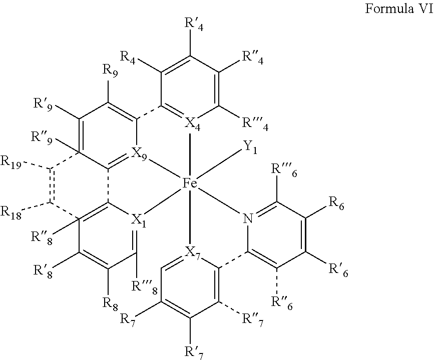
wherein (i) X₁, X₄, X₇, and X₉ are independently carbon or nitrogen; (ii) Y₁ is phosphorus, halogen, substituted or unsubstituted aryl, or substituted or unsubstituted heteroaryl; (iii) R₄, R′₄, R″₄, R′″₄, R₆, R′₆, R″₆, R′″₆, R₇, R′₇, R″₇, R₈, R′₈, R″₈, R′″₈, R₉, R′₉, R″₉, R₁₈ (when present), and R₁₉ (when present) are independently hydrogen, substituted or unsubstituted alkyl, or halide; and (iv) the substituents, when present, are independently unsubstituted alkyl, unsubstituted aryl, unsubstituted heteroaryl, unsubstituted polyaryl, unsubstituted heteropolyaryl, unsubstituted alkylaryl, unsubstituted cyclic (e.g., cycloalkyl, cycloalkenyl, cycloalkynyl), unsubstituted heterocyclic, halide, amino, amido, thiol, hydroxyl, cyano, nitro, carbonyl, or alkoxyl.

24. The iron (II) complex of claim 23, having a structure of: 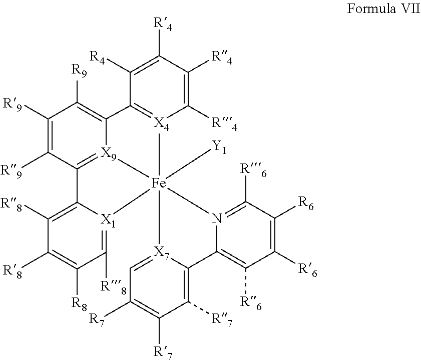
wherein (i) X₁, X₄, X₇, and X₉ are independently carbon or nitrogen; (ii) Y₁ is phosphorus or halogen; (iii) R₄, R′₄, R″₄, R′″₄, R₆, R′₆, R″₆, R′″₆, R₇, R′₇, R″₇, R₈, R′₈, R″₈, R′″₈, R₉, R′₉, and R″₉ are independently hydrogen, substituted or unsubstituted alkyl, or halide; and (iv) the substituents, when present, are independently unsubstituted alkyl, unsubstituted aryl, unsubstituted heteroaryl, unsubstituted polyaryl, unsubstituted heteropolyaryl, unsubstituted alkylaryl, unsubstituted cyclic (e.g., cycloalkyl, cycloalkenyl, cycloalkynyl), unsubstituted heterocyclic, halide, amino, amido, thiol, hydroxyl, cyano, nitro, carbonyl, or alkoxyl.

25. The iron (II) complex of claim 23, wherein:
X₁, X₄, and X₉ are nitrogen and X₇ is carbon;
Y₁ is halogen (e.g., fluorine, chlorine, or bromine); or
R₄, R′₄, R″₄, R′″₄, R₆, R′₆, R″₆, R′″₆, R₇, R′₇, R″₇, R₈, R′₈, R″₈, R′″₈, R₉, R′₉, and R″₉ are independently hydrogen or unsubstituted alkyl; or
R″₈ and R″₉ are independently hydrogen; or
R₁₈ and R₁₉, when present, are independently hydrogen or unsubstituted alkyl; or
a combination thereof.
